(19) United States
(12) Patent Application Publication
(10) Pub. No.: US 2026/0000784 A1
(43) Pub. Date: Jan. 1, 2026

(54) PEPTIDIC WATER-SOLUBLE DELIVERY SYSTEM OF ANTICANCER DRUGS

(71) Applicants: HOSPITAL SANT JOAN DE DEU, Esplugues de Llobregat (ES); TECHNION RESEARCH AND DEVELOPMENT FOUNDATION LIMITED, Haifa (IS); GATE2BRAIN, S.L., Barcelona (ES)

(72) Inventors: Ángel MONTERO CARCABOSO, Barcelona (ES); Alejandro DARIO SOSNIK, Haifa (IS); Clàudia RESA PARÉS, Manresa (ES); Meritxell TEIXIDÓ TURÁ, SANT JOAN DESPÍ (ES)

(73) Assignees: HOSPITAL SANT JOAN DE DEU, Esplugues de Llobregat (ES); TECHNION RESEARCH AND DEVELOPMENT FOUNDATION LIMITED, Haifa (IL); GATE2BRAIN, S.L., Barcelona (ES)

(21) Appl. No.: 18/852,242

(22) PCT Filed: Mar. 27, 2023
(86) PCT No.: PCT/EP2023/057869

§ 371 Date: Sep. 27, 2024

(30) Foreign Application Priority Data
Mar. 28, 2022 (EP) 22382287.5

(51) Int. Cl. A61K 47/69; A61K 9/107; A61P 35/00

(52) U.S. Cl. CPC A61K 47/6907; A61K 9/1075; A61P 35/00

(57) ABSTRACT

Micelles of peptidic conjugates of SN-38 loaded with one or more free therapeutic active agents which anticancer activity, such as free SN-38 lactone, process for their preparation, pharmaceutical compositions comprising them, and their therapeutical indications as anticancer drugs.

This application claims the benefit of European Patent Application EP22382287 filed 28 Mar. 2022.

### TECHNICAL FIELD

The present invention relates to the field of delivery systems comprising an anticancer drug, process for their preparation, and their therapeutical indications.

### BACKGROUND ART

Campthotecins are a family of topoisomerase I inhibitors with anticancer properties. They are chemically characterized by the presence of a lactone ring that confers the anticancer activity and is stable at acidic pH. The lactone ring opens to carboxylate at neutral or basic pH. Such conversion is non-enzymatic, and it is reversible to lactone upon pH acidification. The carboxylate is 100-1000 times less potent than the lactone.

Irinotecan is a camptothecin approved for the treatments of several types of cancer. SN-38 (7-ethyl-10-hydroxycamptothecin) is the active metabolite of irinotecan and is formed via hydrolysis of irinotecan, its water-soluble prodrug, by liver carboxylesterases and metabolized via glucuronidation by UGT1A1. It has the following formula.

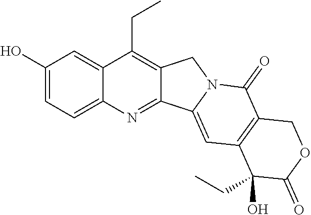 [SMILES: CCc1c2c(nc3ccc(O)cc13)-c1cc3c(c(=O)n1C2)COC(=O)[C@]3(O)CC]

SN-38 has 1000 times more activity than irinotecan itself and could be used to treat the same type of cancer than irinotecan. In vitro cytotoxicity assays show that the potency of SN-38 relative to irinotecan varies from 2- to 2000-fold.

SN-38 has significant limitations at the chemical, pharmacological and toxic level. At the chemical level, unlike irinotecan that is soluble in water at acidic pH, SN-38 is practically insoluble in water at acidic or neutral pH, and in most solvents and oils, so it is unfeasible to administer SN-38 to patients keeping the active (lactone) form of the molecule. Many solvents have been tested and it is only possible to solubilize SN-38 at 0.5%, with dimethyl sulfoxide, formic acid and Transcutol® HP, as well as with NaOH 0.1 M, but the basic pH of this NaOH 0.1 M water solution opens the lactone ring and thus inactivates SN-38. Thus, the high lipophilic character of SN-38 impedes the administration of this drug to human beings in clinically acceptable vehicles. At neutral or basic pH, the balance is shifted towards less active species, due to the opening of the lactone ring, while at more acidic pH the formation of lactone is favored, with greater cancer inhibitory power. The drug also contains an asymmetric carbon in position 20, with the S form being the pharmacologically active configuration.

In summary, two forms of SN-38 are in equilibrium in water, the lipophilic lactone (SN-38 lactone, with potent anticancer activity) and the hydrophilic carboxylate (SN-38 carboxylate, without anticancer activity). SN-38 lactone predominates under acidic pH conditions, and it is practically insoluble in water (<40 g/mL according to J. A: Zhang et al., Development and characterization of a novel liposome-based formulation of SN-38. International Journal of Pharmaceutics (2004), vol. 270(1-2), pp. 93-107), whereas SN-38 carboxylate predominates at neutral and basic pH, and it is freely soluble in water. Both SN-38 forms are in equilibrium and when SN-38 carboxylate solutions (basic pH) are poured into acid pH solutions, the carboxylate form is converted into SN-38 lactone, which precipitates and forms spike-shaped crystals, even in the presence of non-ionic surfactants such as Pluronic® F68 (see EP2644191B1). This precipitation process is associated with the strong inter-molecular interactions between free SN-38 lactone molecules.

As many other lipophilic small-molecule drugs, camptothecins self-assemble in water and form insoluble aggregates (see A. Sosnik, Drug self-assembly: A phenomenon at the nanometer scale with major impact in the structure-biological properties relationship and the treatment of disease, Prog Mater Sci 2016, vol. 82, pp. 39-82).

Given the unfavourable characteristics of the drug, several investigations have led to chemical conjugations of SN-38 with solubilizing chemical groups, to produce SN-38 prodrugs. Several conjugate strategies have been applied to SN-38 to release the drug instead of the use of the free drug SN-38.

Some of them are based on the release of SN-38 from a soluble conjugate. For instance, U.S. Pat. No. 8,299,089B2 proposes the use of multi-armed PEGs to conjugate the SN-38 through proper linkers to improve the solubility and release the SN-38 due to ester hydrolysis. However, these conjugates must be administered at high concentrations in order to be efficacious in vitro/in vivo.

Likewise, WO2015/051307A1 discloses several conjugates that release SN-38 from a 4-arm polyethylene glycol through a p-elimination reaction mechanism at slow rates to enable low-dose and long-term exposure regimes. Other conjugates are for instance based on the use of peptidic conjugates to by-pass the hepatic activation and reduce the gastrointestinal toxicity and interpatient variability compared to irinotecan. Thus, for instance, F Meyer-Losic et al in “DTS-108, A novel Peptidic prodrug of SN-38: In vivo Efficacy and Toxicokinetic Studies”, Clinical Cancer Research 2008, vol. 14, issue 7, pp. 2145-2153, proposes to conjugate SN-38 to a cationic peptide (Vectocell) via an esterase cleavable linker to deliver significantly higher levels of SN-38 than irinotecan, without the associated toxicity of irinotecan, resulting in an increased therapeutic window for DTS-108 in preclinical models.

Other strategies for the solubilization of free SN-38 lactone in aqueous solutions have also been disclosed in the state of the art which encompass the use of amphiphilic polymers that can self-assemble into a core-shell structure due to the aggregation of the hydrophobic moieties when they are in an aqueous environment. EP3753966A1 discloses an amphiphilic block copolymer which includes a hydrophilic chain segment, a hydrophobic chain segment, and a linker for linking the hydrophilic chain segment to the hydrophobic chain segment. The linker contains an unsaturated structure to enhance the interaction between the poorly soluble drug (SN-38) and the copolymer.

Polymeric structures that can be self-assembled into micelles comprising in their structure encapsulated SN-38 have also been disclosed. For instance, A. Buckin et al., in “Amphiphilic Polymeric Nanoparticles Modified with a Protease-Resistant Peptide Shuttle for the Delivery of SN-38 in Diffuse Intrinsic Pontine Glioma” ACS Applied Nano Materials 2021, vol. 4 (2), pp. 1314-1329, disclose SN-38-loaded polymeric nanoparticles of an amphiphilic chitosan (CS)-g-poly(methyl methacrylate)-poly(acrylic acid) copolymer that were surface-modified with a peptide shuttle that improves transport across the BBB.

CN102060991A1 describes an amphiphilic drug precursor of 7-ethyl-10-hydroxycamptothecin, whose OH at 10- or 20-position is linked with hydrophilic groups (such as PEG-200-2000) and that can form micelles.

Ri, Masaki et al., in “A phase I/II study for dose-finding, and to investigate the safety, pharmacokinetics and preliminary efficacy of NK012, an SN-38-incorporating macromolecular polymeric micelle, in patients with multiple myeloma “Internal Medicine (Tokyo, Japan) (2018), vol. 57(7), pp. 939-946, disclose SN-38-releasing polymeric micelles constructed by covalently attaching SN-38 to the block copolymer PEG-pGlu, followed by self-assembly of amphiphilic block copolymers in aqueous media.

CN110124052A discloses a conjugate of a polyethylene glycol monomethyl ether coupled E-selectin peptide ligand, and an anti-tumor drug such as camptothecin, hydroxycamptothecin, SN-38, paclitaxel, docetaxel, dasatinib, gemcitabine, doxorubicin or podophyllotoxin, that can self-assemble into nanoparticle in an aqueous solution.

Finally, Lei, Fan et al:, in Nanoscale platform for delivery of active IRINOX to combat pancreatic cancer Journal of Controlled Release 2021, vol. 330, pp. 1229-1243, disclose a crosslinked micelles that were prepared using amphiphilic PEG-b-poly(L-glutamic acid)/SN-38 conjugates and subsequently loaded with dichloro(1,2-diaminocyclohexane)platinum(II) (DACHPt).

Despite the efforts to delivery systems of SN-38 to allow the adequate administration and bioavailability of SN-38 for the treatment of cancer, there is still an unmet medical need of finding improved systems to efficiently administer SN-38, and even more so considering that neither irinotecan nor SN-38 can cross the BBB significantly. Thus, they have no efficacy in brain tumors with intact blood-brain barrier (BBB), such as diffuse intrinsic pontine gliomas (DIPG) and high grade gliomas (HGG), including its pediatric form (pHGG).

### SUMMARY OF INVENTION

The inventors have found that certain peptide conjugates of SN-38 in an aqueous medium form micelles by spontaneous self-assembly and are able to load free drugs into its core.

These micelles are particularly advantageous when they are loaded with SN-38 lactone and/or another anticancer drug. Unexpectedly, by the formation of micelles with these peptide conjugates of SN-38 and free SN-38 lactone, the apparent solubility of free SN-38 lactone in water at acidic pH increases at least 500 times, which is well above the state of the art.

The lactone form of SN-38 is extremely insoluble in water at acidic pH. However, acidic pH is needed to conserve SN-38 in its lactone (active) form, which undergoes a reversible and pH-dependent conversion to SN-38 carboxylate (freely water-soluble inactive form, due to the opening of the lactone ring at neutral or basic pH). The intrinsic aqueous solubility of SN-38 lactone at pH 3 has been determined as 8 μg/mL (own data). The reported solubility of SN-38 in water, pH 3.5 and 7.4 buffers, is 11-38 μg/ml, 7.2 μg/ml, and 36 μg/ml, respectively (see Zhang et al., “Development and characterization of a novel liposome-based formulation of SN-38” International Journal of Pharmaceutics, 2004, vol. 270, pp. 93-107).

Conversely, in the present invention, the apparent solubility of free SN-38 lactone in water at acidic pH is at least 4 mg/mL, so, the solubility increases of at least 500 times, whereas the SN-38 liposomes disclosed in Zhang et al achieves a SN-38 apparent solubility of 0.111 mg/mL. This is achieved without the need of pharmaceutical excipients or lipid components, and it surpasses in more than 40 times the solubility of the best liposome formulation of SN-38 reported in the previous state of the art.

Due to the insolubility of SN-38 lactone, in the clinical practice SN-38 is administered as irinotecan, its water-soluble prodrug, approved for several cancer indications. Upon administration in the bloodstream, irinotecan releases SN-38 lactone as its active metabolite, due to the action of the carboxylesterase enzymes. Conversion of irinotecan to SN-38 is low in humans, however, due to limited expression of these enzymes. This would explain why irinotecan is very potent in animal models rich in carboxylesterase (mice) while less potent in humans (carboxylesterase-low). Advantageously, the present invention surpasses the need of carboxylesterases because it carries SN-38 lactone free.

Finally, unlike irinotecan and SN-38, the micelles of the present invention cross the blood brain barrier (BBB) and achieve therapeutic concentrations of SN-38 in the brain and cerebrospinal fluid (CSF), leading to therapeutic activity in DIPG and pHGG xenografts. Thus, a water-soluble brain-penetrant delivery system of an anticancer drug useful in the treatment of brain cancer is provided.

The SN-38 peptides conjugates are formed from some peptides disclosed in WO2015/001015A1 and specific linkers of a certain size that are larger than the linkers disclosed in the mentioned document. These conjugates by themselves provide in vitro an antitumoral activity up to 100 times superior to the in vitro activity of irinotecan. They already present high solubility in water, and an activity by their own much higher than the activity of irinotecan in vitro. The SN-38-loaded micelles of the present invention show antitumoral activity against several cell lines from brain and extracranial cancers and are more active that the peptide conjugates alone.

The micelles are prepared from the peptide conjugates of SN-38 by their dissolution in water at pH<7, preferably <3, by spontaneous micellization. Such micelle acidic solutions can neutralize a basic solution containing free SN-38 carboxylate at concentrations of up to 25 mg/mL. Upon acidification of the basic pH, the precipitation of free SN-38 lactone due to the pH change leads to inter-molecular interactions between free SN-38 lactone molecules and the SN-38 molecule conjugated in the peptide conjugate of SN-38. Micellar dispersions of peptide conjugates of SN-38 containing free SN-38 lactone can be easily filtered through 0.22 and 0.45 μm pore filters without significant loss of free SN-38 lactone. The final system is composed of drug-drug cocrystals comprised of two forms of SN-38 lactone, a free form and a conjugated form. Under such conditions, free SN-38 lactone does not form microcrystals or larger solid structures and remains apparently soluble at concentrations up to 4 mg/ml.

Micellar dispersions of these SN-38 bound peptide conjugates containing free SN-38 lactone are in the nanometric size scale. They can be used to load into its micellar core free SN-38. Thus, for instance, as it is illustrated in Example 15 the loading is increased in this example from the current 21.5% SN-38 loading (w/w), already included in the conjugated product, up to 35% w/w SN-38 in the final micelle product, including the free SN-38 lactone loaded in the micelles. The size of this micelle is around 40 nm at a concentration of 11 mM (20 mg/mL) without free SN-38 lactone and around 100 nm upon encapsulation of free SN-38 lactone, due to the effect of loading the micelles with free SN-38 lactone.

Thus, a first aspect of the present invention relates to micelles comprising a peptide conjugate of SN-38 and one or more free therapeutic active agents which anticancer activity, wherein: the micelle is a core-shell structure comprising an inner core and an external shell wherein the free therapeutic active agent is loaded in the inner core and the peptide conjugate of SN-38 forms the external shell; the peptide conjugate of SN-38 is a compound of formula (I) or a pharmaceutically acceptable salt thereof,

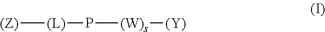

wherein: Z is a radical of the pharmaceutical active ingredient SN-38 or a pharmaceutically acceptable salt thereof, wherein the pharmaceutical active ingredient SN-38 has formula (II), and wherein Z is attached to a linker L independently by only one of the two hydroxyl groups (a) or (b) of the pharmaceutical active ingredient;

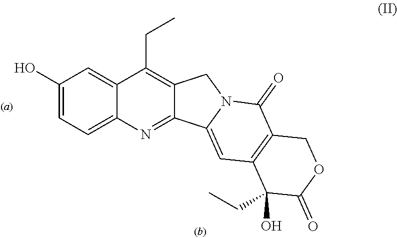 [SMILES: CCc1c2c(nc3ccc(O)cc13)-c1cc3c(c(=O)n1C2)COC(=O)[C@]3(O)CC]

L is a linker which is a biradical composed from 2 to 8 biradicals L′ and has the formula: -L′ₐ-(L′_{b})ₙ-L′_{c};
Lₐ′ is a biradical selected from the group consisting of: —C(═O)—(CH₂)ᵣ—C(═O)—; —C(═O)—(CH₂)ᵣ—NH—; —C(═O)—(CH₂)ᵣ—S—; —C(═O)—(CH₂)ᵣ—O—; —C(═O)—NH—(CH₂)ᵣC(═O)—; —C(═O)—NH—(CH₂)ᵣ—NH—; —C(═O)—NH—(CH₂)ᵣ—S—; —C(═O)—NH—(CH₂)ᵣ—O—; —(CH₂)ᵣC(═O)—; —(CH₂), NH—; —(CH₂), S—; —(CH₂), O—; —Si(R₁)(R₂)—(CH₂), NH—; —Si(R₁)(R₂)—(CH₂), C(═O)—; —Si(R₁)(R₂)—(CH₂)ᵣ—O—; —Si(R₁)(R₂)—(CH₂)ᵣ—S—; —SO₂—(CH₂)ᵣ—NH—; —SO₂—(CH₂)ᵣ—C(═O)—; —SO₂—(CH₂)ᵣ—O—; —SO₂—(CH₂)ᵣ—S—; —P(═O)(OR₁)—O—(CH₂)ᵣ—NH—; —P(═O)(OR₁)—O—(CH₂)ᵣC(═O)—; —P(═O)(OR₁)—O—(CH₂)ᵣO—; —P(═O)(OR₁)—O—(CH₂)ᵣ—S—; —CH(OH)—(CH₂)ᵣ—NH—; —CH(OH)—(CH₂)ᵣC(═O)—; —CH(OH)—(CH₂)ᵣ—O—; —CH(OH)—(CH₂)ᵣ—S—;

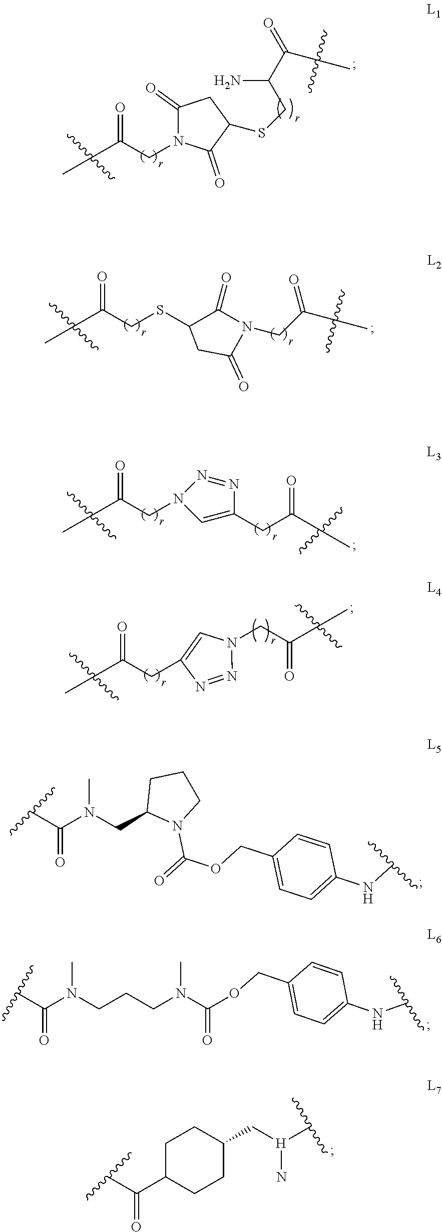

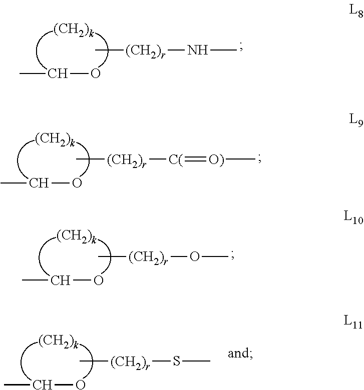

the substituent in any of L₈-L₁₁ is in any position of the cycles;

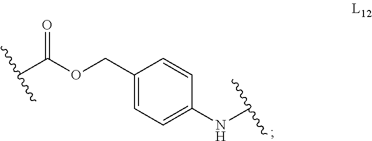 [SMILES: *Nc1ccc(COC(*)=O)cc1]

L_{b}′ is a biradical independently selected from the group consisting of: —NH—(CH₂)ᵣC(═O)—; —C(═O)—(CH₂)ᵣC(═O)—; —S—(CH₂)ᵣC(═O)—; —O—(CH₂)ᵣC(═O)—; —NH—(CH₂)ᵣ; —C(═O)—(CH₂)ᵣ; —S—(CH₂)ᵣ; —O—(CH₂)ᵣ; —NH—CH—((CH₂)ᵣ—NH₂)—C(═O)—; —S—CH₂—CH(NH₂)—C(═O)—; —(CH₂)ᵣC(═O)—; —(CH₂)ᵣ—O—; —(CH₂)ᵣ—NH—; —(CH₂)ᵣ—S—; —C(═O)—(CH₂)ᵣ—NH—; —C(═O)—(CH₂)ᵣO—; —C(═O)—(CH₂)ᵣ—S—; —NH—(CH₂)ᵣ—O—; —NH—(CH₂)ᵣ—NH—; —NH—(CH₂)ᵣ—S—; L₁; L₂; L₃; L₄; and combinations thereof;
L_{c}′ is a biradical selected from the group consisting of: —NH—(CH₂)ᵣC(═O)—; —NH—CH—((CH₂)ᵣ—NH₂)—C(═O)—; —C(═O)—(CH₂)ᵣC(═O)—; —S—(CH₂)ᵣC(═O)—; —S—CH₂—CH(NH₂)—C(═O)—; —O—(CH₂)ᵣC(═O)—; —(CH₂)ᵣC(═O)—; L₁, L₂; L₃; L₄

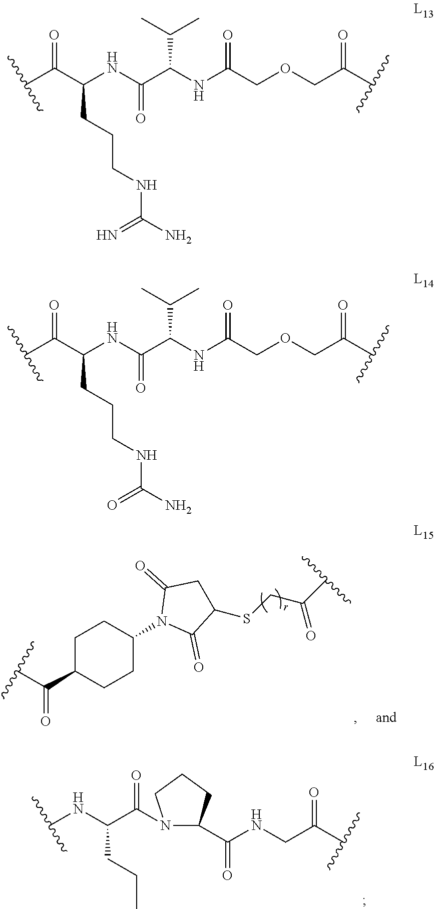

P is a biradical of a peptide selected from the group consisting of: (a) a peptide which comprises the amino acid sequence X₁KAPETALX₂ with an intrapeptide bond between the X₁ and X₂ which is an amide bond; wherein X₁ is selected from the group consisting of Dap (2,3-diaminopropionic acid) and Dab (2,4-diaminobutanoic acid); and X₂ is selected from the group consisting of D (aspartic acid) and E (glutamic acid); i.e.

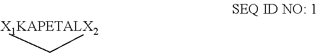

For the amino acid Dap, the code Dap and Dpr have been equally used herein;
(b) a peptide having 12-20 amino acids residues in length having at least an intrapeptide bond which is a disulfide or diselenide bond, and comprises an amino acid sequence which is: X₃KAPETALX₄AAA; having at least an intrapeptide disulfide or diselenide bond between X₃ and X₄, wherein X₃ and X₄ are equal and are selected from the group consisting of C (cysteines), Sec (selenocysteines), and Pen (penicillamines); i.e.

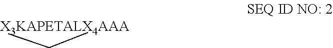

(c) a peptide having 9-11 amino acids residues in length having at least an intrapeptide bond which is a disulfide or diselenide bond and consists of an amino acid sequence selected from the group consisting of X₅KAPETALX₆; X₅KAPETALX₆A; and XsKAPETALX₆AA having at least an intrapeptide disulfide or diselenide bond between X₅ and X₆; wherein X₅ and X₆ are equal and are selected from the group consisting of C (cysteines), Sec (selenocysteines), and Pen (penicillamines), i.e.

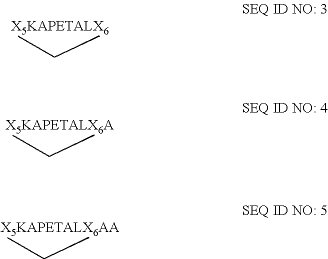

(d) a peptide which has 16 amino acid residues and comprises the amino acid sequence X₇NX₈KAPETALXsAAAX₁₀H with an intrapeptide disulfide or diselenide bond between the X₇ and X₉, and between X₈ and X₁₀; wherein X₇-X₁₀ are independently selected from the group consisting of C (cysteines), Sec (selenocysteines), and Pen (penicillamines); provided that X₇ and X₉ are equal, and X₈-X₁₀ are equal; i.e.

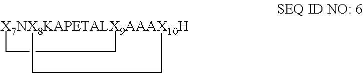

and (e) peptide which comprises the amino acid sequence X₁KAPETALX₂ wherein X₁ is selected from the group consisting of Dap and Dab; and X₂ is selected from the group consisting of D (aspartic acid) and E (glutamic acid) (SEQ ID NO:7) being a linear peptide;
W is a biradical selected from the group consisting of —NH—(CH₂)ᵣC(═O)—, and
—NH—CH((CH₂)ᵣ—NH₂)—C(═O)—; Y is a radical is selected from the group consisting of
—NH₂, —OH, —OR₃, and —NHR₃; s is an integer independently selected from 0 to 1; n is an integer from 0 to 6; r is an integer independently selected from 1 to 5; k is an integer from 5 to 8; R₁ and R₂ are independently selected from an (C₁-C₆)-alkyl; and R₃ is a radical selected from the group consisting of (C₁-C₆)-alkyl;
Lₐ′ is attached to the radical Z through a bond which is selected from the group consisting of an ester, ether, urethane, silyl ether, sulphonate, phosphate, ketal, hemiketal, carbonate, and carbamate bond, the bond being formed between the C═O, SO₂, Si, P, CH or CH₂ groups on the left side of the draw Lₐ′ formulas and one of the hydroxyl groups of the SN-38;
when n=0, Lₐ′ is attached to the radical L_{c}′ through a chemically feasible bond which is selected from the group consisting of amine, amide, ether, thioether, disulfide, ester, and thioester, the bond being formed between the functional groups on the right side of the draw Lₐ′ formulas and the functional groups of the left side of the L_{c}′ formulas;
when n=1, Lₐ′ is attached to the radical L_{b}′ through a chemically feasible bond which is selected from the group consisting of amine, amide, ether, thioether, disulfide, ester, and thioester, the bond being formed between the functional groups on the right side of the draw Lₐ′ formulas and the functional groups on the left side of the L_{b}′ formulas; and L_{b}′ is attached to the radical L_{c}′ through a chemically feasible bond which is selected from the group consisting of amine, amide, ether, thioether, disulfide, ester, and thioester, the bond being formed between the functional groups on the right side of the draw L_{b}′ formulas and the functional groups on the left side of the draw L_{c}′ formulas;
when n is higher than 1, L_{b}′ are equal or different and are attached among them through a chemically feasible bond selected from the group consisting of amine, amide, ether, thioether, disulfide, ester, and thioester; being one L_{b}′ terminal attached to Lₐ′ through a chemically feasible bond which is selected from the group consisting of amine, amide, ether, thioether, disulfide, ester, and thioester, the bond being formed between the functional groups on the right side of the draw Lₐ′ formulas and the functional groups of the left side of the draw L_{b}′ formulas; and being another L_{b}′ terminal attached to L_{c}′ through a chemically feasible bond which is selected from the group consisting of amine, amide, ether, thioether, disulfide, ester, and thioester, the bond being formed between the functional group on the right side of the draw L_{b}′ formulas and the functional group on the left side of the draw L_{c}′ formulas;
L_{c}′ is attached to the to the biradical P through an amide bond formed with the carbonyl group on the right side of the draw L_{c}′ formulas and an amino group of the first amino acid of the peptide sequence P;
when s=0, P is directly attached to Y through an amide, carboxylic acid or ester bond, the bond being formed between the C═O of the C-terminal of the last amino acid of the sequence P, and the radical Y which is —NH₂, —OH, —OR₃, or —NHR₃; and
when s=1, P is attached to a radical W through an amide bond formed with a C═O of the C-terminal of the last amino acid of the sequence P, the bond being formed between the functional groups on the left side of the draw W formulas and the functional groups (C═O) of the C-terminal of the last amino acid of the sequence P on the right side of the draw sequence; and W is attached to Y as follows: —C(═O)—NH—(CH₂)ᵣC(═O)—Y, or —C(═O)—NH—CH((CH₂)ᵣ—NH₂)—C(═O)—Y.

The lines between two amino acids of the sequences above or below represent the intrapeptide bond between the side chains of the two amino acids. In a particular embodiment, the lines between two amino acids of the sequences above or below represent the intrapeptide bond between the side chains of the two amino acids.

A second aspect of the present invention relates to a pharmaceutical composition comprising a therapeutically effective amount of micelles as defined above, together with appropriate amounts of pharmaceutically acceptable carriers or excipients.

A third aspect of the present invention relates to micelles as defined above, for use as a medicament.

A four aspect of the present invention relates to micelles as defined above, for use in the treatment of cancer in a mammal, including a human.

A fifth aspect of the present invention relates to micelles as defined above for use in the treatment of cancer, wherein the compound of formula (I) is for use in combination therapy with a chemotherapeutic agent.

### BRIEF DESCRIPTION OF DRAWINGS

FIG. 1 shows an scheme of the manufacturing process of the new water-soluble SN-38 lactone micellar products.

FIG. 2 is a photograph obtained immediately after the preparation of product G2B-002-20-9 or products containing 2 mg/mL load of free SN-38 lactone in the absence of G2B-001 micelles, consisting of only vehicle, or irinotecan 20 mg/mL in vehicle. The product G2B-002-20-9 was not turbid, while the products manufactured with vehicle or irinotecan 20 mg/mL in vehicle showed turbidity corresponding to insoluble SN-38 lactone crystals. Left: vehicle containing 2 mg/mL SN-38 lactone; center: G2B-002-20-9; right: irinotecan 20 mg/mL in vehicle, containing 2 mg/mL SN-38 lactone.

FIG. 3 is a photograph obtained immediately after the preparation of product G2B-006-20-9 or product containing 2 mg/mL load of free SN-38 lactone in the absence of G2B-003 micelles, consisting of only vehicle. The product G2B-006-20-9 was not turbid, while the product manufactured with vehicle showed turbidity corresponding to insoluble SN-38 lactone crystals. Left: G2B-006-20-9; right: vehicle.

FIG. 4 is a photograph of the product G2B-002-20-9, obtained at different times. The product remained without turbidity for 24 weeks, stored at 4° C. Left: G2B-002-20-9 immediately after preparation; center: G2B-002-20-9 one week after preparation; right: G2B-002-20-9 24 weeks after preparation.

FIG. 5 is a photograph of the product G2B-006-20-9, obtained at different times. The product remained without turbidity for 24 weeks, stored at 4° C. Left: G2B-006-20-9 immediately after preparation; center: G2B-002-20-9 six weeks after preparation; right: G2B-006-20-9 24 weeks after preparation.

FIG. 6 is a photograph of products detailed in Table 4, containing camptothecin (CPT) in the concentration range 1-0.25 mg/mL.

FIG. 7 is a photograph of products detailed in Table 5, containing mixtures of SN-38 lactone and camptothecin (CPT) at concentrations 1 and 0.5 mg/mL, in the presence of G2B-001, or in its absence (vehicle).

FIG. 8 is a comparative of the antiproliferative activity of G2B-002-20-9, free SN-38 and irinotecan, against cancer cell line HSJD-DIPG-007. Values in dots represent means and SD from three replicates at compound concentration

FIG. 9 is a comparative of the antiproliferative activity of G2B-002-20-9, free SN-38 and irinotecan, against cancer cell line HSJD-DMG-001. Values in dots represent means and SD from three replicates at compound concentration

FIG. 10 is a comparative of the antiproliferative activity of G2B-002-20-9, free SN-38 and irinotecan, against cancer cell line HSJD-GBM-001. Values in dots represent means and SD from three replicates at compound concentration

FIG. 11 is a comparative of the antiproliferative activity of G2B-002-20-9, free SN-38 and irinotecan, against cancer cell line RH4. Values in dots represent means and SD from three replicates at compound concentration.

FIG. 12 is a comparative of the antiproliferative activity of G2B-002-20-9, free SN-38 and irinotecan, against cancer cell line A673. Values in dots represent means and SD from three replicates at compound concentration.

FIG. 13 shows the Kaplan-Meier survival curves obtained in each of the groups of HSJD-DIPG-007-bearing mice.

FIG. 14 shows Kaplan-Meier survival curves obtained in each of the groups of HSJD-GBM-001-bearing mice.

FIG. 15 shows tumor growth (% of tumor volume at day 1 of treatment) of mice bearing subcutaneous PDX treated with saline control, irinotecan 10 mg/kg and G2B-002 versions at dosages 1 and 10 mg/kg of SN-38 lactone. Each dot represents the tumor growth of one individual PDX model.

FIG. 16 shows the concentration-time data of SN-38 lactone in mouse retinae following the intravenous administration of G2B-002-20-9 at doses 10 mg/kg and 1 mg/kg of SN-38 lactone, or irinotecan at 10 mg/kg. Dots are individual data and lines connect data means at each time point.

FIG. 17 shows transport in Human in vitro BBB model of different micelles formed by compound Ia and SN-38 compared with SN-38 and SN-38 peptide conjugate (G2B-001).

### DETAILED DESCRIPTION OF THE INVENTION

All terms as used herein in this application, unless otherwise stated, shall be understood in their ordinary meaning as known in the art. Other more specific definitions for certain terms as used in the present application are as set forth below and are intended to apply throughout the description and claims

Unless otherwise stated, the amino acids cited herein are L-amino acids. The 1-letter code and the 3-letter code have been used indistinctly. For the following amino acids, the following abbreviations have been used: diaminopropionic acid (Dap), Diaminobutiric (Dab), Selenocystein (Sec) and Penicillamine (Pen). In the context of the present invention penicillamine only embraces D-penicillamine.

The term “apparent solubility” is the aqueous solubility of free SN-38 lactone measured upon inclusion in the aqueous micellar dispersion of peptide conjugates of SN-38.

The term “inner core” as it applies to a micelle of the present invention refers to the center of the micelle formed by the peptide conjugate of SN-38.

The term “external shell” as it applies to a micelle of the present invention refers to the layer formed by the peptide conjugate of SN-38.

As used herein, the terms “drug” and “therapeutic agent” are used interchangeably.

The term “drug loaded” and “encapsulated” are used interchangeably. In accordance with the present invention a “drug loaded” micelle refers to a micelle having a drug or therapeutic agent situated within the core of the micelle.

Where in the present invention a numerical interval is used, this includes the values of the extremes of the interval. In particular, as used herein, the term “comprised between” to refer to a range of values including the end points of the range.

Unless otherwise stated, all percentages mentioned herein are expressed in weight with respect to the total weight of the product, provided that the sum of the amounts of the components is equal to 100%.

Compound (Ia) is also named as G2B-001. Compound (Ib) is also named as G2B-001 linear. Compound (Ic) is also named as G2B-003. Compound (Id) is also named as G2B-004. Compound (Ie) is also named as G2B-005.

As an example of the nomenclature used, in G2B-002-20-9, the percentage weight/weight (% w/w) of free SN-38 in the final formulation is 9% w/w, i.e., free SN-38/(SN-38+G2B-001)*100=9% and final concentrations of 20 mg/mL for G2B-001(see also Example 15).

The word “comprise” for the purposes of the present invention encompasses the case of “consisting of”.

As mentioned above, it is part of the invention micelles of SN-38 bound peptide conjugates loaded with at least a drug which has anticancer activity. In a particular embodiment the micelles of SN-38 bound peptide conjugates are loaded with a camptothecin. In another particular embodiment the camptothecin is selected from the group consisting of SN-38, camptothecin (CPT), or topotecan. In a particular embodiment, the drug in the inner core is SN-38 lactone.

The peptide conjugates of SN-38 used to prepare the micelles of the present invention mentioned above may be in the form of pharmaceutically acceptable salts. The term “pharmaceutically acceptable salts” used herein encompasses any salt formed from pharmaceutically acceptable non-toxic acids or bases including inorganic or organic acids or bases. There is no limitation regarding the salts, except that if used for therapeutic purposes, they must be pharmaceutically acceptable. As some of the compounds of formula (I) are basic compounds, salts may be prepared from pharmaceutically acceptable non-toxic acids, including inorganic and organic acids. Such acids include, for instance, chlorhydric, acetic, benzenesulfonic, benzoic, camphorsulfonic, citric, ethansulfonic, fumaric, gluconic, glutamic, hydrobromic, hydrochloric, lactic, maleic, malic, mandelic, methanesulfonic, phosphoric, succinic, sulfuric, tartaric, p-toluensulfonic acid, and the like.

In another particular embodiment, the micelles are those where in the peptidic conjugate of SN-38 of formula (I), Z is attached to a linker L by the hydroxyl group (b) of the pharmaceutical active ingredient. In another particular embodiment, the micelles are those where in the peptidic conjugate of SN-38 of formula (I), Z is attached to a linker L by the hydroxyl group (a) of the pharmaceutical active ingredient.

In another particular embodiment, the micelles are those where in the peptidic conjugate of SN-38 of formula (I), P is a biradical of a peptide selected from the group consisting of:
(a) a peptide which comprises the amino acid sequence DapKAPETALD with an intrapeptide bond between the Dap and D which is an amide bond, that is

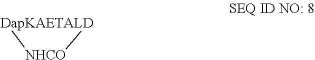

(b) a peptide having 9-20 amino acids residues in length having at least an intrapeptide bond which is a disulfide bond, and comprises an amino acid sequence which is: CKAPETALCAAA having at least an intrapeptide disulfide bond between cysteines 1 and 9, that is

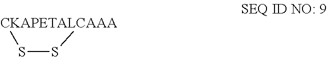

(c) a peptide having 9-11 amino acids residues in length having at least an intrapeptide bond which is a disulfide bond and consists of an amino acid sequence selected from the group consisting of CKAPETALC; CKAPETALCA; and CKAPETALCAA having at least an intrapeptide disulfide bond between cysteines 1 and 9, that are: SEQ ID NO:10: CKAPETALC

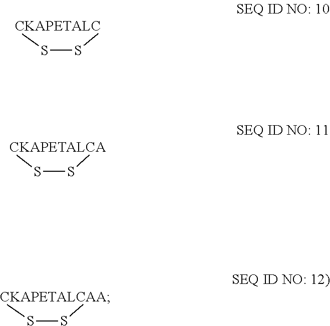

(d) a peptide which has 16 amino acid residues and comprises the amino acid sequence CNCKAPETALCAAACH with an intrapeptide disulfide bond between the first and third cysteine which are cysteines 1 and 11, and between the second and the fourth cysteine which are cysteine 3 and 15, that is,

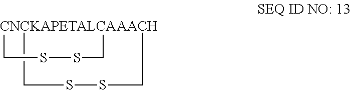

(e) a peptide which comprises the amino acid sequence DapKAPETALD (SEQ ID NO:14), i.e., linear peptide.

In sequences SEQ ID NO:8 to SEQ ID NO:13 in which specific amino acids have been selected for X₁-X₁₀ the specific intrapeptide bond has been drawn in the sequence.

In another particular embodiment, the peptides of the present invention are those having one intrapeptide bond. In another particular embodiment, the peptides of the present invention are those having to two intrapeptide bonds.

In another particular embodiment, the micelles are those where in the peptidic conjugate of SN-38 of formula (I), P is a biradical of a peptide selected from the group consisting of: (a) the peptide having the amino acid sequence DapKAPETALD with an intrapeptide bond between the Dap and D which is an amide bond (SEQ ID NO:8); (b) the peptide having the amino acid sequence CKAPETALC having at least an intrapeptide disulfide bond between cysteines in position 1 and 9 (SEQ ID NO:10); (c) the peptide having the amino acid sequence DapKAPETALD (SEQ ID NO:14), i.e linear peptide. In another particular embodiment, the micelles are those where in the peptidic conjugate of SN-38 of formula (I), P is a biradical of the peptide DapKAPETALD with an intrapeptide bond between the Dap and D which is an amide bond (SEQ ID NO: 8).

In another particular embodiment, the micelles are those where in the peptidic conjugate of SN-38 of formula (I), Lₐ′ is a biradical selected from the group consisting of: —C(═O)—(CH₂)ᵣC(═O)—, —C(═O)—(CH₂)ᵣ—NH—, —C(═O)—(CH₂)ᵣ—S—; —C(═O)—(CH₂)ᵣ—O—; —C(═O)—NH—(CH₂)ᵣ C(═O)—; L₁, L₂, L₃, L₄, L₅, L₆, L₇, and L₁₂.

In another particular embodiment, in combination of any of the particular embodiments above or below, the micelles are those where in the peptidic conjugate of SN-38 of formula (I), L is a linker which is a biradical composed from 3 to 8 biradicals and n is an integer from 1 to 6. In another particular embodiment, in combination of any of the particular embodiments above or below, the micelles are those where in the peptidic conjugate of SN-38 of formula (I), L is a linker which is a biradical composed from 5 to 8 biradicals. In another particular embodiment, in combination of any of the particular embodiments above or below, the micelles are those where in the peptidic conjugate of SN-38 of formula (I), L is a linker which is a biradical composed from 6 to 8 biradicals. In another particular embodiment, in combination of any of the particular embodiments above or below, the micelles are those where in the peptidic conjugate of SN-38 of formula (I), L is a linker which is a biradical composed from 6 to 7 biradicals. In another particular embodiment, in combination of any of the particular embodiments above or below, the micelles are those where in the peptidic conjugate of SN-38 of formula (I), L is a linker which is a biradical composed from 6 biradicals.

In another particular embodiment, the micelles are those where in the peptidic conjugate of SN-38 of formula (I), Lₐ′ is L₃ of formula below and L_{c}′ is —C(═O)—(CH₂)ᵣC(═O)—.

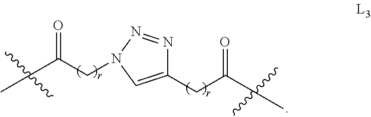

In another particular embodiment, the micelles are those where in the peptidic conjugate of SN-38 of formula (I), L_{b}′ is selected from the group consisting of —NH—(CH₂)ᵣ—O—, —(CH₂)ᵣ O—; and —(CH₂)ᵣ—NH—, and combinations thereof. In another particular embodiment, the micelles are those where in the peptidic conjugate of SN-38 of formula (I), Lₐ′ is L₃ of formula above, L_{b}′ is selected from the group consisting of —NH—(CH₂)ᵣ—O—, —(CH₂)ᵣ—O—; and —(CH₂)ᵣ—NH—, and combinations thereof, and L_{c}′ is —C(═O)—(CH₂)ᵣC(═O)—.

In another particular embodiment, in combination of any of the particular embodiments above or below, the micelles are those where in the peptidic conjugate of SN-38 of formula (I), L is a linker which is a biradical composed from 2 biradicals, and n=0. In another particular embodiment, in combination of any of the particular embodiments above or below, the micelles are those where in the peptidic conjugate of SN-38 of formula (I), Lₐ′ is L₁₂ and L′ is L₁₃. In another particular embodiment, in combination of any of the particular embodiments above or below, the micelles are those where in the peptidic conjugate of SN-38 of formula (I), Lₐ′ is —C(═O)—NH—(CH₂)ᵣC(═O)— and L_{c}′ is L₁s.

In another particular embodiment, in combination of any of the particular embodiments above or below, the micelles are those where in the peptidic conjugate of SN-38 of formula (I), Lₐ′ is attached to the radical Z through a bond which is an ester bond formed with the C═O group on the left side of the draw Lₐ′ formulas, and to the radical L_{b}′ through a chemically feasible bond which is selected from the group consisting of amine, amide, ether, thioether, disulfide, ester, and thioester, formed with the functional groups on the right side of the draw formulas. In another particular embodiment, in combination of any of the particular embodiments above or below, the micelles are those where in the peptidic conjugate of SN-38 of formula (I), Lₐ′ is attached to the radical Z through a bond which is an ester bond formed with the C═O group on the left side of the draw Lₐ′ formulas, and to the radical L_{b}′ through a chemically feasible bond which is amide bond, formed with the functional groups on the right side of the draw formulas. In another particular embodiment, in combination of any of the particular embodiments above or below, the micelles are those where in the peptidic conjugate of SN-38 of formula (I), Lₐ′ is attached to the radical Z through a bond which is a carbonate or a carbamate bond formed with the C═O group on the left side of the draw Lₐ′ formulas, and to the radical L_{b}′ through a chemically feasible bond which is amide bond (NH—CO or CO—NH), formed with the functional groups on the right side of the draw formulas.

In another particular embodiment, in combination of any of the particular embodiments above or below, the micelles are those where in the peptidic conjugate of SN-38 of formula (I), L_{b}′ forms the chemically feasible bond with the radical Lₐ′ with the functional groups on the left side of the L_{b}′ drawn formulas; and L_{b}′ is attached to the radical L_{c}′ through a chemically feasible bond which is selected from the group consisting of amine, amide, ether, thioether, disulfide, ester, and thioester, formed with the functional groups on the right side of the L_{b}′ drawn formulas; wherein when n is higher than 1, L_{b}′ are equal or different and are attached among them through a chemically feasible bond selected from the group consisting of amine, amide, ether, thioether, disulfide ester, and thioester; being one L_{b}′ terminal attached to Lₐ′ and the other L_{b}′ terminal attached to L_{c}′.

In another particular embodiment, in combination of any of the particular embodiments above or below, the micelles are those where in the peptidic conjugate of SN-38 of formula (I), L_{c}′ is attached to the to the biradical P through an amide bond formed with the carbonyl group on the right side of the drawn L_{c}′ formulas and an amino group of the first amino acid of the peptide sequence P, and to the radical L_{b}′ through a chemically feasible bond which is selected from the group consisting of amine, amide, ether, thioether, disulfide, ester, and thioester, formed with the functional groups on the left side of the draw formulas.

In another particular embodiment, the micelles are those where the peptidic conjugate of SN-38 of formula (I) is a compound selected from the group consisting of: compound of formula (Ia): also named as G2B-001 or SN38-linker A-MiniAp4. MiniAp4 is DapKAPETALD where Dap is 2,3-diaminopropionic acid. Both names are used interchangeably. These names have been equally used herein. Compound of formula (Ia) below or a pharmaceutically acceptable salt thereof, is a compound of formula (I) where P is DapKAPETALD with an intrapeptide bond between Dap and D, Y=CONH₂, W=0, the linker is linker A below, and the SN-38 is attached to the linker by the hydroxyl (b).

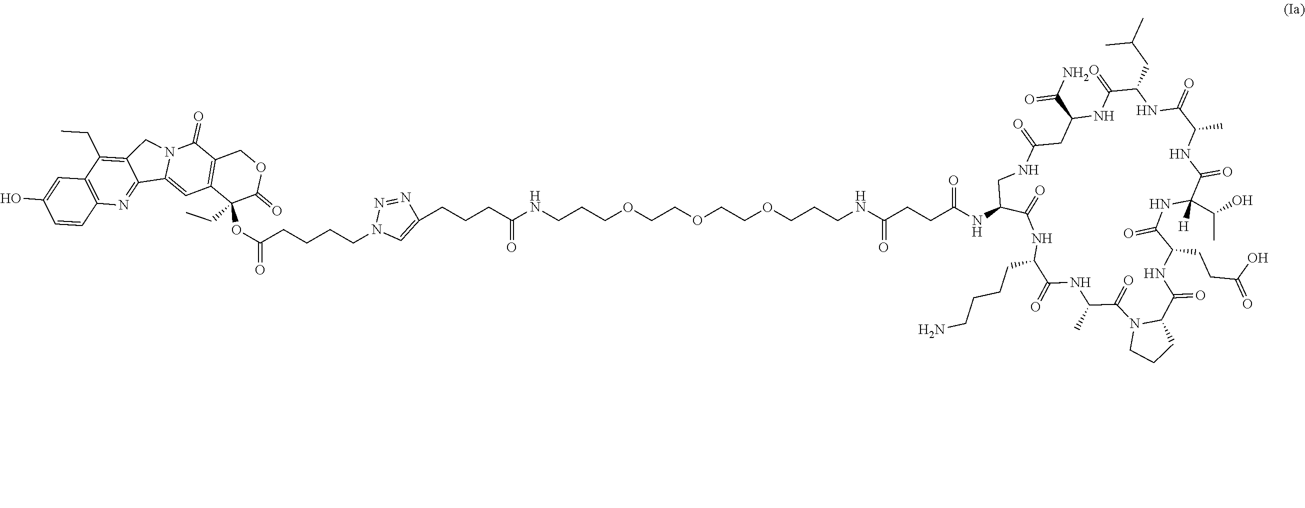 [SMILES: CCc1c2c(nc3ccc(O)cc13)-c1cc3c(c(=O)n1C2)COC(=O)[C@@]3(CC)OC(=O)CCCCn1cc(CCCC(=O)NCCCOCCOCCOCCCNC(=O)CCC(=O)N[C@H]2CNC(=O)C[C@@H](C(N)=O)NC(=O)[C@H](CC(C)C)NC(=O)[C@H](C)NC(=O)[C@H]([C@@H](C)O)NC(=O)[C@H](CCC(=O)O)NC(=O)[C@@H]3CCCN3C(=O)[C@H](C)NC(=O)[C@H](CCCCN)NC2=O)nn1]

In another particular embodiment, the compound of formula (I) is compound of formula (Ib) below, also named G2B001 linear, or a pharmaceutically acceptable salt thereof, where in formula (I) P is DapKAPETALD lineal without the intrapeptide bond between Dap and D, Y=CONH₂, W=0, the linker is linker A below, and the SN-38 is attached to the linker by the hydroxyl (b).

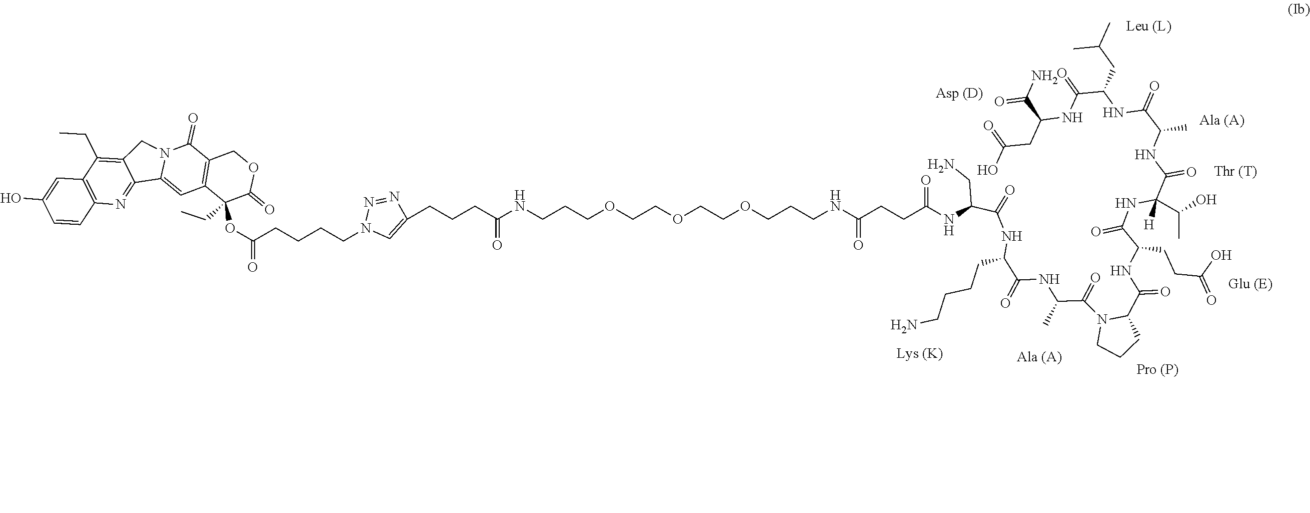 [SMILES: CCc1c2c(nc3ccc(O)cc13)-c1cc3c(c(=O)n1C2)COC(=O)[C@@]3(CC)OC(=O)CCCCn1cc(CCCC(=O)NCCCOCCOCCOCCCNC(=O)CCC(=O)N[C@@H](CN)C(=O)N[C@@H](CCCCN)C(=O)N[C@@H](C)C(=O)N2CCC[C@H]2C(=O)N[C@@H](CCC(=O)O)C(=O)N[C@H](C(=O)N[C@@H](C)C(=O)N[C@@H](CC(C)C)C(=O)N[C@@H](CC(=O)O)C(N)=O)[C@@H](C)O)nn1]

In another particular embodiment, the compound of formula (I) is compound of formula (Ic), also named G2B003, which is shown below or a pharmaceutically acceptable salt thereof, where in formula (I) P is DapKAPETALD with an intrapeptide bond between Dap and D, Y=CONH₂, W=0, the linker is linker A below, and the SN-38 is attached to the linker by the hydroxyl (a).

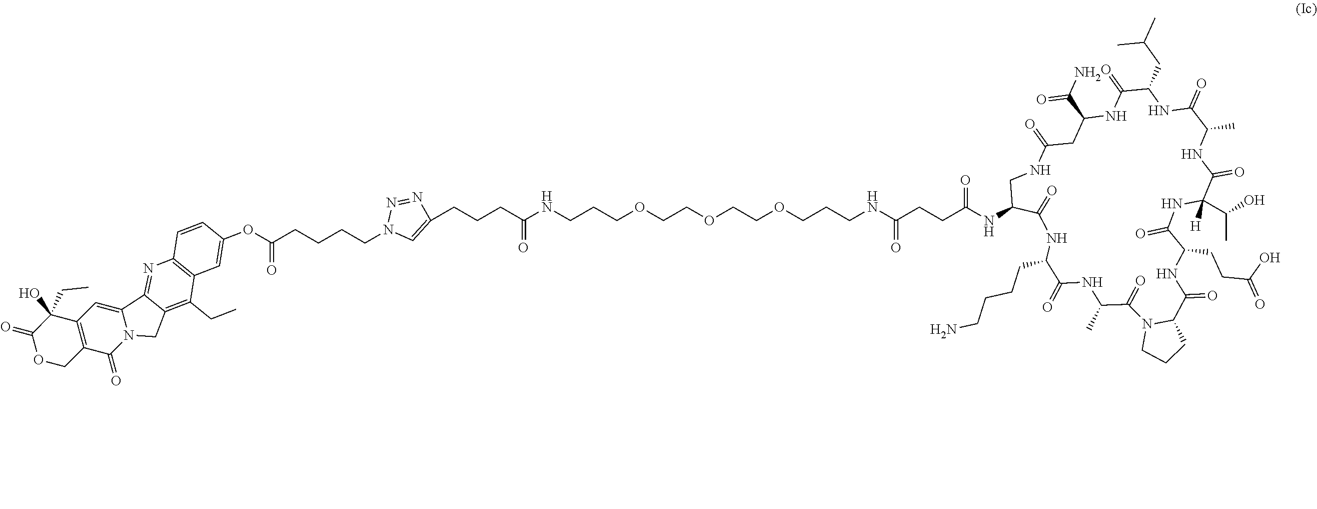 [SMILES: CCc1c2c(nc3ccc(OC(=O)CCCCn4cc(CCCC(=O)NCCCOCCOCCOCCCNC(=O)CCC(=O)N[C@H]5CNC(=O)C[C@@H](C(N)=O)NC(=O)[C@H](CC(C)C)NC(=O)[C@H](C)NC(=O)[C@H]([C@@H](C)O)NC(=O)[C@H](CCC(=O)O)NC(=O)[C@@H]6CCCN6C(=O)[C@H](C)NC(=O)[C@H](CCCCN)NC5=O)nn4)cc13)-c1cc3c(c(=O)n1C2)COC(=O)[C@]3(O)CC]

Linker A is formed by: La′: L₃ with r=4 on the right and 3 on the left:

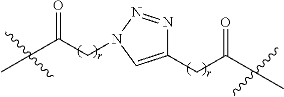

Lb′: 1 unit of biradical —NH—(CH₂)₃—O—, 2 units of biradical —(CH₂)₂—O— and 1 unit of biradical —(CH₂)₃—NH—; and L_{c}′: —C(═O)—(CH₂)₂—C(═O)—; and the biradicals are connected as in the drawing below corresponding to linker A:

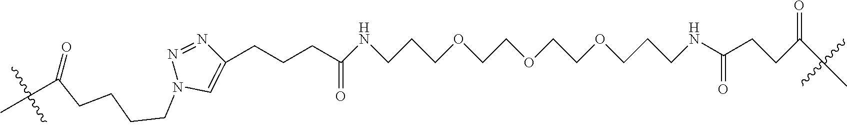 [SMILES: *C(=O)CCCCn1cc(CCCC(=O)NCCCOCCOCCOCCCNC(=O)CCC(*)=O)nn1]

In another particular embodiment, the compound of formula (I) is compound of formula (Id), also named G2B-004, which is shown below or a pharmaceutically acceptable salt thereof, where in formula (I) P DapKAPETALD with an intrapeptide bond between Dap and D, Y=CONH₂, W=0, the linker is linker C below, and the SN-38 is attached to the linker by the hydroxyl (a). Linker C is formed by La′: L₁₂ and L_{c}′: L₁₃, and n=0.

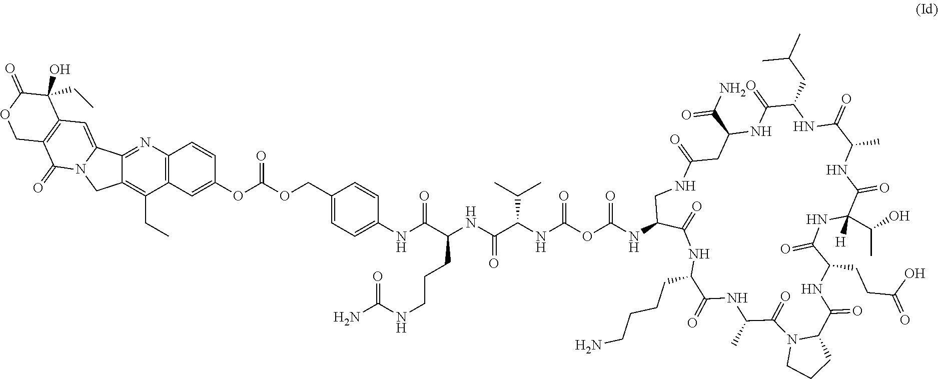 [SMILES: CCc1c2c(nc3ccc(OC(=O)OCc4ccc(NC(=O)[C@H](CCCNC(N)=O)NC(=O)[C@@H](NC(=O)OC(=O)N[C@H]5CNC(=O)C[C@@H](C(N)=O)NC(=O)[C@H](CC(C)C)NC(=O)[C@H](C)NC(=O)[C@H]([C@@H](C)O)NC(=O)[C@H](CCC(=O)O)NC(=O)[C@@H]6CCCN6C(=O)[C@H](C)NC(=O)[C@H](CCCCN)NC5=O)C(C)C)cc4)cc13)-c1cc3c(c(=O)n1C2)COC(=O)[C@]3(O)CC]

In another particular embodiment, the compound of formula (I) is compound of formula (Ie) below or a pharmaceutically acceptable salt thereof, where in formula (I) P is DapKAPETALD with an intrapeptide bond between Dap and D, Y=CONH₂, W=O, the linker is linker D below, and the SN-38 is attached to the linker by the hydroxyl (a).

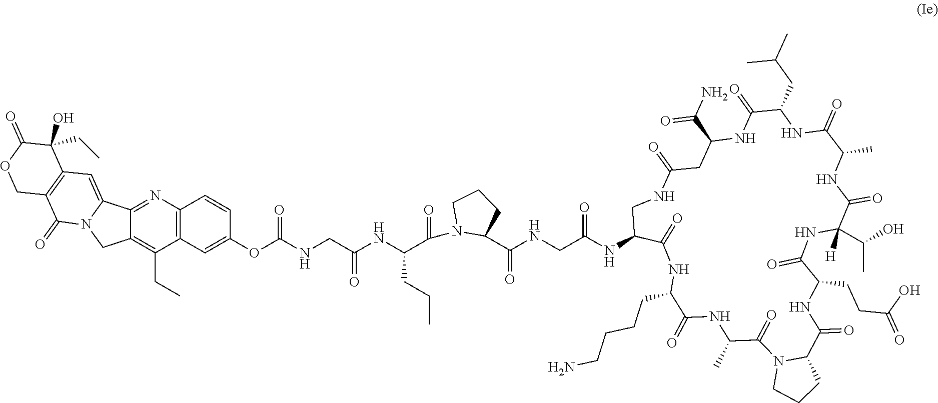 [SMILES: CCC[C@H](NC(=O)CNC(=O)Oc1ccc2nc3c(c(CC)c2c1)Cn1c-3cc2c(c1=O)COC(=O)[C@]2(O)CC)C(=O)N1CCC[C@H]1C(=O)NCC(=O)N[C@H]1CNC(=O)C[C@@H](C(N)=O)NC(=O)[C@H](CC(C)C)NC(=O)[C@H](C)NC(=O)[C@H]([C@@H](C)O)NC(=O)[C@H](CCC(=O)O)NC(=O)[C@@H]2CCCN2C(=O)[C@H](C)NC(=O)[C@H](CCCCN)NC1=O]

This compound is also named G2B-005; Linker D is formed by La′: —C(═O)—NH—(CH₂)ᵣ C(═O)— with r=1, Lb′: L₁₅ and n=0.

The micelles of the present invention are formed in an aqueous medium. Thus, the micelles of the present invention can be in form of micellar aqueous dispersion.

Micelle peptide conjugates of SN-38 containing free therapeutic agent as defined above are obtainable by: a) spontaneous self-assembly of a peptide conjugate of SN-38 as defined above in water at pH<7; b) contacting the micellar acidic solution with a basic solution containing free SN-38 carboxylate at concentrations of up to 25 mg/ml; and c) Optionally freeze-drying the micellar acidic solution.

In step b) the initial pH of the mixture is acid pH<7 and SN-38 carboxylate converts to SN-38 lactone, which forms inter-molecular interactions with the SN-38 molecule conjugated in the peptide conjugate of SN-38. In a particular embodiment, the spontaneous self-assembly of a peptide conjugate of SN-38 as defined above is carried out in water at pH<5. In a particular embodiment, the spontaneous self-assembly of a peptide conjugate of SN-38 as defined above is carried out in water at pH<3. In a particular embodiment, the basic solution containing free SN-38 carboxylate has a concentration of 2-25 mg/ml. In a particular embodiment, the basic solution containing free SN-38 carboxylate has a concentration of 2-12 mg/ml.

In a particular embodiment, the micelles according to the invention are those where each individual micelle in the micellar aqueous dispersion comprises only one type of therapeutic agent useful for the treatment of cancer. In another particular embodiment, the micellar aqueous dispersion is that where the free therapeutic agent is SN-38 lactone. In another particular embodiment, the micellar aqueous dispersion is that where each individual micelle comprises two or more therapeutic agents useful for the treatment of cancer.

The peptide conjugate of SN-38 of formula (I) can be generated wholly or partly by chemical synthesis. The amino acids required for the preparation of compounds of formula (I) are commercially available. The compounds of formula (I) can be prepared easily, for example by synthesis in liquid-phase or, preferably, by solid-phase peptide synthesis, for which there are a number of procedures published (see M. Amblard, et al., “Methods and protocols of modern solid-phase peptide synthesis. Molecular Biotechnology 2006, Vol. 33, p. 239-254). The compounds of formula (I) can also be prepared by any combination of liquid-phase synthesis and/or solid-phase synthesis. For example, by synthesizing the body of the peptide P through solid-phase synthesis and, subsequently removing protecting groups in solution. The binding of SN-38 to the linker L and peptide P can be performed in solid-phase or in solution. The construction of the linker L can also be prepared by any combination of liquid-phase synthesis and/or solid-phase synthesis.

The peptides of the invention can also be obtained by generation of a DNA template and subcloning into an expression vector (see J. H. Lee et al. Eur. J. 30 Biochem. 2001. Vol. 268. pp. 2004-2012).

Compound of formula (Ia) can be prepared by a process comprising reacting a compound of formula (III) with a compound of formula (IV) as described above to yield a compound of formula (Ia).

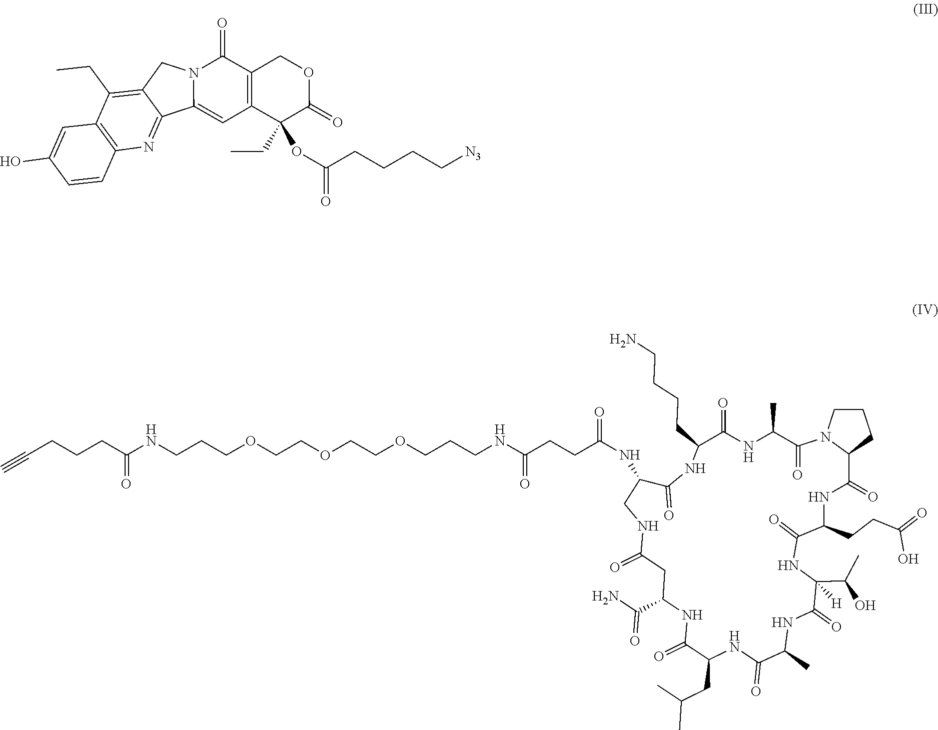 [SMILES: CCc1c2c(nc3ccc(O)cc13)-c1cc3c(c(=O)n1C2)COC(=O)[C@@]3(CC)OC(=O)CCCCN=[N+]=[N-] ; C#CCCCC(=O)NCCCOCCOCCOCCCNC(=O)CCC(=O)N[C@H]1CNC(=O)C[C@@H](C(N)=O)NC(=O)[C@H](CC(C)C)NC(=O)[C@H](C)NC(=O)[C@H]([C@@H](C)O)NC(=O)[C@H](CCC(=O)O)NC(=O)[C@@H]2CCCN2C(=O)[C@H](C)NC(=O)[C@H](CCCCN)NC1=O]

The preparation of pharmaceutically acceptable salts of the compounds of formula (I) can be carried out by methods known in the art. For instance, they can be prepared from the parent compound, which contains a basic or acidic moiety, by conventional chemical methods. Generally, such salts are, for example, prepared by reacting the free acid or base forms of these compounds with a stoichiometric amount of the appropriate pharmaceutically acceptable base or acid in water or in an organic solvent or in a mixture of them.

The process for preparing the micelles from these peptide conjugates of SN-38 and SN-38 lactone by their dissolution in water at pH<7 by spontaneous micellization, in particular pH<3 is also part of the invention. Such micelle acidic solutions can neutralize a basic solution containing free SN-38 carboxylate at concentrations of up to 25 mg/ml. Upon acidification of the basic pH, the appearance of free SN-38 lactone due to the pH change leads to inter-molecular interactions between free SN-38 lactone molecules and the SN-38 molecule conjugated in the peptide conjugate of SN-38. Generally, the micelles may be formed with a gentle mixing. Generally, they are formed in less than 30 min. Generally, the micelles are formed at room temperature.

The system produces drug-drug cocrystals at the nanometer size scale, comprised of two forms of SN-38 lactone, a free form and a conjugated form. Under such conditions, free SN-38 lactone does not form microcrystals or larger solid structures and remains apparently soluble at concentrations up to 4 mg/ml.

Such micellar aqueous dispersions of peptide conjugates of SN-38 containing free SN-38 lactone can be easily filtered through 0.22 and/or 0.45 μm pore filters without any loss of free SN-38 lactone. For instance, the filtration may be carried out with syringe 0.45 or 0.22 polypropylene filters.

The micellar aqueous dispersion of the present invention can also be freeze dried. Upon reconstitution with water, it conserves the same amount of soluble SN-38 lactone. The lyophilization may be carried out for example with a Telstar freeze-drier.

In a particular embodiment, the concentration of the peptide conjugate of the micelle in the micellar aqueous dispersion is up to 50 mg/ml. In another particular embodiment, the concentration of the peptide conjugate of the micelle in the micellar aqueous dispersion is from 1 to 50 mg/ml. In another particular embodiment, the concentration of the peptide conjugate of the micelle in the micellar aqueous dispersion is from 1 to 20 mg/ml. In another particular embodiment, the concentration of the peptide in the micellar aqueous dispersion is from 5 to 20 mg/ml. In another particular embodiment, the concentration of the peptide conjugate in the micellar aqueous dispersion is from 10 to 20 mg/ml.

Generally, the concentration of the peptide conjugate of the micelles of the present invention in the micellar aqueous dispersion of is selected from 20, 10, and 5 mg/mL.

In another particular embodiment, the proportion of free SN-38 lactone in the micellar aqueous dispersion is up to 25 mg/mL. In another particular embodiment, the proportion of free SN-38 lactone in the micellar aqueous dispersion is from 2-25 mg/ml. In another particular embodiment, the proportion of free SN-38 lactone in the micellar aqueous dispersion is from 2-12 mg/ml. In another particular embodiment, the proportion of free SN-38 lactone ranges from 1-8 mg/ml. in particular 8, 4, 2, 1 mg/mL.

In a particular embodiment, the micelle of the present invention is selected from the group consisting of: a) a micelle of a peptide conjugate of formula (Ia) in which the inner core is loaded with SN-38 wherein the concentration of th peptide conjugate of SN-38 in the micellar aqueous dispersion is 20 mg/ml and the concentration of SN-38 is 16 mg/ml;
b) a micelle of a peptide conjugate of formula (Ia) in which the inner core is loaded with SN-38, wherein the concentration of th peptide conjugate of SN-38 in the micellar aqueous dispersion is 20 mg/ml and the concentration of SN-38 is 9 mg/ml; c) a micelle of a peptide conjugate of formula (Ia) in which the inner core is loaded with SN-38 wherein the concentration of th peptide conjugate of SN-38 in the micellar aqueous dispersion is 10 mg/ml and the concentration of SN-38 is 9 mg/ml; and d) a micelle of a peptide conjugate of formula (Ic) in which the inner core is loaded with SN-38 wherein the concentration of th peptide conjugate of SN-38 in the micellar aqueous dispersion is 20 mg/ml and the concentration of SN-38 is 9 mg/ml.

In another particular embodiment, the micelle of the present invention is e) a micelle of a peptide conjugate of formula (Ia) in which the inner core is loaded with camptothecin lactone wherein the concentration of the peptide conjugate of SN-38 in the micellar aqueous dispersion is 20 mg/ml and the concentration of camptothecin lactone is 0.25-4 mg/ml, in particular 0.25-1 mg/ml. In another particular embodiment, the micelle of the present invention is f) a micelle of a peptide conjugate of formula (Ia) in which the inner core is loaded with camptothecin lactone and SN-38, wherein the concentration of the peptide conjugate of SN-38 in the micellar aqueous dispersion is 20 mg/ml and the concentration of the camptothecin lactone is 0.5-1 mg/ml and the concentration of the SN-38 is 0.5-1 mg/ml.

It is also part of the invention the pharmaceutical composition comprising a therapeutically effective amount of micelles as defined above, together with appropriate amounts of pharmaceutically acceptable carriers or excipients.

The term “therapeutically effective amount” as used herein, refers to the amount of a compound (micelles according to the invention) that, when administered, is enough to prevent development of, or alleviate to some extent, one or more symptoms of the disease which is addressed. The particular dose of compound administered according to this invention will of course be determined by the particular circumstances surrounding the case, including the compound administered, the via of administration, the particular condition being treated, and similar considerations.

The term “pharmaceutical composition” refers to a mixture of a compound described herein with other chemical components, such as diluents or carriers. The pharmaceutical composition facilitates administration of the compound to an organism. The terms “pharmaceutically acceptable excipients or carriers” refers to pharmaceutically acceptable material, composition or vehicle. Each component must be pharmaceutically acceptable in the sense of being compatible with the other ingredients of the pharmaceutical composition. It must be also suitable for use in contact with tissues or organs of humans and animals without excessive toxicity, irritation, allergic response, immunogenicity, or other problems or complications commensurate with a benefit/risk ratio.

The compositions of the present invention may be administered in parenteral form suitable for injection such as intravenous bolus injections, intravenous infusion, implantation into the body, oral, intratecal, or intranasal.

An important feature of the micelles of the present invention is their bioactivity inhibiting cell growth of the tested tumor cell lines. As it is illustrated in the Examples, the compounds of the present invention show antitumoral properties in several cancer cell lines. Cancer is a heterogeneous disease characterized by the accumulation of tumor cells, which can cause the death of both animals and humans. It is also one of the leading cause of death from disease among children and adolescents. Although substantial progress has been made in the treatment of several types of cancer over the past five decades, in particular, childhood cancer, progress against other types has been limited.

The annual incidence of tumors in children is between 100-160 cases per million. There is an annual risk of 1 in 500 children under 15. This incidence is slightly lower in industrialized countries. Brain and spinal cord tumors account for 25% of all neoplasms (40-50% of all pediatric solid tumors). Despite progress, survival data for these patients remains low, at around 55%. This contrasts with the substantial survival gains in recent years seen in other types of patients, such as those affected by leukaemia or extracranial tumours.

Conventional methods of treating cancer include surgical treatments, the administration of chemotherapeutic agents, and recently immune response-based therapy which involve the administration of an antibody or antibody fragment which may be conjugated to a therapeutic moiety. However, to date, such treatments have been of limited success.

Camptothecin, one of the four major structural classifications of plant-derived anti-cancerous compounds, is a cytotoxic alkaloid which consists of a pentacyclic ring structure containing a pyrrole (3, 4 β) quinoline moiety, an S-configured lactone form, and a carboxylate form. Irinotecan is made from natural camptothecin which is found in the Chinese ornamental tree Camptotheca acuminata. Several types of cancers have been treated at some point with irinotecan, among them glioblastoma multiforme (GBM) in the case of adults, and diffuse intrinsic pontine glioma (DIPG), paediatric glioblastoma (pGBM), neuroblastoma, rhabdomyosarcoma, Ewing sarcoma, and retinoblastoma in the case of children.

Gliomas are a group of tumors that start in the glial cells of the brain or the spine, comprise about 30 percent of all brain tumors and central nervous system tumours, and 80 percent of all malignant brain tumours. Often, treatment for brain gliomas is a combined approach, using surgery, radiation therapy, and chemotherapy. DIPG primarily affects children, usually between the ages of 5 and 7. Unfortunately, this is the majority of brain stem tumors, comprising 60-70%, and it is the one with the worst prognosis of all.

No treatment has been shown to be effective and the average survival is 9 months. Radiation and the administration of steroids is the only thing that has a palliative effect and that increases survival very discreetly. So far, there is no useful chemotherapy and multiple clinical trials have been tested, without favourable results. Pediatric glioblastomas are another group of tumors comprises one third of hemispheric tumors. It has a peak incidence at 8 and 12 years. Glioblastomas also affects adult patients, aroung 2-3 cases per 100.000 population.

Ewing Sarcoma is the second cause of malignant bone tumor in children and adolescents. The annual incidence is 0.6 per million inhabitants. It is rare before the age of 5 and the peak incidence is between 10 and 15 years, affecting more boys than girls, but this relationship in the sex varies according to the age range. The most common places for it to start are the pelvic (hip) bones, the chest wall (such as the ribs or shoulder blades), or in the middle of the leg bones. Ewing sarcoma can also present as an extra-skeletal lesion in the absence of bone injury. In this variant, there is a high risk of lymphatic spread and treatment is usually similar to rhabdomyosarcoma.

Soft tissue sarcomas are divided into rhabdomyosarcoma and non-rhabdomyosarcoma. Rhabdomyosarcoma accounts for 50% of all soft tissue sarcomas in children. It is the third solid extracranial tumor in frequency, after neuroblastoma and Wilms' tumor. The age peak is bimodal, with a first peak between 2 and 5 years, and a second peak in adolescence, between 15 and 19 years. While sarcomas in adults occur mostly in the extremities, in children they can originate in any location in the body, both in skeletal muscle and soft tissue. The most affected area in children is the head and neck and the urogenital tract. The extremities are affected in 20% of patients. Overall survival is poor, except if the tumor is found in locations where it can be completely resected, which sometimes requires limb amputation or voiding. Survival varies from 7-70%, depending on the location.

Neuroblastoma is the most common extracranial solid tumor of the childhood. Due to its embryonic origin, neuroblastomas can be virtually found in any part of the sympathetic nervous system, but the most common localization of the locoregional disease is on the adrenal gland (44%). Children between 1.5-6 years of age at diagnosis still can still be cured with conventional treatments, but their probability of survival decreases when they are diagnosed with metastatic disease (stage 4 neuroblastoma). About 50% of the newly diagnosed patients already present metastasis to bone (60%), bone marrow (50%), lymph nodes (42%) and/or liver (15%) and need intensive chemotherapy treatment, surgery and radiotherapy, but their survival remains poor, and few advances have been made over the last decades.

Finally, retinoblastoma is the most common cause of eye tumor in children, with a global incidence of 1 in 20,000 live births. It typically occurs in the first 2 years of life. Of the 30-40% are bilateral, in these cases there is always a positive family history. Of the unilaterals, 10% have a germline mutation of the Rb gene, located on chromosome 13. If detected early, they have a 95% survival rate. In certain parts of the world, when the diagnosis is late, the survival is drastically reduced, to less than 20%. Treatment depends on the tumor control achieved. When tumor regression is not controlled by chemotherapy and brachytherapy, enucleation is recommended. Even if enucleation is performed, in some cases there is tumor invasion into the optic nerve, which will force extra chemotherapy treatment.

It is part of the invention the micelles as defined above, for use as a medicament. It is also part of the invention, the micelles as defined above, for use in the treatment of cancer in a mammal, including a human as they are active in all the types of cancer where have been tested. This aspect can also be formulated as the use of micelles as defined above for the preparation of a medicament for the treatment and/or prevention of a cancer in a mammal, including a human. The invention also relates to a method of treatment of a mammal, including a human, suffering from or being susceptible of suffering from cancer, said method comprising the administration to said patient of a therapeutically effective amount of micelles as defined above, together with pharmaceutically acceptable excipients or carriers.

In a particular embodiment, the micelles are for use as defined above, where the cancer is located in the brain. In another particular embodiment, the micelles or the aqueous micellar dispersion are for use as defined above, where the micelles or the aqueous micellar dispersions cross the blood brain barrier and release the free therapeutic agent in the brain parenchyma and the cerebrospinal fluid. In a particular embodiment, the micelles of the present invention are for use as defined above, where the cancer treatment comprises the treatment of a tumor selected from the group consisting of extracranial solid tumors, eye tumors and CNS tumors. In another particular embodiment, either the micelles of the present invention are for use as defined above, where the cancer is selected from the group consisting of adult glioma, pediatric gliomas, retinoblastoma, Ewing sarcoma, DIPG, neuroblastoma, medulloblastoma, ependymoma, atypical teratoid-rhabdoid tumors (ATRT) and rhabdomyosarcoma. In another particular embodiment, the micelles of the present invention are for use as defined above, where the cancer is a pediatric brain tumour. In another particular embodiment, the micelles are for use as defined above, where the pediatric gliomas are selected from the group consisting of diffuse intrinsic pontine glioma (DIPG), and pediatric high-grade glioma. In another particular embodiment, the micelles are for use as defined above, where the cancer is diffuse intrinsic pontine glioma. In another particular embodiment, the micelles are for use as defined above, where the cancer is pediatric high-grade glioma. In another particular embodiment, the micelles are for use as defined above, where the cancer is retinoblastoma. In another particular embodiment, the micelles are for use as defined above, where the cancer is Ewing Sarcoma. In another particular embodiment, the micelle is for use as defined above, where the cancer is neuroblastoma. In another particular embodiment, the micelles are for use as defined above, where the cancer is rhabdomyosarcoma.

In another particular embodiment the micelles are for use as defined above, where the cancer is medulloblastoma. In another particular embodiment, the micelles are for use as defined above, where the cancer is ependymoma. In another particular embodiment, the micelles are for use as defined above, where the cancer is an atypical teratoid-rhabdoid tumors (ATRT). In another particular embodiment, the micelle is for use as defined above, where the cancer is adult glioma. In another particular embodiment, the micelles are for use as defined above, where they are active against cancer cell lines and patient-derived xenografts of Ewing sarcoma, DIPG, pHGG, glioblastoma multiforme, rhabdomyosarcoma, retinoblastoma, medulloblastoma, ependymoma, atypical teratoid-rhabdoid tumors (ATRT) and neuroblastoma.

In another particular embodiment, the micelles are for use as defined above, where they are active against cancer cell lines used in these experiments included cancer types of pediatric gliomas including diffuse intrinsic pontine glioma (HSJD-DIPG-007), pediatric diffuse midline glioma (HSJD-DMG-001) and pediatric high-grade glioma (HSJD-GBM-001), and cells lines of other pediatric solid tumors such as Ewing sarcoma (A673), and rhabdomyosarcoma (RH4).

The micelles of the present invention can be used in the same manner as other known chemotherapeutic agents, i.e., in combination with other treatments, either simultaneously or sequentially, depending on the condition to be treated. They may be used alone or in combination with other suitable bioactive compounds. Thus, the micelles of the present invention are for use in the treatment of cancer in a mammal, including a human in combination therapy with a chemotherapeutic agent.

In a particular embodiment, the dose of free SN-38 is at least 1 mg/kg and as much as 12 mg/kg (preclinical studies in mice). In a particular embodiment, the micelles are to be administered in combination with another chemotherapeutic agent. In another particular embodiment, the micelles are administered simultaneously with another chemotherapeutic agent. In another particular embodiment, the micelles are administered separately, in any order, within a therapeutically effective interval.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Reference signs related to drawings and placed in parentheses in a claim, are solely for attempting to increase the intelligibility of the claim, and shall not be construed as limiting the scope of the claim. Furthermore, the present invention covers all possible combinations of particular and preferred embodiments described herein.

### EXAMPLES

Protected amino acids, handles and resins were supplied by: Luxembourg Industries (Tel-Aviv, Israel), Neosystem (Strasbourg, France), CalbiochemNovabiochem AG (Laufelfingen, Switzerland), Bachem AG (Bubendorf, Switzerland) or Iris Biotech (Marktredwitz, Germany). Other reagents and solvents used are summarized in Table 1.

TABLE 1
Commercials suppliers and reagents used. DCM passed through
an Al₂O₃ column. DMF is stored on molecular sieves 4Å
and nitrogen is bubbled in order to eliminate volatile agents.

| Commercial | |
|---|---|
| supplier | Reagents and solvents |
| Albatros Chem. Inc. | N-Hydroxybenzotriazole (HOBt) |
| Aldrich | Piperidine, piperazine, pyridine, 3-(4,5-dimethyl-2- |
| | thiazolyl)-2,5-diphenyl-2H-tetrazolium bromide (MTT), |
| | 3-(4,5-dimethylthiazol-2-yl)-5-(3-carboxymethoxyphenyl)- |
| | 2-(4-sulfophenyl)-2H-tetrazolium (MTS), |
| | tetrakis(triphenylphosphine)palladium(0), phenyl silane, |
| | sodium diethyldithiocarbamate, |
| | triisopropylsilane (TIS), 1,2-ethanedithiol (EDT) |
| | diisopropylcarbodiimide (DIC), |
| | diisopropylethylamine (DIEA), O-(benzotriazol-1-yl)- |
| | N,N,N′,N′-tetramethyluronium tetrafluoroborate, |
| | tris(3-hydroxypropyltriazolylmethyl)amine (THPTA), |
| | NaHCO₃, brine, |
| | N-(3-dimethylaminopropyl)-N′-ethylcarbodiimide |
| | hydrochloride, mono-tert-butyl succinate, DBU, |
| | sodium ascorbate, 5-hexynoic acid, |
| | Fmoc-Asp(OAI)-OH, Fmoc-Dap(Alloc)- OH, |
| | HCl, MgSO₄ |
| Applied GL Biochem | 1-Hydroxy-7-azabenzotriazole (HOAt) |
| Shangai | |
| CEM | Rink amide Protide resin, Cl-TCP(Cl) ProTide resin |
| Envigo | Athymic nude mice |
| Carbosynth | SN-38 |
| IRIS Biotech | All aminoacids except otherwise specified, 5-azido- |
| | pentanoic acid (N₃-pen-OH), Fmoc-TTDS-OH, Di-tert- |
| | butyl dicarbonate, |
| Kalichem | Trifluoro acetic acid (TFA) |
| Merck | Copper (II) sulphate (CuSO₄) |
| Novabiochem | Benzotriazol-1-yl-oxytripyrrolidinophosphonium |
| | hexaluorophosphate (PyBOP), Oxyma pure |
| Scharlau | Dichloromethane (DCM), dimethylformamide, (DMF), |
| | methanol (MeOH), acetonitrile (AcCN), DMSO, |
| | tbutylmethyl ether (tBuMeO), NMP |
| SDS | Acetone, toluene, dioxane |
| PCAS Biomatrix Inc. | ChemMatrix ® resin |

### General Methods for the Preparation of the Peptide Coniugiates of SN-38:

General considerations about the manual synthesis: Solid-phase peptide elongation and other solid-phase manipulations were carried out manually in polypropylene syringes fitted with a polyethylene porous disk. Solvents and soluble reagents were removed by suction. Washings between different synthetical steps were carried out with dimethylformamide (DMF) (5×30 s) and dichloromethane (DCM) (5×30 s) using 10 mL of solvent/g of resin each time.

General considerations about the microwave assisted synthesis: Microwave assisted solid-phase peptide synthesis was carried out on a Liberty Blue Automated Microwave Peptide Synthesizer using H-Rink amide Protide resin (loading: 0.56 mmols/g). Linear peptide was synthesized on a 0.5 mmol scale using a 5 excess of Fmoc-aminoacid (0.2M) relative to the resin.

Identification tests: The test used for the identification and control of the synthesis was the following: A) Kaiser colorimetric assay for the detection of solid-phase bound primary amines (E. Kaiser et al., Anal. Biochem. 1970, vol. 34, pp. 595-598); B) p-nitro phenyl ester test for secondary amines bound to solid-phase (A. Madder et al., Eur. J. Org. Chem. 1999, pp. 2787-2791).

Protocols used during the manual synthesis of the compounds: The compounds were synthesized at a 100 mol scale using the following methods and protocols: The resin for the manual synthesis was selected depending on the group Y: If Y is a OH, the terminus will be COOH, 2-chlorotrytil chloride resin will be choosen among others available. If Y is a NH₂, the terminus will be CONH₂, Rink amide MBHA resin resin will be chosen among others available.

Resin initial conditioning: The resin was conditioned by washing with MeOH (5×30 s), DMF (5×30 s), DCM (5×30 s), 1% TFA in DCM (1×30 s and 2×10 min), DCM (5×30 s), DMF (5×30 s), DCM (5×30s), 5% DIEA in DCM (1×30 s, 2×10 min), DCM (5×30 s), DMF (5×30 s).

Fmoc group removal: Removal of the 9-fluorenylmethyloxycarbonyl (Fmoc) protecting group was done with 20% (v/v) piperidine in DMF using a treatment of 30 s followed by two treatments of 10 minutes each. Two additional treatments with DBU, toluene, piperidine, DMF (5%, 5%, 20%, 70%) (2×5 min) were performed to ensure the removal of the Fmoc group from secondary amines (proline).

### Coupling Methods Described for 100 Pmol Scale:

Coupling method 1: The protected amino acid (4 eq., 400 μmols), TBTU (4 eq., 400 μmols, 128 mg) dissolved in DMF (1-3 mL/g resin) were added sequentially to the resin, subsequently DIEA was added (8 eq, 800 μmols, 136 μl). The mixture was allowed to react with intermittent manual stirring for 1 h. The solvent was removed by suction and the resin washed with DMF (5×30 s) and DCM (5×30 s). The extent of coupling was checked by the Kaiser colorimetric assay. The Fmoc group was removed with a 20% solution of piperidine in DMF (v/v) using a 30 s treatment and two treatments of 10 minutes. If the amino acid to be deprotected was a proline, an additional tretament with DBU, toluene, piperidine, DMF (5%, 5%, 20%, 70%) (2×5 min) was applied to ensure the removal of the Fmoc group.

Coupling method 2: The protected amino acid (4 eq. 400 μmols), PyBOP (4 eq., 400 μmols, 208 mg), HOAt (12 eq., 1.2 mmols, 163 mg) dissolved in DMF (1-3 mL/g resin) were added sequentially to the resin, subsequently DIEA was added (12 eq., 1.2 mmols, 204 μL). The mixture was allowed to react with intermittent manual stirring for 1 h. The solvent was removed by suction and the resin washed with DMF (5×30 s) and DCM (5×30 s). The coupling reaction was carried out twice under the same conditions. The extent of coupling was checked by the Kaiser colorimetric assay. The Fmoc group was removed with a 20% solution of piperidine in DMF (v/v) using a 30 s treatments and two treatments of 10 minutes. If the amino acid to be deprotected was a proline, an additional tretament with DBU, toluene, piperidine, DMF (5%, 5%, 20%, 70%) (2×5 min) was applied to ensure the removal of the Fmoc group.

Coupling method 3: The protected amino acid (4 eq., 400 μmols), PyBOP (4 eq., 400 μmols, 208 mg), HOBt (12 eq., 1.2 mmols, 162 mg) dissolved in DMF (1-3 mL/g resin) were added sequentially to the resin, subsequently DIEA was added (12 eq., 1.2 mmols, 204 μL). The mixture was allowed to react with intermittent manual stirring for 1 h. The solvent was removed by suction and the resin washed with DMF (5×30 s) and DCM (5×30 s). The coupling reaction was carried out twice under the same conditions. The extent of coupling was checked by the Kaiser colorimetric assay. The Fmoc group was removed with a 20% solution of piperidine in DMF (v/v) using a 30 s treatments and two treatments of 10 minutes. If the amino acid to be deprotected was a proline, an additional tretament with DBU, toluene, piperidine, DMF (5%, 5%, 20%, 70%) (2×5 min) was applied to ensure the removal of the Fmoc group.

Couplinq method 4, scale 100 μmols: The protected amino acid (3 eq., 300 μmols)), DIC (3 eq., 300 μmols, 46 μL) and Oxyma (3 eq., 300 μmols, 43 mg) in DCM/DMF (1:1). The mixture was allowed to react with intermittent manual stirring for 45 min. The solvent was removed by suction and the resin washed with DMF (5×30 s) and DCM (5×30 s). The extent of coupling was checked by the Kaiser colorimetric assay. The Fmoc group was removed with a 20% solution of piperidine in DMF (v/v) using a 30 s treatments and two treatments of 10 minutes. If the amino acid to be deprotected was a proline, an additional tretament with DBU, toluene, piperidine, DMF (5%, 5%, 20%, 70%) (2×5 min) was applied to ensure the removal of the Fmoc group.

Coupling method 5, scale 100 μmols: The protected amino acid (3 eq., 300 μmols)), DIC (3 eq., 300 μmols, 46 μL) and HOBt (3 eq., 300 μmols, 41 mg) in DCM/DMF (1:1). The mixture was allowed to react with intermittent manual stirring for 45 min. The solvent was removed by suction and the resin washed with DMF (5×30 s) and DCM (5×30 s). The extent of coupling was checked by the Kaiser colorimetric assay. The Fmoc group was removed with a 20% solution of piperidine in DMF (v/v) using a 30 s treatments and two treatments of 10 minutes. If the amino acid to be deprotected was a proline, an additional tretament with DBU, toluene, piperidine, DMF (5%, 5%, 20%, 70%) (2×5 min) was applied to ensure the removal of the Fmoc group.

Protocols used during the microwave assisted automated synthesis: The compounds were synthesized at a 500 μmol scale using the following methods and protocols: The resin for the microwave assisted automated synthesis was selected depending on the group Y: If Y is a OH, the terminus will be COOH, CI-TCP(CI) ProTide resin will be choosen among others available. If Y is a NHz, the terminus will be CONH₂, Rink amide ProTide resin resin will be chosen among others available.

Resin initial conditioninm: The resin was conditioned by washing with MeOH (5×30 m), DMF (5×30 s), DCM (5×30 s), 1% TFA in DCM (1×30 sand 2×10 min), DCM (5×30 s), DMF (5×30 s), DCM (5×30s), 5% DIEA in DCM (1×30 s, 2×10 min), DCM (5×30 s), DMF (5×30 s).

### Coupling and Deprotection Conditions for the Microwave Assisted Automated Peptide Synthesis:

### Coupling Conditions:

| CEM | | | Microwave | |
|---|---|---|---|---|
| preference | Reagents | Cycle | method | Exceptions |
| 1 | AA/DIC/Oxyma | Single | Standard | His: Use singe |
| | in DMF | coupling | | 50° C. 10 min |
| | | | | coupling |
| | | | | Arg: double coupling |

| Method | Ramp time | Total time | Max temp |
|---|---|---|---|
| Standard coupling | 20-30 s | 1:05 | 90° C. |
| 50° C. MW | N/A | 2:00 | Room Temp |
| | 30-75 s | 4.00 | 50° C. |
| Arg coupling | N/A | 1. 25:00 | Room Temp |
| | 2. 30-75s | 2. 2:00 | 75° C. |

| CEM | Deprotection | Microwave |
|---|---|---|
| preference | coktail | method |
| 1 | 10% (w/v) Piperazine in | Standard |
| | 10:90 (EtOH/NMP) | |
| 2 | 20% Piperidine (v/v) in | Standard |
| | DMF or NMP | |

| Method | Ramp time | Total time | Max Temp |
|---|---|---|---|
| Standard | 20-30 s | 1:05 | 90° C. |
| deprotection | | | |

### Methods for the Cyclization of the Peptide Sequence P:

Cyclization method 1: disulfide or diselenide bond: The cyclization was performed in solution after the cleavage from the resin or on resin after the selective deprotection of the Cys, Sec or Pen residues. The peptide was dissolved at a concentration of 100 μM in aqueous ammonium bicarbonate buffer 10 mM and pH 8.0. The solution was intensely stirred for 24 h at room temperature. After that, the product was acidified with TFA to pH 2-3, frozen and lyophilized.

Cyclization method 2: amide bond: The cyclization was performed on resin. The Fmoc group was removed with a 20% solution of piperidine in DMF (v/v) using a 30 s treatment and two treatments of 10 minutes. The N-terminal amine was protected with a Boc protecting group using Boc₂O (3 eq, 1000 μmol, 56 mg) and DIEA (30 eq, 3000 μmol, 240 μL). The OAI and Alloc groups were first deprotected by addition of tetrakis(triphenylphosphine)palladium(0) (0.1 eq, 10 μM, 12 mg), phenyl silane (10 eq, 1000 μmol, 123 mg) in DCM (3×15 min). The resin was washed with 0.02 M sodium diethylcarbamate in DCM (3×5 min). The coupling of the amino group of Dap and the carboxylate group of aspartic acid was then achieved by addition of PyBOP (4 eq, 400 μmols, 208 mg), HOAt (12 eq, 1.2 mmol, 163 mg), DMF (1-3 mL/g resin) and DIEA (12 eq, 1.2 mmol, 204 μL). The coupling was left 1.5 h and repeated overnight.

Cyclization method 3: amide bond: The cyclization was performed on resin. The Fmoc group was removed with a 20% solution of piperidine in DMF (v/v) using a 30 s treatment and two treatments of 10 minutes. The N-terminal amine was protected with a Boc protecting group using Boc₂O (3 eq, 1000 μmol, 56 mg) and DIEA (30 eq, 3000 μmol, 240 μL). The OAI and Alloc groups were first deprotected by addition of tetrakis(triphenylphosphine)palladium(0) (0.1 eq, 10 μM, 12 mg), phenyl silane (10 eq, 1000 μmol, 123 mg) in DCM (3×15 min). The resin was washed with 0.02 M sodium diethyldithiocarbamate in DCM (3×5 min). The coupling of the amino group of Dap and the carboxylate group of aspartic acid was then achieved by_2 cycles of 30 min of 4 equivalents of Oxyma (400 mols, 57 mg) and 4 of N,N′-Diisopropylcarbodiimide (DIC) (400 mols, 61 L).

Cyclization method 4: amide bond: The cyclization was performed on resin. The Fmoc group was removed with a 20% solution of piperidine in DMF (v/v) using a 30 s treatment and two treatments of 10 minutes. The N-terminal amine was protected with a Boc protecting group using Boc₂O (3 eq., 1000 μmols, 56 mg) and DIEA (30 eq., 3000 μmols, 240 μL). The OAI and Alloc groups were first deprotected by addition of tetrakis(triphenylphosphine)palladium(0) (0.1 eq., 10 μM, 12 mg), phenyl silane (10 eq., 1000 μmols, 123 mg) in DCM (3×15 min). The resin was washed with 0.02 M sodium diethyldithiocarbamate in DCM (3×5 min). The coupling of the amino group of Dap and the carboxylate group of aspartic acid was then achieved by_2 cycles of 1 hour of 4 equivalents of DIC (400 mols, 61 L) and 4 of HOBt (400 mols, 54 mg).

### General Methods for the Construction of the Linker L:

General method for the formation of disulfides: the disulfide bond can be accomplished by reaction of two thiols. The thiols are dissolved at a concentration of 100 μM in aqueous ammonium bicarbonate buffer 10 mM and pH 8.0 and the solution is intensely stirred for 24 h at room temperature. After that, the solution is acidified with TFA to pH 2-3, frozen and lyophilized.

General methods for the formation of thioethers: The thioether bond is accomplished by reaction of an N-terminal bromoacetyl group with a cysteine thiol as described in P. L. Barker et al. J. Med. Chem., 1992. vol 35, pp. 2040-2048.

General methods for the formation of ethers: Ether formation can be accomplished by reaction of an hydroxyl group with an halo alkyl compound, preferably under basic conditions as described in Greene's Protective Groups in Organic Synthesis, Fifth Edition. Peter G. M. Wuts. 2014 John Wiley & Sons, Inc. pp. 26-29.

General methods for the formation of esters: Ester formation can be accomplished by reaction of an hydroxyl group and a carboxylic acid using typical esterification conditions, such as Fischer esterification in the presence of acid catalysis, or alterntively with the reaction of the hydroxyl group with the corresponding acid chloride, as describede in Greene's Protective Groups in Organic Synthesis, Fifth Edition. Peter G. M. Wuts. 2014 John Wiley & Sons, Inc. pp. 271-279 General methods for the formation of thioesters: The thioester bond is accomplished by reaction of an thiol with a carboxilic acid as described in M. Kazemi et al., Journal of Sulfur Chemistry, 2015, vol. 36:6, pp. 613-623.

General methods for the formation of urethanes: The reaction of the hydroxyl group with an isocyanate can yield the corresponding urethane, as described here M. T. Nguyen et al., J. Org. Chem. 1998, 63, vol. 20, pp. 6878-6885.

General methods for the formation of silyl ethers: Reaction of an hydroxyl group with an halo trialkyl silyl yields the corresponding silyl ether. An acid scavening agent is usually needed, as described in Greene's Protective Groups in Organic Synthesis, Fifth Edition. Peter G. M. Wuts. 2014 John Wiley & Sons, Inc. pp. 456-463.

General methods for the formation of sulphonates: Reaction of an hydroxyl group with an alkyl or aryl sulphonyl halide yields the corresponding sulfphonate ester as described in F. David et al., Org. Process Res. Dev. 2010, 14, 4, pp. 999-1007.

General methods for the formation of phosphates: Reaction of an hydroxyl group with a dialkyl or diaryl phosphate ester having one hydroxy group under dehydrating condition or using Mitsunobu reaction conditions can form the corresponding phosphate ester where one of the substituents is the chain attached to the hydroxyl group.

General methods for the formation of ketals: Ketals can be formed by reaction of an hydroxyl group with an halomethylenoxy alkyl compound, or by addition of an hydroxyl group to a substituted dihydropyran or dihydrofuran under acidic conditions, as described in Greene's Protective Groups in Organic Synthesis, Fifth Edition. Peter G. M. Wuts. 2014 John Wiley & Sons, Inc. pp. 69-77.

General methods for the formation of hemiketals: The contact of an hydroxyl group with an aldehyde can yield the formation of the corresponding hemiketal, as described here https://www.cliffsnotes.com/study-guides/chemistry/organic-chemistry-ii/aldehydes-and-ketones/reactions-of-aldehydes-and-ketones.

General methods for the formation of carbamates: Carbamates can be formed by reaction of an hydroxyl group with an haloformate or an isocyanate, as described in Greene's Protective Groups in Organic Synthesis, Fifth Edition. Peter G. M. Wuts. 2014 John Wiley & Sons, Inc.. pp. 371-374.

General methods for the formation of carbonates: Carbonates can be formed by reaction of SN-38 with PNPC, as described in Eur J Pharm Biopharm, 2017, 115, 149-158 or triphosgene as described in J. Med. Chem. 2008, 51, 21,6916-6926.

Coupling of Fmoc-TTDS-OH: The coupling of the Fmoc-TTDS-OH (2 equivalents), was achieved by either 2 cycles of 30 min of 4 equivalents of oxyma and 4 of N,N′-Diisopropylcarbodiimide (DIC) in DMF or 4 equivalents of DIC and 4 of HOBt in DCM during 2 h. Followed by removal of the 9-fluorenylmethyloxycarbonyl (Fmoc) protecting group with 20% (v/v) piperidine in DMF using a treatment of 30 s followed by two treatments of 10 minutes each.

Coupling of 5-hexynoic acid: The coupling of the 5-hexynoic acid (2 eq., 200 mols, 23 mg), was achieved by either 2 cycles of 30 min of 4 equivalents of Oxyma (400 mols, 57 mg) and 4 of N,N′-diisopropylcarbodiimide (DIC) (400 mols, 61 L) in DMF:DCM (1:1) or 4 equivalents of DIC (400 mols, 61 L) and 4 of HOBt (400 mols, 54 mg) in DMF:DCM 1:1 in DCM during 4 h or 2 equivalents of PyBOP (400 mols, 208 mg) in DMF:DCM 1:1, 6 equivalents of HOAt (600 μmols, 81.5 mg) and 6 equivalents of DIEA (600 μmols, 102 L) for 1.5 h in DMF. The solvent was removed by suction, the resin washed with DMF (5×30 s) and DCM (5×30 s). The coupling was repeated under the same conditions. The extent of coupling was monitored using the Kaiser colorimetric assay.

Coupling diglycolic anhidride: The coupling of diglycolic anhidride (10 eq., 1000 mols, 116 mg), was achieved by_2 cycles of 60 min of 10 equivalents of DIEA (1000 mols, 174 L) in DMF. The solvent was removed by suction, the resin washed with DMF (5×30 s) and DCM (5×30 s). The coupling was repeated under the same conditions. The extent of coupling was monitored using the Kaiser colorimetric assay.

General methods for the cleavage from the resin: Final cleavage of the resin and side-chain deprotection: It was carried out by treating resin with TFA (95%), H₂O (2.5%) and TIS (2.5%) (2 h). Tert-butyl methyl ether was added to the obtained product and the mixture was centrifuged (3×8 min). The supernatant was discarded and the pellet was resuspended in a mixture of H₂O, MeCN and TFA (1000:1000:1). The product was filtered out and frozen.

General methods for the characterization of the compounds: The compounds were characterized by UPLC (Acquity high-class system (PDA detector, sample manager FNT and Quaternary solvent manager, Acquity BEH C18 (50×2 mm×1.7 μm) column, 0.61 mL/min and MeCN (0.036% TFA) and H₂O (0.045% TFA) were used as solvents. In all cases, 2-min linear gradients were used) and UPLC-MS spectrometry (Waters high class (PDA detector, sample manager FNT and Quaternary solvent manager) coupled to an electrospray ion source ESI-MS Micromass ZQ and using the MassLynx 4.1 software (Waters, Milford, MA). Using a BEH C18 column (50×2.1 mm×1.7 μm, Waters). The flow rate was 0.6 mL/min, and MeCN (0.07% formic acid) and H₂O (0.1% formic acid) were used as solvents. Samples were analysed with positive ionisation: the ion spay voltage was 30 V and the capillary temperature was 1 kV). The exact mass was obtained by Mass Spectrometer: LTQ-FT Ultra (Thermo Scientific) with sample introduction in Direct infusion (Automated Nanoelectrospray). The NanoMate (Advion BioSciences, Ithaca, NY, USA) aspirated the samples from a 384-well plate (protein Lobind) with disposable, conductive pipette tips, and infused the samples through the nanoESI Chip (which consists of 400 nozzles in a 20×20 array) towards the mass spectrometer. Spray voltage was 1.70 kV and delivery pressure was 0.50 psi; the ionization was NanoESI, positive ionization.

NMR experiments were carried out on a Bruker Avance Ill 600 MHz spectrometer equipped with a TCI cryoprobe. Samples were prepared by dissolving compounds in 90% H₂O/10% D₂O at 3-4 mM and pH was adjusted to 2-3. Chemical shifts were referenced to internal sodium-3-(trimethylsilyl)propanesulfonate (DSS). Suppression of the water signal was achieved by excitation sculpting. Residue specific assignments were obtained from 2D total correlated spectroscopy (TOCSY) and correlation spectroscopy (COSY) experiments, while 2D nuclear Overhauser effect spectroscopy (NOESY) permitted sequence specific assignments. 13C resonances were assigned from 2D 1H13C HSQC spectra. All experiments were performed at 298 K except NOESY spectra that were acquired at 278 K. Amide proton temperature coefficients were determined from a series of onedimensional spectra acquired between 278 and 308 K. The TOCSY and NOESY mixing times were 70 and 250 ms, respectively.

### Example 1: Preparation of (S)-tert-butyl (4,11-diethyl-4-hydroxy-3,14-dioxo-3,4,12,14-tetrahydro-1H-pyrano[3′,4′:6,7]indolizino[1,2-b]quinolin-9-yl) carbonate

To a 250 ml flask, 1.5 g of 7-ethyl-10-hydroxy-camptothecin (SN-38), 1.3 equivalents of di-tert-butyl dicarbonate (1.15 mL) and excess of dry pyridine (9 mL) were added in 150 ml of dry DCM. The mixture was stirred at room temperature overnight. Afterwards the reaction mixture was washed with HCl (0,5 N)×3, saturated NaHCO₃×1 and brine. The organic layer was dried with MgSO₄ and the solvents were removed under vacuum. No further purification was performed. Reverse phase UPLC-PDA: linear gradient from 0 to 100% MeCN in H₂O in 2 minutes using a Acquity BEH C18 (50×2 mm×1.7 μm) column, 0.61 mL/min and MeCN (0.036% TFA) and H₂O (0.045% TFA) were used as solvents; Retention time: 1.89 min. Yield: 96%, [M+H]ₑₓₚ⁺: 493.5 Da. 1H NMR (400 MHz, choroform-d) 6 7.81 (d, J=9.2 Hz, 1H), 7.45 (d, J=2.5 Hz, 1H), 7.25-7.20 (m, 1H), 6.91-6.85 (m, 1H), 5.32 (d, J=16.3, 1H), 4.86 (d, J=1 Hz, 6H), 3.85 (s, 1H), 2.73 (q, J=7.7 Hz, 2H), 1.60 (s, 1H), 1.57-1.41 (m, 3H), 1,21 (s, 10H), 0.99 (t, J=7.7 Hz, 3H), 0.61 (t, J=7.3 Hz, 3H).

### Example 2: Preparation of (S)-9-((tert-butoxycarbonyl)oxy)-4,11-diethyl-3,14-dioxo-3,4,12,14-tetrahvdro-1H-pyrano[3′,4′:6,7]indolizino[1,2-b]quinolin-4-yl 5-azidopentanoate

In a round bottom flask, 400 mg of (S)-tert-butyl (4,11-diethyl-4-hydroxy-3,14-dioxo-3,4,12,14-tetrahydro-1H-pyrano[3′,4′:6,7]indolizino[1,2-b]quinolin-9-yl) carbonate (Example 1), 1.6 equivalents of 5-azido-pentanoic acid (193 mg) were purged with N₂ before being dissolved in dry DCM. The mixture was cooled at 0° C. 1.4 equivalents of N-(3-Dimethylaminopropyl)-N′-ethylcarbodiimide hydrochloride (218 mg) was then added and the mixture was stirred at 0° C. for 1 hour and at r.t. Overnight. The mixture was washed with NaHCO₃ saturated ×3, HCl(0,1N)×2 and brine. Then organic layer was dried with MgSO₄ and removed under vacuum. The compound was used without further purification. [M+H]ₑₓₚ⁺: 618.36 Da.

### Example 3: Preparation of (S)-4,11-diethyl-9-hydroxy-3,14-dioxo-3,4,12,14-tetrahydro-1H-pyrano[3′,4′:6,7]indolizino[1,2-b]quinolin-4-yl 5-azidopentanoate (modification of SN-38 with an azide as a result of Examples 1-2)

In a round bottom flask, 1.5 g of (S)-9-((tert-butoxycarbonyl)oxy)-4,11-diethyl-3,14-dioxo-3,4,12,14-tetrahydro-1H-pyrano[3′,4′:6,7]indolizino[1,2-b]quinolin-4-yl 5-azidopentanoate (Example 2) was stirred in 50 ml HCl (4N in dioxane) during 2 hours at r.t. Afterwards, the solvent was removed under vacuum and the crude mixture was purified with with silica using (DCM/MeOH (10%)) with a purity higher than 95%. Total yield from example 1: 8%, [M+H]⁺: 518.58 Da. Reverse phase UPLC-PDA: linear gradient from 0 to 100% MeCN in H₂O in 2 min using a Acquity BEH C18 (50×2 mm×1.7 μm) column, 0.61 mL/min and MeCN (0.036% TFA) and H₂O (0.045% TFA) were used as solvents; Retention time: 1.819 min. [M+H]ₑₓₚ⁺: 518.58 Da. ¹H-NMR (400 MHz, chloroform-d) δ 1.00 (t, 3H), 1.38 (t, 3H), 1.65 (m, 2H), 1.69 (m, 2H), 2.20 (m, 2H), 2.56 (m, 2H), 3.13 (q, 3H), 3.27 (td, 2H), 5.20 (s, 2H), 5.39-5.72 (dd, 2H), 7.42 (d, 1H), 7.48 (s, 1H), 7.62 (dd, 1H), 8.45 (d, 1H).

Methods for the alkyne-azide cycloaddition: The alkyne-azide cycloaddition (Click reaction) coupling was performed in solution using the protocol described in S. F. M. van Dongen et al.; Bioconjugate Chem. 2009, vol. 20, pp. 20-23. This reaction was done without microwave following the procedure descrived in lumiprobe (https://www.lumiprobe.com/protocols/click-chemistry-dna-labeling) However, this reaction took around 2 days to be completed. The Cu was used with the ligant THTPA. As SN-38-N₃ was not soluble in H₂O, it was made the reaction only using DMF instead of buffer and DMSO because it is not soluble in water or even in the mixture (water/DMSO). Alternatively the alkyne-azide cycloaddition (Click reaction) of alkyne-TTDS-DapKAPETALD with SN-38-N₃ with was done using microwaves: To a microwave vial of 10 ml SN-38-N₃ (1.5 eq.), alkyne-TTDS-DapKAPETALD (1 eq.), CuTHTPA (0.15 eq.) and sodium ascorbate (0.3 eq.) were added and dissolved in 3 ml of DMF. The mixture was stirred from 2 to 4 hours with MW (discover SP MW) assisted at 30° C. during all reaction. The crude mixture was purified with HPLC semipreparative (C18).

Preparation of reagents for alkyne-azide cycloaddition (Click chemistry): 100 mM Copper (II)-THPTA Stock in 55% DMSO: 50 mg of copper (II) sulfate pentahydrate were dissolved in 1 mL of distilled water and 116 mg of tris(3-hydroxypropyltriazolylmethyl)amine (THPTA) ligand were dissolved in 1.1 mL of DMSO. Then, the two solutions were mixed. 5 mM Ascorbic Acid Stock: 18 mg of ascorbic acid were dissolved in 20 mL of distilled water.

General method for purification and characterization of products: The crude were purified by RP-HPLC at semi-preparative scale and characterized by UPLC (Acquity high-class system (PDA detector, sample manager FNT and Quaternary solvent manager, Acquity BEH C18 (50×2 mm×1.7 μm) column, 0.61 mL/min and MeCN (0.036% TFA) and H₂O (0.045% TFA) were used as solvents. In all cases, 2-min linear gradients were used) and UPLC-MS spectrometry (Waters high class (PDA detector, sample manager FNT and Quaternary solvent manager) coupled to an electrospray ion source ESI-MS Micromass ZQ and using the MassLynx 4.1 software (Waters, Milford, MA). Using a BEH C18 column (50×2.1 mm×1.7 μm, Waters). The flow rate was 0.6 mL/min, and MeCN (0.07% formic acid) and H₂O (0.1% formic acid) were used as solvents. Samples were analysed with positive ionisation: the ion spay voltage was 30 V and the capillary temperature was 1 kV). The exact mass was obtained by Mass Spectrometer: LTQ-FT Ultra (Thermo Scientific) with sample introduction in Direct infusion (Automated Nanoelectrospray). The NanoMate (Advion BioSciences, Ithaca, NY, USA) aspirated the samples from a 384-well plate (protein Lobind) with disposable, conductive pipette tips, and infused the samples through the nanoESI Chip (which consists of 400 nozzles in a 20×20 array) towards the mass spectrometer. Spray voltage was 1.70 kV and delivery pressure was 0.50 psi; the ionization was NanoESI, positive ionization. All peptides were obtained with a purity higher than 95%.

### Example 4: Preparation of Hexynoic-TTDS-Dap-Lys-Ala-Pro-Glu-Thr-Ala-Leu-Asp-NH₂ (hexvnoic-TTDS-SEQ ID NO: 7) with a amide bond between Dap side-chain amino group and Asp side-chain carboxilic acid

For the manual coupling of the first protected amino acid to the resin, the coupling method 4 was applied using Fmoc-Asp(OAI)-OH (118.5 mg). The subsequent amino acids were coupled sequentially as follows using coupling method 4:

| Add. | | | | | |
|---|---|---|---|---|---|
| order | Code | Amino acid | MW | m (mg) | eq |
| 1 | D | Fmoc-L-Asp(OAl)-OH | 395.4 | 118.5 | 3 |
| 2 | L | Fmoc-L-Leu-OH | 353.4 | 105.9 | 3 |
| 3 | A | Fmoc-Ala-OHH₂O | 329.3 | 98.7 | 3 |
| 4 | T | Fmoc-L-Thr(tBu)-OH | 397.5 | 119.1 | 3 |
| 5 | E | Fmoc-Glu(OtBu)- | 443.5 | 132.9 | 3 |
| | | OH H₂O | | | |
| 6 | P | Fmoc-L-Pro-OHH₂O | 355.4 | 106.5 | 3 |
| 7 | A | Fmoc-Ala-OHH₂O | 329.3 | 98.7 | 3 |
| 8 | K | Fmoc-Lys(Boc)-OH | 468.5 | 140.4 | 3 |
| 9 | Dap | Fmoc-L-Dap(Alloc)- | 410.4 | 123 | 3 |
| | | OH | | | |

Using 46 μL DIC and 43 mg Oxyma in DMF/DCM (1:1). The mixture was allowed to react with intermittent manual stirring for 45 min. After each coupling removal of the 9-fluorenylmethyloxycarbonyl (Fmoc) protecting group was done with 20% (v/v) piperidine in DMF using a treatment of 30 s followed by two treatments of 10 minutes each. Two additional treatments with DBU, toluene, piperidine, DMF (5%, 5%, 20%, 70%) (2×5 min) were performed to ensure the removal of the Fmoc group from secondary amines (proline). The cyclization was performed on resin following cyclization method 2: The Fmoc group was removed with a 20% solution of piperidine in DMF (v/v) using a 30 s treatment and two treatments of 10 minutes. The N-terminal amine was protected with a Boc protecting group using Boc₂O (3 eq., 1000 μmols, 56 mg) and DIEA (30 eq., 3000 μmols, 240 μL). The OAI and Alloc groups were first deprotected by addition of tetrakis(triphenylphosphine)palladium(0) (0.1 eq., 10 μM, 12 mg), phenyl silane (10 eq., 1000 μmols, 123 mg) in DCM (3×15 min). The resin was washed with 0.02 M sodium diethylcarbamate in DCM (3×5 min). The coupling of the amino group of Dap and the carboxylate group of aspartic acid was then achieved by addition of PyBOP (4 eq., 400 μmols, 208 mg), HOAt (12 eq., 1.2 mmols, 163 mg), DMF (1-3 mL/g resin) and DIEA (12 eq., 1.2 mmols, 204 μL). The coupling was left 1.5 h and repeated overnight.

Coupling of Fmoc-TTDS-OH: The coupling of the Fmoc-TTDS-OH (2 equivalents, 200 mols, 108.53 mg), was achieved by_4 equivalents of DIC (400 mols, 61 L) and 4 of HOBt (400 mols, 54 mg) in DCM during 2 h._Followed by removal of the 9-fluorenylmethyloxycarbonyl (Fmoc) protecting group with 20% (v/v) piperidine in DMF using a treatment of 30 s followed by two treatments of 10 minutes each.

Coupling of 5-hexynoic acid: For the coupling of the 5-hexynoic acid to the peptide anchored onto the resin the following protocol was used: hexynoic acid (4 eq., 400 μmols, 45 mg) in DMF (1-3 mL/g resin), PyBOP (4 eq., 400 μmols, 208 mg) and HOAt (12 eq., 1.2 mmols, 163 mg) were sequentially added to the resin followed by the addition of 12 eq. of DIEA (1.2 mmols, 204 L). The mixture was allowed to react with intermittent manual stirring for 1.5 h. The solvent was removed by suction, the resin washed with DMF (5×30 s) and DCM (5×30 s). The coupling was repeated under the same conditions. The extent of coupling was monitored using the Kaiser colorimetric assay.

The peptide was then cleaved and liophilized. Product characterization. Reverse phase UPLC: linear gradient from 20 to 60% MeCN in H₂O in 2 min using a Acquity BEH C18 (50×2 mm×1.7 μm) column, 0.61 mL/min and MeCN (0.036% TFA) and H₂O (0.045% TFA) were used as solvents; Retention time: 0.939 min. UPLC-MS [M+H]ₑₓₚ⁺: 1308.15 Da; Yield (synthesis and purification): 7.5%.

### Example 5: Preparation of Compound of Formula (Ia), G2B-001

Starting from Hexynoic-TTDS-DapKAPETALD prepared as in example 4, and using SN-38-N₃ prepared as in example 3, the following protocol was follow to achieve compound of formula (Ia): To a microwave vial of 10 ml 1.5 eq. of (S)-4,11-diethyl-9-hydroxy-3,14-dioxo-3,4,12,14-tetrahydro-1H-pyrano[3′,4′:6,7]indolizino[1,2-b]quinolin-4-yl 5-azidopentanoate, 1 eq. of Alkyne-TTDS-DapKAPETALD, 0.15 eq. of CuTHPTA and 0.3 eq. of sodium ascorbate were added and dissolved in 3 ml of DMF. The mixture was stirred from 2 to 4 hours with MW (CEM discover SP MW) assisted at 30° C. during all reaction. The crude was purified by RP-HPLC at semi-preparative scale. It was obtained the compound with a purity higher than 95%. Product characterization: Reverse phase UPLC-PDA: linear gradient from 0 to 100% MeCN in H₂O in 2 minutes using a Acquity BEH C18 (50×2 mm×1.7 μm) column, 0.61 mL/min and MeCN (0.036% TFA) and H₂O (0.045% TFA) were used as solvents; Retention time: 1.462 min. UPLC-MS [M+H]ₑₓₚ⁺: 1824.76 Da; Yield (synthesis and purification): 30%.

### Example 6: Preparation of Hexynoic-TTDS-Dap-Lys-Ala-Pro-Glu-Thr-Ala-Leu-Asp-NH₂ (Hexvnoic-TTDS-SEQ ID NO: 14) Lineal without an Amide Bond Between Dap Side-Chain Amino Group and Asp Side-Chain Carboxilic Acid. DapKAPETALD (SEQ ID NO:14), i.e., Linear Peptide

For the manual coupling of the first protected amino acid to the resin, the coupling method 4 was applied using Fmoc-Asp(OAI)-OH (118.5 mg). The subsequent amino acids were coupled sequentially as follows using coupling method 4:

| Add. | | | | | |
|---|---|---|---|---|---|
| order | Code | Amino acid | MW | m (mg) | eq |
| 1 | D | Fmoc-L-Asp(OAl)-OH | 395.4 | 118.5 | 3 |
| 2 | L | Fmoc-L-Leu-OH | 353.4 | 105.9 | 3 |
| 3 | A | Fmoc-Ala-OH H₂O | 329.3 | 98.7 | 3 |
| 4 | T | Fmoc-L-Thr(tBu)-OH | 397.5 | 119.1 | 3 |
| 5 | E | Fmoc-Glu(OtBu)- | 443.5 | 132.9 | 3 |
| | | OH H₂O | | | |
| 6 | P | Fmoc-L-Pro-OH H₂O | 355.4 | 106.5 | 3 |
| 7 | A | Fmoc-Ala-OH H₂O | 329.3 | 98.7 | 3 |
| 8 | K | Fmoc-Lys(Boc)-OH | 468.5 | 140.4 | 3 |
| 9 | Dap | Fmoc-L-Dap(Alloc)- | 410.4 | 123 | 3 |
| | | OH | | | |

Using 46 μL DIC and 43 mg Oxyma in DMF/DCM (1:1). The mixture was allowed to react with intermittent manual stirring for 45 min. After each coupling removal of the 9-fluorenylmethyloxycarbonyl (Fmoc) protecting group was done with 20% (v/v) piperidine in DMF using a treatment of 30 s followed by two treatments of 10 minutes each. Two additional treatments with DBU, toluene, piperidine, DMF (5%, 5%, 20%, 70%) (2×5 min) were performed to ensure the removal of the Fmoc group from secondary amines (proline).

Coupling of Fmoc-TTDS-OH: The coupling of the Fmoc-TTDS-OH (2 equivalents, 200 mols, 108.53 mg), was achieved by_4 equivalents of DIC (400 mols, 61 L) and 4 of HOBt (400 mols, 54 mg) in DCM during 2 h._Followed by removal of the 9-fluorenylmethyloxycarbonyl (Fmoc) protecting group with 20% (v/v) piperidine in DMF using a treatment of 30 s followed by two treatments of 10 minutes each.

Coupling of 5-hexynoic acid: For the coupling of the 5-hexynoic acid to the peptide anchored onto the resin the following protocol was used: hexynoic acid (4 eq., 400 μmols, 45 mg) in DMF (1-3 mL/g resin), PyBOP (4 eq., 400 μmols, 208 mg) and HOAt (12 eq., 1.2 mmols, 163 mg) were sequentially added to the resin followed by the addition of 12 eq. of DIEA (1.2 mmols, 204 L). The mixture was allowed to react with intermittent manual stirring for 1.5 h. The solvent was removed by suction, the resin washed with DMF (5×30 s) and DCM (5×30 s). The coupling was repeated under the same conditions. The extent of coupling was monitored using the Kaiser colorimetric assay.

The OAI and Alloc groups were deprotected by addition of tetrakis(triphenylphosphine)palladium(0) (0.1 eq., 10 μM, 12 mg), phenyl silane (10 eq., 1000 μmols, 123 mg) in DCM (3×15 min). The resin was washed with 0.02 M sodium diethylcarbamate in DCM (3×5 min). The peptide was then cleaved and liophilized.

Product characterization. Reverse phase HPLC: linear gradient from 10 to 60% MeCN in H₂O in 30 min using a Xbridge 25 cm 3.5 m column, 1 mL/min and MeCN (0.1% TFA) and H₂O (0.1% TFA) were used as solvents; Retention time: 11.526 min. Yield (synthesis and purification): 9%.

### Example 7: Preparation of Compound of Formula (Ib), G2B-001 Lineal without an Amide Bond Between Dap Side-Chain Amino Qroup and Asp Side-Chain Carboxilic Acid

Starting from Hexynoic-TTDS-DapKAPETALD lineal prepared as in example 6 and using SN-38-N₃ prepared as in example 3, the following protocol was follow to achieve compound of formula (Ib) also named G2B-001 lineal: To a microwave vial of 10 ml 1.5 eq. of (S)-4,11-diethyl-9-hydroxy-3,14-dioxo-3,4,12,14-tetrahydro-1H-pyrano[3′,4′:6,7]indolizino[1,2-b]quinolin-4-yl 5-azidopentanoate, 1 eq. ofAlkyne-TTDS-DapKAPETALD lineal, 0.15 eq. of CuTHPTA and 0.3 eq. of sodium ascorbate were added and dissolved in 3 ml of DMF. The mixture was stirred from 2 to 4 hours with MW (CEM discover SP MW) assisted at 30° C. during all reaction. The crude was purified by RP-H PLC at semi-preparative scale. It was obtained the compound with a purity higher than 95%. Product characterization: Reverse phase HPLC-MS: linear gradient from 0 to 80% MeCN in H₂O in 7 minutes using a Luna 3 μm C18 (2) 100A 50×2.1 mm column, 0.85 mL/min and MeCN (0.1% Formic acid) and H₂O (0.1% Formic acid) were used as solvents; Retention time: 5.08 min. UPLC-MS [M+H]ₑₓₚ⁺: 1845.10 Da; Yield (synthesis and purification): 22%.

### Example 8: Preparation of N₃-Pen-SN-38

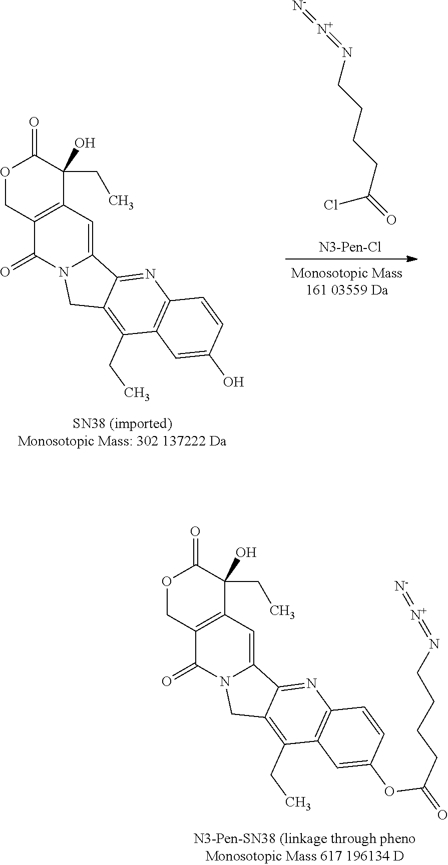 [SMILES: CCc1c2c(nc3ccc(O)cc13)-c1cc3c(c(=O)n1C2)COC(=O)[C@]3(O)CC ; [N-]=[N+]=NCCCCC(=O)Cl ; CCc1c2c(nc3ccc(OC(=O)CCCCN=[N+]=[N-])cc13)-c1cc3c(c(=O)n1C2)COC(=O)[C@]3(O)CC]

In a two neck round bottom flask, add 1 eq. of SN-38 (600 mg), and dissolve in 100 ml per gram dry DCM (60 ml). Turbid solution is observed. Cool this reaction mixture to 0-2° C. and add 6 eq. of DIEA (2 ml) and stir for 15 minutes. After 15 min, portion wise add 3 eq. of N₃-Pen-Cl (700 mg) [dissolved in 500 uL dry DCM]. Cool it for 15 mins and then continue stirring at room temperature. Reaction progress is checked by HPLC. When starting material is below 3% take the reaction for work up. Initial volume of reaction mixture ˜45 ml which is further made-up to 300 ml using dry DCM. Extract thrice with distilled water (20% volume of final volume of reaction mixture) 3×3 min×60 ml. Dry DCM layer using sodium sulphate (Na₂SO₄) for 30 mins and evaporate on rotary evaporator. Treat solid obtained with 80:20 Hex: Ether twice and keep for drying in desiccator. Yield after work up, 730 mg. Product characterization. Reverse phase HPLC: linear gradient from 10 to 90% MeCN in H₂O in 30 min using a SS column 250×4.6 mm packed with C-18 silica gel for chromatography R (5 μm), 1 mL/min and MeCN (0.1% TFA) and H₂O (0.1% TFA) were used as solvents; Retention time: 19.730 min. Mass observed=518.20 Da, Mass calculated=517.19 Da.

### Example 9: Preparation of Compound of Formula (Ic), G2B-003

Starting from Hexynoic-TTDS-DapKAPETALD prepared as in example 4, and using N₃-Pen-SN-38 prepared as in example 8, the following protocol was follow to achieve compound of formula (Ic) also named G2B-003. To a vial of 10 ml 1.5 eq. of N₃-Pen-SN-38, 1 eq. of Hexynoic-TTDS-DapKAPETALD, 0.15 eq. of CuTHPTA and 0.3 eq. of sodium ascorbate were added and dissolved 3 ml of DMF. The mixture was stirred from 2 to 4 hours with Microwave (CEM discover SP MW) assisted at 30° C. during all reaction. The crude was purified by RP-HPLC at semi-preparative scale. It was obtained the compound with a purity higher than 95%. Product characterization: Reverse phase HPLC: linear gradient from 20 to 70% MeCN in H₂O in 30 minutes using a using a Xbridge 25 cm 3.5 m column, 1 mL/min and MeCN (0.1% TFA) and H₂O (0.1% TFA) were used as solvents; Retention time: 12.00 min. UPLC-MS [M+H]ₑₓₚ⁺: 1826.15 Da; Yield (synthesis and purification): 17%.

### Example 10: Preparation of SN-38-O—CO—NH-Gly-COOH

A solution of glycine-COOBut (1 eq) and DMAP (2 eq) in dry DCM (15 mL) was added dropwise to a solution of bis(4-nitrophenyl) carbonate (1.3 eq.) in dry DCM (15 mL) and the resulting solution was stirred at 50° C. overnight. The reaction mixture was then diluted in DCM (150 mL) and washed with 0.5 N HCl (100 mL). The aqueous layer was washed with DCM (5×100 mL) and all the organic fractions were collected, dried over MgSO₄ and filtered. The solvent was evaporated under reduced pressure and the residue was purified by flash chromatography (Hexane:DCM:Et2O; 5:4:1). The resulting PNP-Gly-COOBut (1 eq) was reacted with SN-38 (1.2 eq) in the presence of DMAP (2 eq) in dry DCM. The reaction was was stirred at 50° C. overnight. The solvent was evaporated under reduced pressure and the residue was purified by flash chromatography. The t-butyl ester group was removed by treating SN-38-O—CO—NH-Gly-COOBut with TFA:DCM:TIS (40:40:20) during 6 h. Afterwards the solvent was evaporated. Toluene was added twice to remove the traces of TFA. No further purification was performed.

### Example 11: Preparation of Boc-Val-Cit-PAB-SN-38

In a round bottom flask, 1 eq of 7-ethyl-10-hydroxy-camptothecin (SN-38), 1 eq of Boc-Val-Cit-PAB-PNP (from iris biotech), 2 eq of DIEA and catalitic DMAP were stirred in dry DMF for 16 h at r.t. Afterwards, the reaction mixture was diluted with AcOEt and washed with HCl (0.5 M) (×3), and brine (×4). Then it was dried with MgSO₄, evaporated the volatiles the crude mixture was purified by silica cromatography (AcOEt:MeOH; 20:1; 15:1:9:1). The desired compound was obtained with a yield of 52% and purity higher than 90%. Yield 52%. Reverse phase UPLC-PDA: linear gradient from 0 to 100% MeCN in H₂O in 2 minutes using a Acquity BEH C18 (50×2 mm×1.7 μm) column, 0.61 mL/min and MeCN (0.036% TFA) and H₂O (0.045% TFA) were used as solvents; Retention time: 1.42 min. [M+H]ₑₓₚ⁺: 898.4 Da.

### Example 12: Preparation of H₂N-Val-Cit.PAB-SN-38

In a round bottom flask, 1 eq of Boc-Val-Cit-PAB-SN-38 prepared as in example 11 was stirred in a mixture of DCM:TFA:H₂O (44.75:49.75:0.5) during 5 minutes. Afterwards the solvent was evaporated. Toluene was added twice to remove the traces of TFA. No further purification was performed.

### Example 13: Preparation of G2B-004 (Id)

G2B-004 was prepared starting from diglycolic-DapKAPETALD (prepared using standard methods described above, coupling method 4) and 2 eq H₂N-Val-Cit-PAB-SN-38 (prepared as in example 12) using PyBOP (4 eq), HOAt (12 eq) dissolved in DMF (1-3 mL/g resin) followed by addition of DIEA (12 eq). The mixture was allowed to react with intermittent manual stirring for 1 h. The solvent was removed by suction and the resin washed with DMF (5×30 s) and DCM (5×30 s). The coupling reaction was carried out twice (1h and overnight). G2B-004 was then cleaved and liophilized. Product characterization. Reverse phase UPLC: linear gradient from 0 to 100% MeCN in H₂O in 2 min using a Acquity BEH C18 (50×2 mm×1.7 μm) column, 0.61 mL/min and MeCN (0.036% TFA) and H₂O (0.045% TFA) were used as solvents; Retention time: 1.17 min. UPLC-MS [M+H]ₑₓₚ⁺: 1807.84 Da. Yield (synthesis and purification): 21%.

### Example 14: Preparation of G2B-005 (Ie)

Nval-Pro-Gly-DapKAPETALID was prepared using standard methods described above (coupling method 4). G2B-005 was prepared starting from Nval-Pro-Gly-DapKAPETALD and 2 eq SN-38-O—CO—NH-Gly-OH prepared as in example 10 using PyBOP (4 eq), HOAt (12 eq) dissolved in DMF (1-3 mL/g resin) followed by addition of DIEA (12 eq). The mixture was allowed to react with intermittent manual stirring for 1 h. The solvent was removed by suction and the resin washed with DMF (5×30 s) and DCM (5×30 s). The coupling reaction was carried out twice (1h and overnight). G2B-005 was then cleaved and liophilized. Product characterization. Reverse phase UPLC: linear gradient from 0 to 100% MeCN in H₂O in 2 min using a Acquity BEH C18 (50×2 mm×1.7 μm) column, 0.61 mL/min and MeCN (0.036% TFA) and H₂O (0.045% TFA) were used as solvents; Retention time: 1.51 min. UPLC-MS [M+H]ₑₓₚ⁺: 1640.77 Da. Yield (synthesis and purification): 18%.

### Example 15: Preparation of Several Aqueous Micellar Dispersions Containing SN-38 Lactone Encapsulated within Micelles of SN-38 Peptide Coniugates

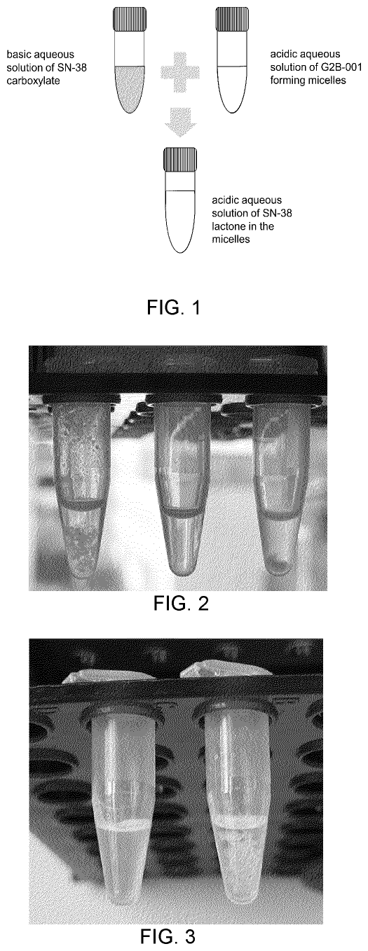

All formulations are simple mixtures of a basic aqueous solution (NaOH 0.05 N, in water) of free SN-38 carboxylate and an acidic aqueous solution (tartaric acid, 20 mg/mL (134 mM), in water) of G2B-001 or other SN-38 peptide conjugates (that form micelles) in which the concentrations and volumes of both components in basic and acid solutions can be modified. The result of mixing equal volumes of basic and acidic solutions is an acidic solution of pH<7 (experimental value is 2.3-2.5), completely translucent and physically stable over time (no precipitates or turbidity appear after weeks of storage at room temperature), containing SN-38 lactone encapsulated in G2B-001 micelles. A scheme of this process is shown in FIG. 1

### Example 15a: Preparation of G2B-001 Micelles at Concentration 20 mg/mL and Containing 16% w/w Free SN-38 Lactone. This Formulation is Known as G2B-002-20-16

In this formulation, the percentage weight/weight (% w/w) of free SN-38 in the final formulation is 16% w/w, i.e., free SN-38/(SN-38+G2B-001)*100=16%. Final concentrations in the aqueous formulation are 4 mg/mL for free SN-38 and 20 mg/mL for G2B-001 and the final pH is <3, ensuring that free SN-38 is in mostly as active lactone.

The particle size is 310 nm (Z-average) and the polydispersity index (PDI) is 0.55 (measured by dynamic light scattering).

### Example 15b: Preparation of G2B-001 Micelles at Concentration 20 mq/mL and Containing 9% w/w Free SN-38 Lactone. This Formulation is Known as G2B-002-20-9

In this formulation, the percentage weight/weight (% w/w) of free SN-38 in the final formulation is 9% w/w, i.e., free SN-38/(SN-38+G2B-001)*100=9%. Final concentrations in the aqueous formulation are 2 mg/mL for free SN-38 and 20 mg/mL for G2B-001 and the final pH is <3, ensuring that free SN-38 is in mostly as active lactone.

The particle size is 103 nm (Z-average) and the polydispersity index (PDI) is 0.50 (measured by dynamic light scattering). The solution is transparent.

### Example 15c: Preparation of G2B-001 Micelles at Concentration 10 mg/mL and Containing 5% w/w Free SN-38 Lactone. This Formulation is Known as G2B-002-10-5

In this formulation, the percentage weight/weight (% w/w) of free SN-38 in the final formulation is 5% w/w, i.e., free SN-38/(SN-38+G2B-001)*100=5%. Final concentrations in the aqueous formulation are 0.5 mg/mL for free SN-38 and 10 mg/mL for G2B-001 and the final pH is <3, ensuring that free SN-38 is in mostly as active lactone. The particle size is 40 nm (Z-average) and the polydispersity index (PDI) is 0.40 (measured by dynamic light scattering).

The following table illustrates the amounts of SN38 in the peptide and the amount of free SN-38 lactone:

| | | | | | Proportion | Proportion |
|---|---|---|---|---|---|---|
| | | | | | w/w of free | w/w of SN-38 |
| | | | | | SN-38 | and G2B-001 in |
| | | | | | lactone and | the micellar |
| | | | Conc. | Conc. | G2B-001 in | aqueous |
| | Conc. | Conc. | SN-38 | SN-38 | the micellar | dispersion |
| | G2B-001 | G2B-001 | lactone | lactone | aqueous | (includes the |
| Product | (mg/mL) | (mM) | (mg/mL) | (mM) | dispersion | conjugated SN-38) |
| G2B-002- | 20 | 11.0 | 4 | 10.2 | 16.7 | 34.6 |
| 20-16 | | | | | | |
| G2B-002- | 20 | 11.0 | 2 | 5.10 | 9.1 | 28.6 |
| 20-9 | | | | | | |
| G2B-002- | 10 | 5.5 | 1 | 2.55 | 9.1 | 28.6 |
| 10-5 | | | | | | |

### Example 15d: Preparation of G2B-003 Micelles at Concentration 20 mg/mL and Containing 5% w/w Free SN-38 Lactone. This Formulation is Known as G2B-006-20-9

It was prepared as G2B-002-20-9 but with the peptide conjugate of formula (Ic) instead of the peptide conjugate of formula (Ia). In this formulation, the percentage weight/weight (% w/w) of free SN-38 in the final formulation is 9% w/w, i.e., free SN-38/(SN-38+G2B-003)*100=9%.

### Example 16: Load of Soluble SN-38 Lactone in the Micellar Systems

To study the loading efficiency of soluble SN-38 lactone in the invented systems, G2B-001 or G2B-003 micelles at concentration 40 mg/mL in acid solution (0.5 mL) were added to SN-38 carboxylate at concentration 4 or 8 mg/mL in basic solution (0.5 mL) in plastic vials (1.5 mL). After gentle mixing, the samples were kept at room temperature (18-22° C.) for 0.5 h. Then, the resulting solutions were filtered through 0.45 μm polypropylene syringe filters to remove insoluble SN-38 lactone crystals. The concentration of soluble SN-38 lactone in the filtered solutions was determined with a high performance liquid chromatographer (Shimazdu) with a fluorescence detector. The loading efficiency (L.E.) of soluble SN-38 lactone was calculated according to the equation:

$$L . E . = C / L * 100$$

Being C the concentration of SN-38 lactone in the filtered micellar system and L the theoretic load of SN-38 lactone in the final product before filtration. L.E. of the products is represented in Table 2. Products named G2B-002-20-9 and G2B-006-20-9 achieved upon filtration a final concentration of soluble SN-38 lactone of 2 mg/mL, with a loading efficiency close to 100% in both cases (Table 2). In the absence of G2B-001 or G2B-003 micelles, the same process led to insoluble SN-38 lactone in the presence of only vehicle (Table 2). Similarly, the same process led to insoluble SN-38 lactone in the presence of irinotecan at concentration 20 mg/mL in the acid solution (Table 2).

TABLE 2
SN-38 lactone solubilization parameters for G2B-001 or G2B-003 solutions at final pH 2.3-2.6

| | | | | Experimental | |
|---|---|---|---|---|---|
| | | Concentration | Theoretic | load of SN-38 | |
| | | of the | load of SN- | lactone in the | |
| | | peptide- | 38 lactone in | final filtrated | |
| Name of the | Peptide- | shuttle | the final | formulation | |
| final | shuttle | conjugate | manufacture | (mg/mL) | L.E. (%) |
| formulation | conjugate | (mg/mL) | (mg/mL) | (mean ± SD) | (mean ± SD) |
| G2B-002-20-9 | G2B-001 | 20 | 2 | 2.02 ± 0.10 | 100.9 ± 4.7 |
| G2B-002-20-16 | G2B-001 | 20 | 4 | 3.27 ± 0.36 | 81.8 ± 9.0 |
| G2B-006-20-9 | G2B-003 | 20 | 2 | 1.94 ± 0.23 | 96.8 ± 11.5 |
| SN38 in | None | 0 | 4 | 0.0053 ± 0.0030 | 0.13317 ± 0.00001 |
| Vehicle | | | | | |
| Irinotecan + | None | 0 | 2 | 0.100 ± 0.030 | 5.00 ± 1.51 |
| SN38 | | | | | |

When it is compared with the solubility data of the SN-38 liposomes or micelles disclosed in the prior art, it is also seen that the solubility data of the micelles of the present invention is also higher.

### Example 17: Visual Inspection of Turbidity of the Micellar Systems

To study the physical stability of the invented products G2B-002-20-9 and G2B-006-20-9, both carrying a final concentration of soluble SN-38 lactone of 2 mg/mL, inventors performed visual inspection of the products. Table 3 summarizes the turbidity data upon visual inspection of the products. For comparison, inventors used products obtained in the absence of G2B-001 or G2B-003 micelles, consisting of SN-38 in G2B-001 linear 20 mg/mL, SN-38 only in vehicle, or irinotecan 20 mg/mL and SN-38 in vehicle, as described in the Example “Load of soluble SN-38 lactone in the micellar system”.

TABLE 3
Visual inspection of turbidity of the SN-38 lactone-loaded
micellar systems for G2B-001 or G2B-003 solutions containing
2 mg/mL theoretic load of SN-38 lactone.

| | | Turbidity | | |
|---|---|---|---|---|
| Name of the | Peptide- | immediately | | |
| final | shuttle | after | Turbidity at | Turbidity at |
| formulation | conjugate | preparation | 1-4 weeks | 6 months |
| G2B-002-20-9 | G2B-001 | No | No | No |
| G2B-006-20-9 | G2B-003 | No | No | No |
| SN-38 in | G2B-001 | No | No | n.d.nn |
| G2B-001 | linear | | | |
| linear | | | | |
| SN-38 in | None | Yes | n.d. | n.d. |
| vehicle | | | | |
| Irinotecan + | None | Yes | n.d. | n.d. |
| SN-38 | | | | |

n.d.: not done.

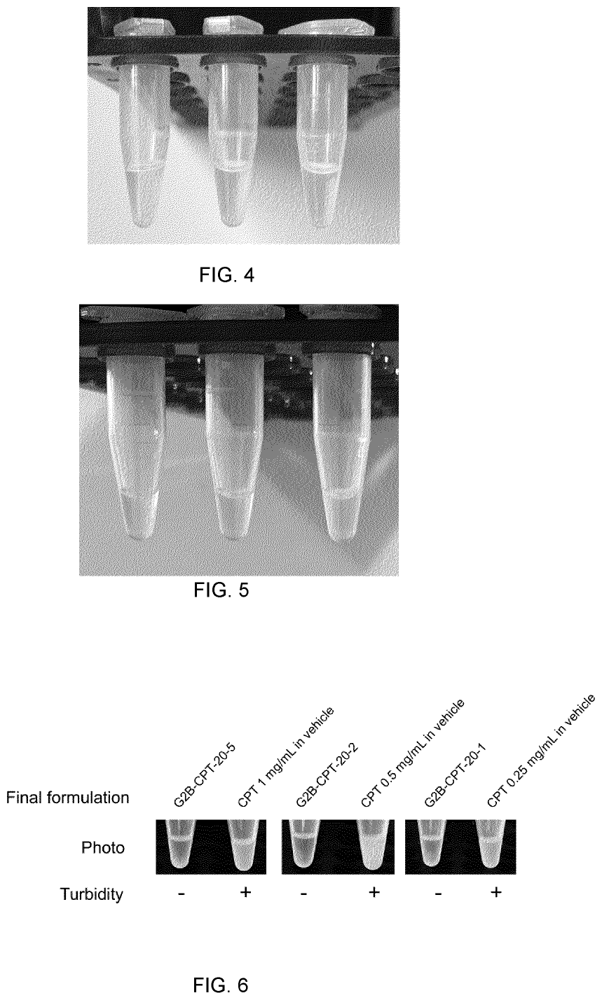

FIGS. 2-5 show photographs of products G2B-002-20-9 and G2B-006-20-9, or alternative products obtained in the absence of G2B-001 micelles, consisting of only SN-38 in vehicle, or irinotecan 20 mg/mL and SN-38 in vehicle, as described in the Example “Load of soluble SN-38 lactone in the micellar system”. The products G2B-002-20-9 and G2B-006-20-9 were not turbid, while the products including only SN-38 in vehicle and irinotecan and SN-38 showed turbidity corresponding to insoluble SN-38 lactone crystals. Photos were taken either immediately after preparation (FIGS. 2-3) or up to 6 months after preparation (FIGS. 4-5). The product including SN-38 in G2B-001 linear was not turbid at 1-4 weeks. Example 18: Visual inspection of turbidity of the micellar systems carrying water-insoluble camptothecin.

To study whether the invented systems could carry products other than SN-38, inventors selected camptothecin of formula below;

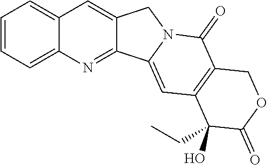 [SMILES: CC[C@@]1(O)C(=O)OCc2c1cc1n(c2=O)Cc2cc3ccccc3nc2-1]

Like SN-38, camptothecin is very insoluble as lactone, at acidic pH, and freely soluble in basic pH, as carboxylate.

G2B-001 micelles at concentration 40 mg/mL in acid solution (0.5 mL) were added to camptothecin carboxylate at concentrations 0.25, 0.5, and 1 mg/mL in basic solution (0.5 mL) in plastic vials (1.5 mL). After gentle mixing, the samples were kept at room temperature (18-22° C.). The final pH of the formulations was acid (pH=1.5), ensuring conversion to camptothecin lactone. Immediately after the preparation, and after 24 h, inventors performed visual inspection of the products. Table 4 summarizes the turbidity data upon visual inspection of the products. For comparison, inventors used the products obtained in the absence of G2B-001 micelles, consisting of camptothecin lactone only in vehicle.

TABLE 4
Visual inspection of turbidity of the micellar systems
for G2B-001 containing camptothecin lactone (CPT) at a
theoretic load (concentration of CPT) of 0.25-1 mg/mL.

| | | | Proportion w/w of | Turbidity | |
|---|---|---|---|---|---|
| Name of the | Conc. | Conc. | CPT and G2B-001 | immediately | Turbidity |
| final | G2B-001 | CPT | in the micellar | after | 24 h after |
| formulation | (mg/mL) | (mg/mL) | aqueous dispersion | preparation | preparation |
| G2B-CPT-20-5 | 20 | 1 | 4.8 | no | no |
| G2B-CPT-20-2 | 20 | 0.5 | 2.4 | no | no |
| G2B-CPT-20-1 | 20 | 0.25 | 1.2 | no | no |
| CPT in | 0 | 0.25-1 | | yes | yes |
| vehicle | | | | | |

FIG. 6 show photographs of the products detailed in Table 4. The products G2B-CPT-20-5, G2B-CPT-20-2 and G2B-CPT-20-1 were not turbid and the products including only CPT in vehicle showed turbidity corresponding to insoluble CPT crystals. Photos were taken immediately after preparation.

### Example 19: Visual Inspection of Turbidity of the Micellar Systems Carrying SN-38 Lactone and Water-Insoluble Camptothecin

To study whether the invented systems could carry SN-38 and camptothecin at the same time, inventors formulated in plastic vials (1.5 mL) G2B-001 micelles at concentration 40 mg/mL in acid solution (0.5 mL), to which they added camptothecin carboxylate at concentrations 2 or 4 mg/mL in basic solution (0.25 mL) and SN-38 carboxylate at concentrations 2 or 4 mg/mL in basic solution (0.25 mL).

After gentle mixing, the samples were kept at room temperature (18-22° C.). The final pH of the formulations was acid (pH=1.5), ensuring conversion to lactones. Immediately after the preparation, and after 24 h, inventors performed visual inspection of the products. Table 5 summarizes the turbidity data upon visual inspection of the products. For comparison, inventors used the products obtained in the absence of G2B-001 micelles, consisting of SN-38 and camptothecin lactone only in vehicle.

TABLE 5
Visual inspection of turbidity of the micellar systems for G2B-
001 containing SN-38 and camptothecin lactone (CPT) at a theoretic
load (concentration of SN-38 and CPT) of 0.5 and 1.0 mg/mL.

| | | | | Turbidity | |
|---|---|---|---|---|---|
| Name of the | Conc. | Conc. | Conc. | immediately | Turbidity |
| final | G2B-001 | CPT | SN-38 | after | 24 h after |
| formulation | (mg/mL) | (mg/mL) | (mg/mL) | preparation | preparation |
| G2B-SN38- | 20 | 1.0 | 1.0 | no | no |
| CPT-20-5-5 | | | | | |
| G2B-SN38- | 20 | 0.5 | 0.5 | no | no |
| CPT-20-2-2 | | | | | |
| SN-38 and | 0 | 0.5-1.0 | 0.5-1.0 | yes | yes |
| CPT in | | | | | |
| vehicle | | | | | |

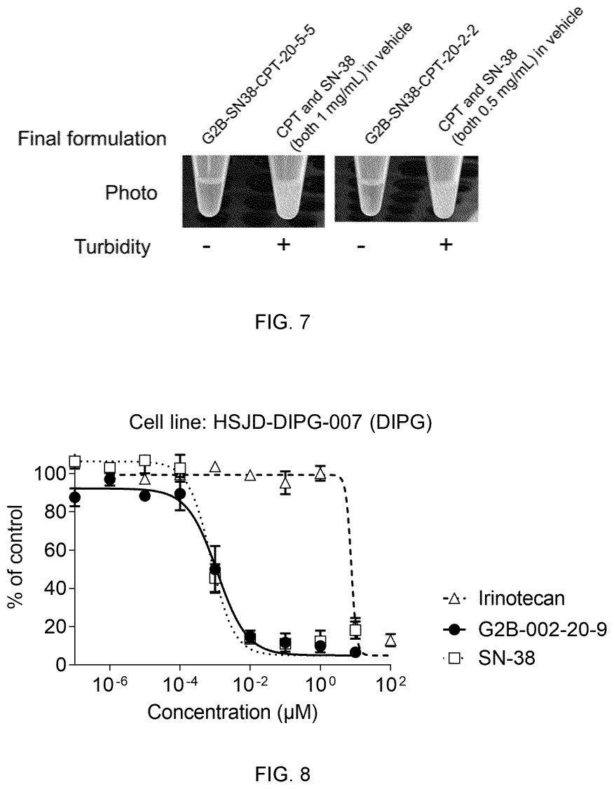

FIG. 7 shows photographs of the products formulated with G2B-001 detailes in Table 5, or alternative products obtained in the absence of G2B-001 micelles, consisting of only SN-38 and CPT in vehicle. The products G2B-SN38-CPT-20-5-5 and G2B-SN38-CPT-20-2-2 were not turbid, while the products including only SN-38 and CPT in vehicle showed turbidity corresponding to insoluble SN-38 and CPT crystals. Photos were taken immediately after preparation.

### Example 20: Stability of the Product G2B-002-20-9 Upon Lyophilization

To study whether soluble SN-38 lactone carried by the invented product G2B-002-20-9 was stable upon lyophilization, G2B-002-20-9 (280 μL in 1.5 mL plastic vials, containing 2 mg/mL of soluble SN-38 lactone) was frozen in liquid nitrogen and freeze-dried in a Telstar apparatus (LyoQuest). Upon lyophilization, dried samples were suspended in the same volume of water (280 μL). Resulting solutions showed no turbidity. Analysis of soluble SN-38 lactone by HPLC showed a recovery of soluble SN-38 lactone of 100.6±7.8% (mean and standard deviation of 4 samples prepared independently). Thus, the invented product G2B-002-20-9 was stable upon lyophilization and reconstitution in water.

### Example 21: In Vitro Activity of the Product G2B-002-20-9

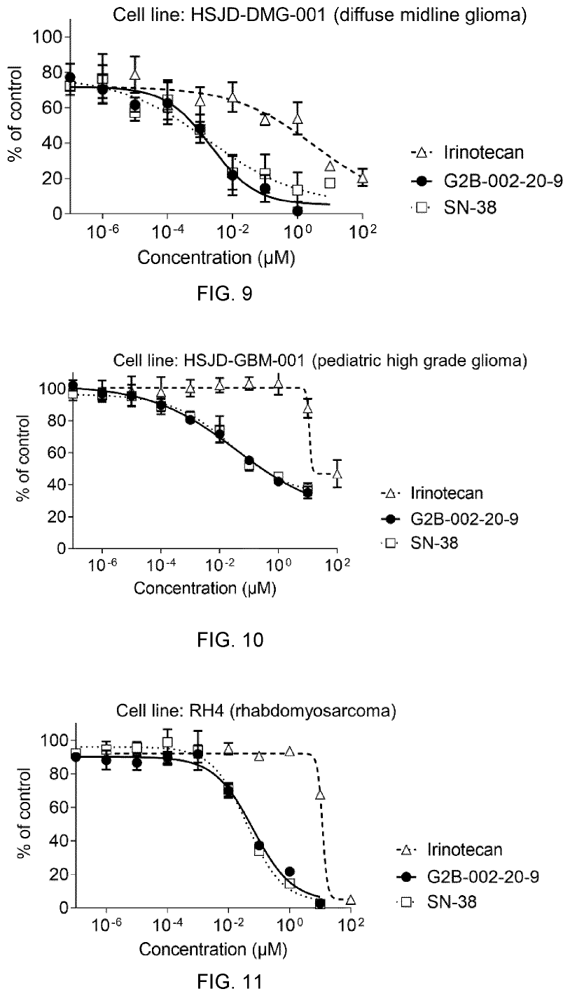

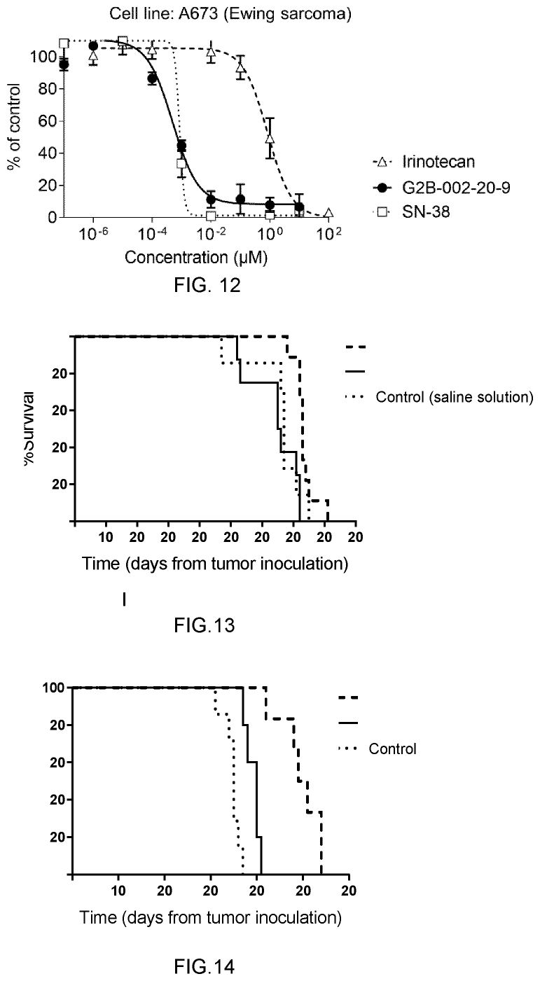

The activity of the product G2B-002-20-9 carrying soluble SN-38 lactone was compared with the one of free SN-38 lactone and irinotecan. Cancer cell lines were obtained from the repository maintained at Hospital Sant Joan de Deu (Barcelona, Spain). Cancer cell lines used in these experiment included cancer types of pediatric gliomas including diffuse intrinsic pontine glioma (HSJD-DIPG-007), pediatric diffuse midline glioma (HSJD-DMG-001) and pediatric high-grade glioma (HSJD-GBM-001), and cells lines of other pediatric solid tumors such as Ewing sarcoma (A673) and rhabdomyosarcoma (RH4). Briefly, between 3000 and 30,000 cancer cells were cultured in 96 well plates and 24 h later the cells were exposed to compound G2B-002-20-9 (concentration range of soluble SN-38 lactone ranging 10-0.0000001 μM), SN-38 lactone (concentration range 10-0.0000001 μM), or irinotecan (concentration range 100-0.000001 μM). To add the drugs to the cells in culture, G2B-002-20-9, irinotecan and SN-38 were prepared in culture medium from stock solutions. The tetrazolium compound [3-(4,5-dimethylthiazol-2-yl)-5-(3-carboxymethoxyphenyl)-2-(4-sulfophenyl)-2H-tetrazolium](MTS assay; Promega, Fitchburg, WI) was used to determine cell viability after 72 h incubation with the drugs. The concentrations of drug required to cause a reduction of 50% in cell proliferation (IC50 and 95% confidence intervals) were calculated with Graphpad Prism 8 software (Lₐ Jolla, CA). Results are expressed as the percentage of cell viability compared to untreated, control wells. The activity of G2B-002-20-9 carrying SN-38 lactone was equivalent to the activity of free SN-38 lactone, and superior to irinotecan, in all tested cell lines. FIG. 8 shows results for HSJD-DIPG-007 cells. FIG. 9 shows results for HSJD-DMG-001 cells. FIG. 10 shows results for HSJD-GBM-001 cells. FIG. 11 shows results for RH4 cells. FIG. 12 shows results for A673 cells.

### Example 22: Antitumor Efficacy of the Invented Product in DIPG and pHGG Xenografts

The activity of the product G2B-002-20-9 and the closely related drug irinotecan, administered intravenously in mice bearing intracranial human brain tumors was compared. Inventors established brain tumor xenografts in immunodeficient athymic nude mice by the intracranial injection of half million cells in the fourth ventricle of anesthetized mice, using a stereotactic apparatus. Two different xenografts were used. Xenograft HSJD-DIPG-007 is a clinically relevant model of DIPG, holding the mutations H3.3-K27M and ACVR1-R206H. Xenograft HSJD-GBM-001 is a relevant model of pHGG, holding wild type H3.3 and mutation p.G245S in the gene TP53. The goal of this experiment was to study whether the activity of the new compound G2B-002-20-9, which carries soluble SN-38 lactone and penetrates the blood-brain barrier, was superior to the one of irinotecan, which is metabolized to SN-38 lactone by carboxylesterases upon administration in mouse blood, and does not penetrate significantly the blood-brain barrier. HSJD-DIPG-007 cells were injected in 25 mice, and HSJD-GBM-001 cells in 21 mice. Treatment consisted of intravenous injections through the tail vein. Treatment started 31 days post-inoculation of HSJD-DIPG-007 cells, and 8 days post-inoculation of HSJD-GBM-001 cells. Treatment days with G2B-002-20-9 were 1, 2, 3, 4, 5, 8, 9, 10, 11, and 12 for HSJD-DIPG-007-bearing mice, and 1, 2, 3, 4, 5, 8, 9, 10, 11, 12, 15, 16, 17, 18, 19, 22, 23, 24, 25 and 26 for HSJD-GBM-001-bearing mice. Treatment days for irinotecan were 1, 2, 3, 4, and 5 for HSJD-DIPG-007-bearing mice, and 1, 2, 3, 4, 5, 8, 9, 10, 11, 12, 15, 16, 17, 18, 19, 22, 23, 24, 25 and 26 for HSJD-GBM-001-bearing mice. For mice bearing HSJD-DIPG-007 tumors, treatment groups received either G2B-002-20-9 10 mg/kg (dose of the soluble SN-38 lactone), irinotecan 40 mg/kg, or control (saline solution). A minimum of eight mice received each treatment. For mice bearing HSJD-GBM-001 tumors, treatment groups received either G2B-002-20-9 10 mg/kg (dose of the soluble SN-38 lactone), irinotecan 10 mg/kg, or saline control (saline solution). Seven mice received each treatment. In the experiment with HSJD-DIPG-007-bearing mice, G2B-002-20-9 was safely administered. During treatment, weight loss after 10 doses of G2B-002-20-9 was −7.3±3.5% (mean and standard deviation), with no toxic deaths. Irinotecan treatments at 40 mg/kg caused one toxic death and weight loss of −16.4±9.4%. Upon cessation of treatments, survival curves obtained in the study are shown in FIG. 13. Mice treated with G2B-002-20-9 survived significantly longer time (median survival=73 days) than control mice (median survival=67 days; P=0.0344) and irinotecan (median survival=65.5 days; P=0.0009), while irinotecan did not improve animal survival compared to control (P=0.5851). In the experiment with HSJD-GBM-001-bearing mice, all mice tolerated treatments without significant weight loss. Upon cessation of treatments, survival curves obtained in the study are shown in FIG. 14. Mice treated with G2B-002-20-9 survived significantly longer time (median survival=50 days) than control mice (median survival=35 days; P=0.0003) and irinotecan (median survival=40 days; P=0.008). Irinotecan improved 5 days animal survival compared to control (P=0.0020). Overall, these experiments demonstrate that G2B-002-20-9 is superior to irinotecan in the treatment of mice bearing intracranial DIPG and pHGG.

### Example 23: Antitumor Efficacy of the Invented Products in Subcutaneous Patient-Derived Xenografts

The inventors compared the activity of the new compounds G2B-002-10-5, G2B-002-20-9 and G2B-002-20-16 with the one of the closely related drug irinotecan, administered intravenously in mice bearing subcutaneous human tumors. Patient-derived xenograft (PDX) tissues were obtained from immunodeficient athymic nude mice at Hospital Sant Joan de Deu (Barcelona, Spain). PDX were established from biopsies of pediatric patients with Ewing sarcoma, neuroblastoma, osteosarcoma and rhabdomyosarcoma, as detailed in one publication (1). Identification of the PDXs is in Table 6.

TABLE 6
Identification of the PDX models included in the
study of antitumor efficacy of the new compounds

| Identification | |
|---|---|
| of the PDX | Tumor |
| HSJD-aRMS-21 | Rhabdomyosarcoma |
| HSJD-NB-016 | Neuroblastoma |
| HSJD-ES-29 | Ewing sarcoma |
| HSJD-OS-12 | Osteosarcoma |
| HSJD-NB-013 | Neuroblastoma |

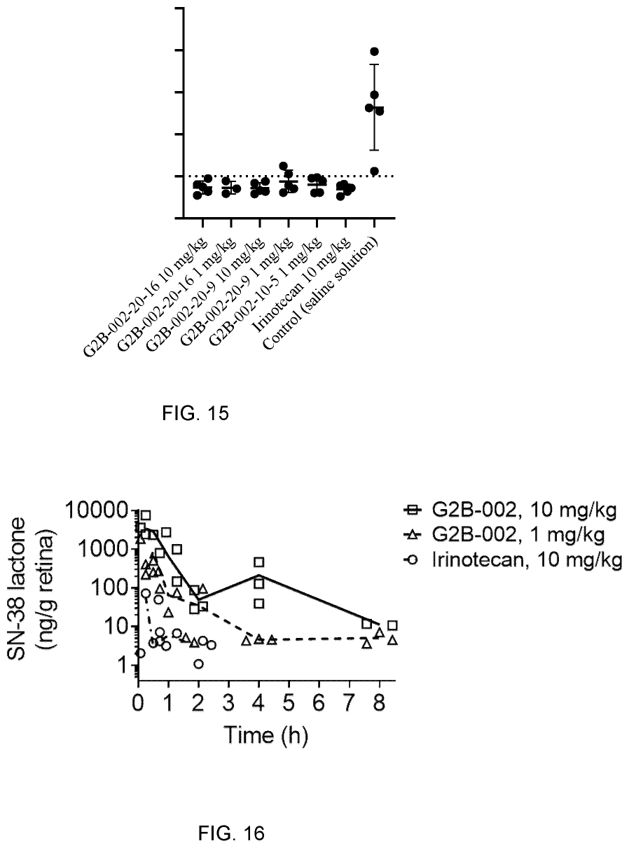

The goal of this experiment was to study whether the activity of the new products, who carry SN-38 lactone, was comparable to the one of irinotecan, who is metabolized to SN-38 lactone by carboxylesterases upon administration in mouse blood. Each PDX was inserted subcutaneously in 7-8 mice. Treatment consisted of intravenous injections through the tail vein. Treatment started when the volume of subcutaneous tumors was in the range 100-300 mm3. Treatment days were 1, 2, 3, 4, 5, 8, 9, 10, 11, and 12. We included seven treatment groups in the study: G2B-002-20-16 at 10 mg/kg, G2B-002-20-16 at 1 mg/kg, G2B-002-20-9 at 10 mg/kg, G2B-002-20-9 at 1 mg/kg, G2B-002-10-5 at 1 mg/kg, irinotecan at 10 mg/kg, and saline control (saline solution). One mouse received each treatment. All mice tolerated the treatments without significant weight loss. At day 15, inventors measured the volume of the tumors and calculated the proportion between the final volume after treatment and the initial volume at day 1. All tumors treated with irinotecan and G2B-002 products were reduced significantly in volume compared to control tumors treated with saline (P=0.0001; paired one-way ANOVA with Dunnett's multiple comparison test) (FIG. 15). Overall, these experiments demonstrate that G2B-002-20-16, G2B-002-20-9 and G2B-002-10-5 are similar to irinotecan in the treatment of mice bearing extracranial Ewing sarcoma, neuroblastoma, osteosarcoma and rhabdomyosarcoma.

### Example 24: Distribution of SN-38 Lactone in the Mouse Retina after Intravenous Administration of G2B-002-20-9 and Irinotecan

Inventors evaluated the distribution of SN-38 lactone in the retina after the intravenous administration of G2B-002-20-9, at dosages enough to provide 10 mg/kg and 1 mg/kg of free SN-38 lactone, or irinotecan at 10 mg/kg. Inventors administered intravenously G2B-002-20-9, amount enough to provide 10 mg/kg or 1 mg/kg of free SN-38 lactone, in 18 athymic nude mice each dosage level, and irinotecan at 10 mg/kg, in another 12 mice. At time points 0.25, 0.5, 1, 2, 4 and 8 hours for the groups receiving G2B-002-20-9, and time points 0.25, 0.5, 1 and 2 hours for the group receiving irinotecan, they sacrificed the animals (three at each time point) by decapitation, dissected the retinae and stored the samples frozen until analysis using published methods.

After the administration of G2B-002-20-9 at dosage enough to provide 10 mg/kg of free SN-38 lactone, mean maximum concentration of SN-38 lactone in the retinae was 2878 ng/g (range 2330-3593), achieved at time point 0.5 hours. For this dose level, the concentration of SN-38 lactone remained above 100 ng/g at least 4 hours (FIG. 16).

After the administration of G2B-002-20-9 at dosage enough to provide 1 mg/kg of free SN-38 lactone, mean maximum concentration of SN-38 lactone in the retinae was 867 ng/g (range 254-1844), achieved at time point 0.5 hours. For this dose level, the concentration of SN-38 lactone remained above 100 ng/g at least 0.5 hours (FIG. 16).

After the administration of irinotecan at 10 mg/kg, the concentration of SN-38 lactone remained below 100 ng/g at all time points. Mean maximum concentration of SN-38 lactone in the retinae was 57 ng/g (range 48-72 ng/g), achieved at time point 0.25 hours. After 2 hours, SN-38 lactone was below the limit of detection (5 ng/g) in this group (FIG. 16).

Overall, these experiments show that the new invention improves retinal distribution of soluble SN-38 lactone.

### Example 25: In Vitro G2B-002-20-9 Micelles Transport Across Human BBB Model

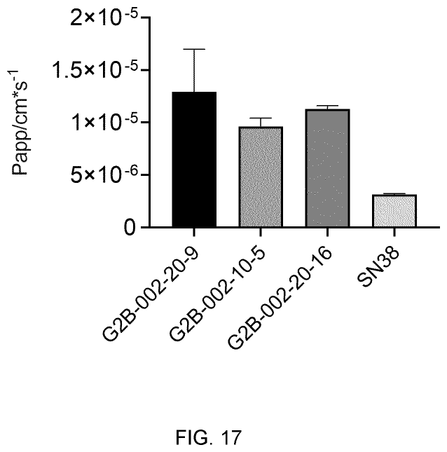

It is well described that irinotecan and SN-38 cannot cross the BBB in vivo. (Pharmaceutics 2020, 12 399 and Cancer Res 1993, 53(12) 2823-9. Using an in vitro BBB human model, all compounds were evaluated at 100 μM in Ringer Hepes. The time assay was 2 h at 37° C. Acceptors and time 0 were analyzed by UPLC. All evaluated by triplicate and Lucifer Yellow (LY) at 25 μM. As mentioned in the article ACIE, 2015,3967 when Papp permeabilities are higher than for natural ligands such as Tf 0.953 10⁻⁶ then transport is considered relevant. Many times BBBshuttle-cargo conjugates have lower Paap than BBBshuttles alone, as they are shuttling a cargo. The results are shown in FIG. 17.

### Example 26: Distribution of SN-38 Lactone in Mouse Brain and Cerebrospinal Fluid after Intravenous Administration of G2B-002-20-9 and Irinotecan

Inventors evaluated the distribution of SN-38 lactone in brain and cerebrospinal fluid (CSF) one hour after the intravenous administration of G2B-002-20-9, at a dosage enough to provide 10 mg/kg of free SN-38 lactone, or irinotecan at 40 mg/kg. Inventors administered intravenously G2B-002-20-9, amount enough to provide 10 mg/kg of free SN-38 lactone, in 4 athymic nude mice, and irinotecan at 40 mg/kg, in another 4 mice. One hour later, they anesthetized mice and obtained 5 μL of CSF with a glass capillary. Then, they sacrificed the animals by decapitation, dissected the brains and stored the samples frozen until analysis using published methods. In brain, SN-38 lactone was not detectable, i.e., its concentration was lower than the limit of quantification 5 ng/g, one hour after the administration of irinotecan at 40 mg/kg. After the administration of G2B-002-20-9, mean SN-38 concentration in brain was 33 ng/g (range 13-60 ng/g). In CSF, SN-38 lactone achieved a mean concentration 0.5 ng/mL (range 0.2-0.8 ng/mL), after the administration of irinotecan at 40 mg/kg. After the administration of G2B-002-20-9, SN-38 lactone achieved a median concentration of 61 ng/mL (range 33-97 ng/mL). Overall, these experiments show that the new invention improves CNS distribution of soluble SN-38 lactone. In the case of CSF, the increase of SN-38 lactone distribution is around 100 times.

### CITATION LIST

### Patent Literature

EP2644191B1
U.S. Pat. No. 8,299,089B2
WO2015/051307A1
CN10206099A1
CN110124052A

### Non Patent Literature

Sosnik, Drug self-assembly: A phenomenon at the nanometer scale with major impact in the structure-biological properties relationship and the treatment of disease, Prog Mater Sci, 2016, vol. 82, pp. 39-82
F Meyer-Losic et al in “DTS-108, A novel Peptidic prodrug of SN-38: In vivo Efficacy and Toxicokinetic Studies”, Clinical Cancer Research, 2008, vol. 14, issue 7, pp. 2145-2153
A. Buckin et al., in “Amphiphilic Polymeric Nanoparticles Modified with a Protease-Resistant Peptide Shuttle for the Delivery of SN-38 in Diffuse Intrinsic Pontine Glioma” ACS Applied Nano Materials, 2021, vol. 4, issue 2, pp. 1314-1329
Ri, Masaki et al.; in “A phase I/II study for dose-finding, and to investigate the safety, pharmacokinetics and preliminary efficacy of NK012, an SN-38-incorporating macromolecular polymeric micelle, in patients with multiple myeloma “Internal Medicine (Tokyo, Japan) (2018), vol. 57(7), pp. 939-946
Lei, Fan et al:, Nanoscale platform for delivery of active IRINOX to combat pancreatic cancer Journal of Controlled Release (2021), vol. 330, pp. 1229-1243
Zhang et al., “Development and characterization of a novel liposome-based formulation of SN-38” International Journal of Pharmaceutics, 2004, vol. 270, pp. 93-107
M. J. Hatfield and P. M. Potter, Exp. Opin. Ther. Pat. 2011, vol. 21(8), pp. 1159-1171
A. Madder et al., Eur. J. Org. Chem,. 1999, pp. 2787-2791
Barker et al. J. Med. Chem., 1992, vol. 35, pp. 2040-2048.
Greene's Protective Groups in Organic Synthesis, Fifth Edition. Peter G. M. Wuts. 2014 John Wiley & Sons, Inc. Published 2014 by John Wiley & Sons, Inc. pp. 26-29, 271-279, 456-463, 69-77, 371-374
M. Kazemi & L. Shiri, Journal of Sulfur Chemistry, 2015, vol. 36:6, pp. 613-623.
M. T. Nguyen et al., J. Org. Chem. 1998, 63, vol. 20, pp. 6878-6885.
F. David et al., Org. Process Res. Dev. 2010, vol. 14, 4, pp. 999-1007.
S. F. M. van Dongen et al.; Bioconjugate Chem., 2009, vol. 20, pp. 20-23.
J. H. Lee et al. Eur. J. 30 Biochem., 2001, vol. 268. pp. 2004-2012
M. Amblard, et al., “Methods and protocols of modern solid-phase peptide synthesis. Molecular Biotechnology 2006, vol. 33, pp. 239-254
J. Med. Chem. 2008, 51, 21,6916-6926.

1. A micelle comprising a peptide conjugate of SN-38 and one or more free therapeutic active agents which anticancer activity,
wherein:
the micelle is a core-shell structure comprising an inner core and an external shell wherein the free therapeutic active agent is loaded in the inner core and the peptide conjugate of SN-38 forms the external shell;
the peptide conjugate of SN-38 is a compound of formula (I) or a pharmaceutically acceptable salt thereof, 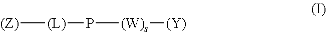
wherein:
Z is a radical of the pharmaceutical active ingredient SN-38 or a pharmaceutically acceptable salt thereof, wherein the pharmaceutical active ingredient SN-38 has formula (II), and wherein Z is attached to a linker L independently by only one of the two hydroxyl groups (a) or (b) of the pharmaceutical active ingredient; 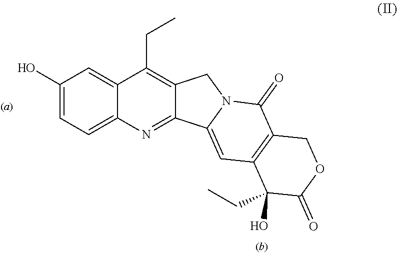 [SMILES: CCc1c2c(nc3ccc(O)cc13)-c1cc3c(c(=O)n1C2)COC(=O)[C@]3(O)CC]
L is a linker which is a biradical composed from 2 to 8 biradicals L′ and has the formula: 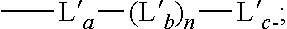
Lₐ′ is a biradical selected from the group consisting of: —C(═O)—(CH₂)ᵣ—C(═O)—; —C(═O)—(CH₂)ᵣ—NH—; —C(═O)—(CH₂)ᵣ—S—; —C(═O)—(CH₂)ᵣ—O—; —C(═O)—NH—(CH₂)ᵣ—C(═O)—; —C(═O)—NH—(CH₂)ᵣ—NH—; —C(═O)—NH—(CH₂)ᵣ—S—; —C(═O)—NH—(CH₂)ᵣ—O—; —(CH₂)ᵣ—C(═O)—; —(CH₂)ᵣ—NH—; —(CH₂)ᵣ—S—; —(CH₂)ᵣ—O—; —Si(R₁)(R₂)—(CH₂)ᵣ—NH—; —Si(R₁)(R₂)—(CH₂)ᵣ—C(═O)—; —Si(R₁)(R₂)—(CH₂)ᵣ—O—; —Si(R₁)(R₂)—(CH₂)ᵣ—S—; —SO₂—(CH₂)ᵣ—NH—; —SO₂—(CH₂)ᵣ—C(═O)—; —SO₂—(CH₂)ᵣ—O—; —SO₂—(CH₂)ᵣ—S—; —P(═O)(OR₁)—O—(CH₂)ᵣ—NH—; —P(═O)(OR₁)—O—(CH₂)ᵣ—C(═O); —P(═O)(OR₁)—O—(CH₂)ᵣ—O—; —P(═O)(OR₁)—O—(CH₂)ᵣ—S—; —CH(OH)—(CH₂)ᵣ—NH—; —CH(OH)—(CH₂)ᵣ—C(═O)—; —CH(OH)—(CH₂)ᵣ—O—; —CH(OH)—(CH₂)ᵣ—S—; 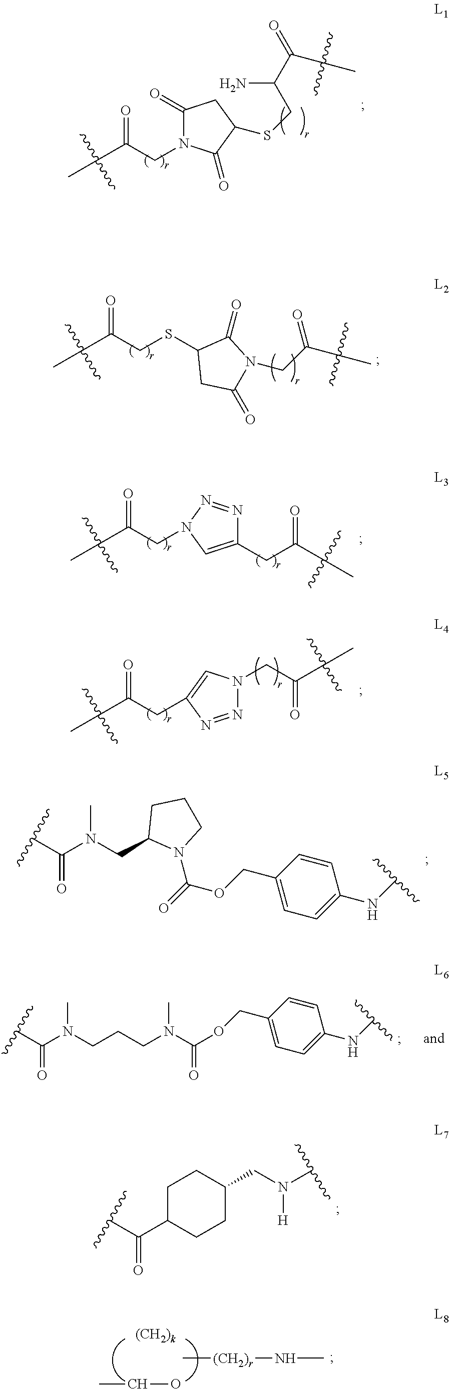 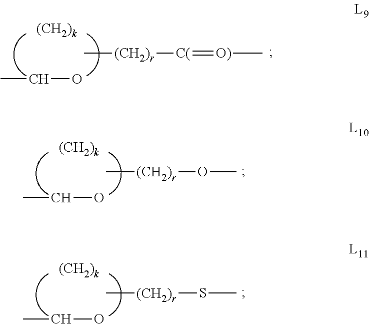
the substituent in any of L₈-L₁₁ can be in any position of the cycles; 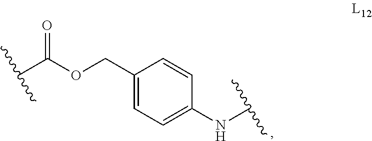 [SMILES: *Nc1ccc(COC(*)=O)cc1]
L_{b}′ is a biradical independently selected from the group consisting of: —NH—(CH₂)ᵣ—C(═O)—; —C(═O)—(CH₂)ᵣ—C(═O)—; —S—(CH₂)ᵣ—C(═O)—; —O—(CH₂)ᵣ—C(═O)—; —NH—(CH₂)ᵣ—; —C(═O)—(CH₂)ᵣ—; —S—(CH₂)ᵣ—; —O—(CH₂)ᵣ—; —NH—CH—((CH₂)ᵣ—NH₂)—C(═O)—; —S—CH₂—CH(NH₂)—C(═O)—; —(CH₂)ᵣ—C(═O)—; —(CH₂)ᵣ—O—; —(CH₂)ᵣ—NH—; —(CH₂)ᵣ—S—; —C(═O)—(CH₂)ᵣ—NH—; —C(═O)—(CH₂)ᵣ—O—; —C(═O)—(CH₂)ᵣ—S—; —NH—(CH₂)ᵣ—O—; —NH—(CH₂)ᵣ—NH—; —NH—(CH₂)ᵣ—S—; and combinations thereof; 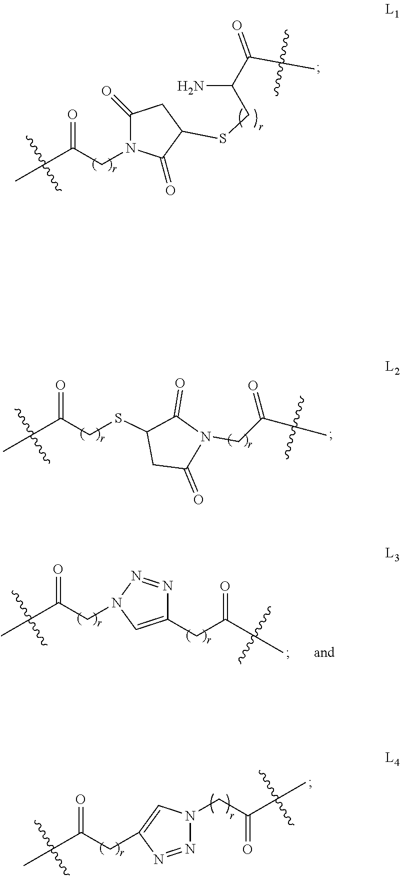
L_{c}′ is a biradical selected from the group consisting of:=NH—(CH₂)ᵣ—C(═O)—; —NH—CH—((CH₂)ᵣ—NH₂)—C(═O)—; —C(═O)—(CH₂)ᵣ—C(═O)—; —S—(CH₂)ᵣ—C(═O)—; —S—CH₂—CH(NH₂)—C(═O)—; —O—(CH₂)ᵣ—C(═O)—, —(CH₂)ᵣ—C(═O)—; 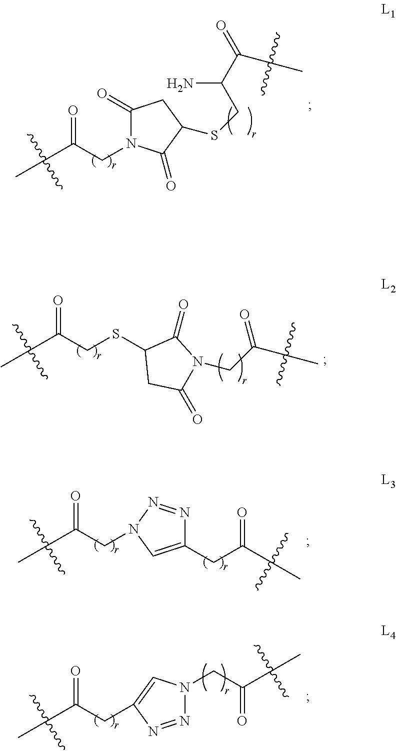 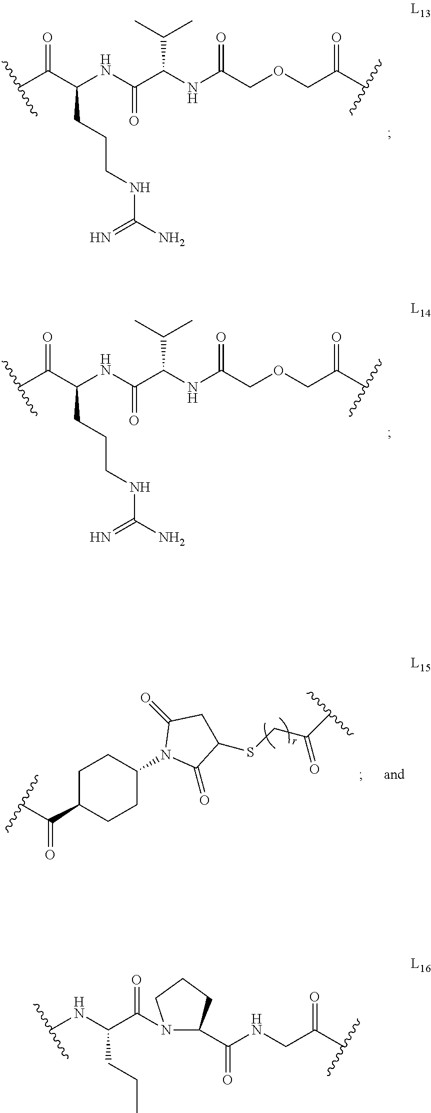
P is a biradical of a peptide selected from the group consisting of:
(a) a peptide which comprises the amino acid sequence X₁KAPETALX₂ with an intrapeptide bond between the X₁ and X₂ which is an amide bond; wherein X₁ is selected from the group consisting of Dap and Dab; and X₂ is selected from the group consisting of D (aspartic acid) and E (glutamic acid); 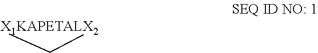
(b) a peptide having 12-20 amino acids residues in length having at least an intrapeptide bond
which is a disulfide or diselenide bond, and comprises an amino acid sequence which is: X₃KAPETALX₄AAA; having at least an intrapeptide disulfide or diselenide bond between X₃ and X₄, wherein X₃ and X₄ are equal and are selected from the group consisting of C (cysteines), Sec (selenocysteines), and Pen (penicillamines); 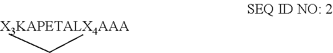
(c) a peptide having 9-11 amino acids residues in length having at least an intrapeptide bond which is a disulfide or diselenide bond and consists of an amino acid sequence selected from the group consisting of X₅KAPETALX₆; X₅KAPETALX₆A; and X₅KAPETALX₆AA having at least an intrapeptide disulfide or diselenide bond between X₅ and X₆; wherein X₅ and X₆ are equal and are selected from the group consisting of C (cysteines), Sec (selenocysteines), and Pen (penicillamines); 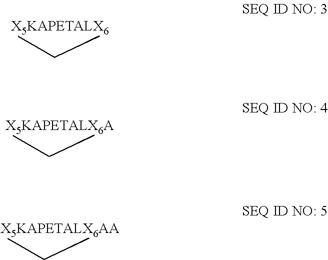
(d) a peptide which has 16 amino acid residues and comprises the amino acid sequence X₇NX₈KAPETALX₉AAAX₁₀H with an intrapeptide disulfide or diselenide bond between the X₇ and X₉, and between X₅ and X₁₀; wherein X₇-X₁₀ are independently selected from the group consisting of C (cysteines), Sec (selenocysteines), and Pen (penicillamines); provided that X₇ and X₉ are equal, and X₈-X₁₀ are equal; 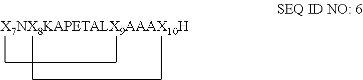
and (e) peptide which comprises the amino acid sequence X₁KAPETALX₂ wherein X₁ is selected from the group consisting of Dap and Dab; and X₂ is selected from the group consisting of D (aspartic acid) and E (glutamic acid) (SEQ ID NO:7), being a lineal peptide;
W is a biradical selected from the group consisting of —NH—(CH₂)ᵣ—C(═O)—, and —NH—CH((CH₂)ᵣ—NH₂)—C(═O)—;
Y is a radical is selected from the group consisting of —NH₂, —OH, —OR₃, and —NHR₃;
s is an integer independently selected from 0 to 1;
n is an integer from 0 to 6;
r is an integer independently selected from 1 to 5;
k is an integer from 5 to 8;
R₁ and R₂ are independently selected from an (C₁-C₆)-alkyl;
R₃ is a radical selected from the group consisting of (C₁-C₆)-alkyl;
Lₐ′ is attached to the radical Z through a bond which is selected from the group consisting of an ester, ether, urethane, silyl ether, sulphonate, phosphate, ketal, hemiketal, carbonate, and carbamate bond, the bond being formed between the C═O, SO₂, Si, P, CH or CH₂ groups on the left side of the draw Lₐ formulas and one of the hydroxyl groups of the SN-38;
when n=0, Lₐ′ is attached to the radical L_{c}′ through a chemically feasible bond which is selected from the group consisting of amine, amide, ether, thioether, disulfide, ester, and thioester, the bond being formed between the functional groups on the right side of the draw Lₐ′ formulas and the functional groups of the left side of the L_{c}′ formulas;
when n=1, Lₐ′ is attached to the radical L_{b}′ through a chemically feasible bond which is selected from the group consisting of amine, amide, ether, thioether, disulfide ester, and thioester, the bond being formed between the functional groups on the right side of the draw Lₐ′ formulas and the functional groups on the left side of the L_{b}′ formulas; and L_{b}′ is attached to the radical L_{c}′ through a chemically feasible bond which is selected from the group consisting of amine, amide, ether, thioether, disulfide, ester, and thioester, the bond being formed between the functional groups on the right side of the draw L_{b}′ formulas and the functional groups on the left side of the draw L_{c}′ formulas;
when n is higher than 1, L_{b}′ are equal or different and are attached among them through a chemically feasible bond selected from the group consisting of amine, amide, ether, thioether, disulfide, ester, and thioester; being one L_{b}′ terminal attached to Lₐ′ through a chemically feasible bond which is selected from the group consisting of amine, amide, ether, thioether, disulfide, ester, and thioester, the bond being formed between the functional groups on the right side of the draw Lₐ′ formulas and the functional groups of the left side of the draw L_{b}′ formulas; and being another L_{b}′ terminal attached to L_{c}′ through a chemically feasible bond which is selected from the group consisting of amine, amide, ether, thioether, disulfide, ester, and thioester, the bond being formed between the functional group on the right side of the draw L_{b}′ formulas and the functional group on the left side of the draw L_{c}′ formulas;
L_{c}′ is attached to the biradical P through an amide bond formed with the carbonyl group on the right side of the draw L_{c}′ formulas and an amino group of the first amino acid of the peptide sequence P;
when s=0, P is directly attached to Y through an amide, carboxylic acid or ester bond, the bond being formed between the C═O of the C-terminal of the last amino acid of the sequence P, and the radical Y which is —NH₂, —OH, —OR₃, or —NHR₃; and
when s=1, P is attached to a radical W through an amide bond formed with a C═O of the C-terminal of the last amino acid of the sequence P, the bond being formed between the functional groups on the left side of the draw W formulas and the functional groups (C═O) of the C-terminal of the last amino acid of the sequence P on the right side of the draw sequence; and W is attached to Y as follows: —C(═O)—NH—(CH₂)ᵣ—C(═O)—Y, or —C(═O)—NH—CH((CH₂)ᵣ—NH₂)—C(═O)—Y.

2. The micelle according to claim 1, wherein the drug in the inner core is SN-38 lactone.

3. The micelle according to claim 1, wherein in the peptidic conjugate of SN-38 of formula (I), P is a biradical of a peptide selected from the group consisting of:
(a) a peptide which comprises the amino acid sequence DapKAPETALD with an intrapeptide bond between the Dap and D which is an amide bond, that is SEQ ID NO:8: 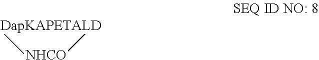
(b) a peptide having 9-20 amino acids residues in length having at least an intrapeptide bond, which is a disulfide bond, and comprises an amino acid sequence which is: CKAPETALCAAA having at least an intrapeptide disulfide bond between cysteines 1 and 9, that is 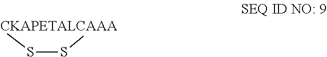
(c) a peptide having 9-11 amino acids residues in length having at least an intrapeptide bond which is a disulfide bond and consists of an amino acid sequence selected from the group consisting of CKAPETALC; CKAPETALCA; and CKAPETALCAA having at least an intrapeptide disulfide bond between cysteines 1 and 9, that are 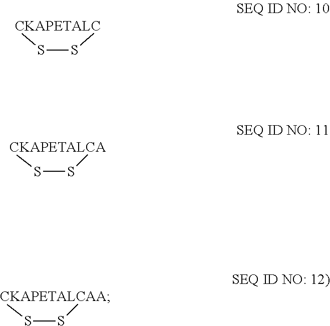
and
(d) a peptide which has 16 amino acid residues and comprises the amino acid sequence CNCKAPETALCAAACH with an intrapeptide disulfide bond between the first and third cysteine which are cysteines 1 and 11, and between the second and the fourth cysteine which are cysteine 3 and 15, that is, 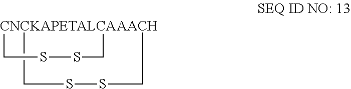
(e) a peptide which comprises the amino acid sequence DapKAPETALD (SEQ ID NO:14).

4. The micelle according to claim 3, wherein in the peptidic conjugate of SN-38 of formula (I), P is a biradical of a peptide selected from the group consisting of:
(a) the peptide having the amino acid sequence DapKAPETALD with an intrapeptide bond between the Dap and D which is an amide bond (SEQ ID NO:8);
(b) the peptide having the amino acid sequence CKAPETALC having at least an intrapeptide disulfide bond between cysteines in position 1 and 9 (SEQ ID NO:10); and
(c) the peptide having the amino acid sequence DapKAPETALD (SEQ ID NO:14).

5. The micelle according to claim 4, wherein in in the peptidic conjugate of SN-38 of formula (I), P is a biradical of the peptide DapKAPETALD with an intrapeptide bond between the Dap and D which is an amide bond (SEQ ID NO:8).

6. The micelle according to claim 1, wherein in the peptidic conjugate of SN-38 of formula (I),
Lₐ′ is a biradical selected from the group consisting of: —C(═O)—(CH₂)ᵣ—C(═O)—, —C(═O)—(CH₂)ᵣ—NH—, —C(═O)—(CH₂)ᵣ—S—; —C(═O)—(CH₂)ᵣ—O—; —C(═O)—NH—(CH₂)ᵣ—C(═O)—; L₁, L₂, L₃, L₄, L₅, L₆, L₇, and L₁₂.

7. The micelle according to claim 1, wherein in the peptidic conjugate of SN-38 of formula (I), L is a linker selected from the group consisting of:
a) Lₐ′ is L₃ of formula below, L_{b}′ is selected from the group consisting of —NH—(CH₂)ᵣ—O—, —(CH₂)ᵣ—O—; and —(CH₂)ᵣ—NH—, and combinations thereof, and L_{c}′ is —C(═O)—(CH₂)ᵣ—C(═O)—, 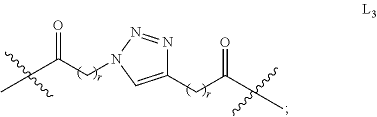
b) a biradical composed from 2 biradicals, n=0, Lₐ′ is L₁₂, and L_{c}′ is L₁₃; and
c) a biradical composed from 2 biradicals, n=0, Lₐ′ is —C(═O)—NH—(CH₂)ᵣ—C(═O)—, and L_{c}′ is L₁₅.

8. The micelle according to claim 7, wherein the peptidic conjugate of SN-38 of formula (I) is a compound selected from the group consisting of:

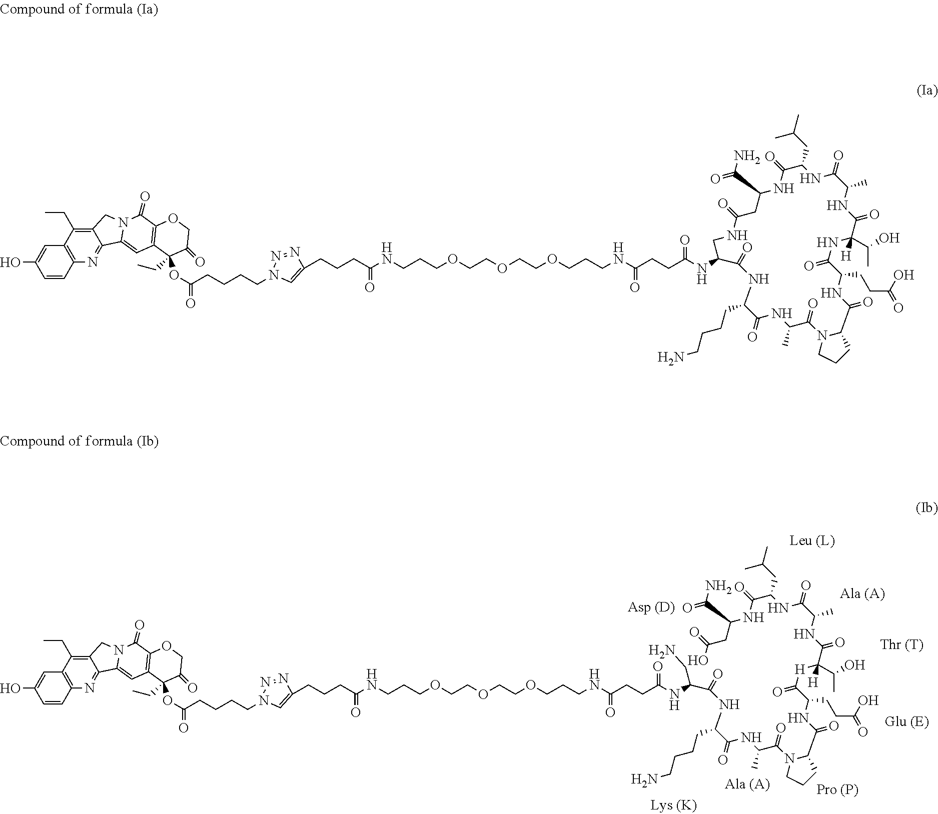

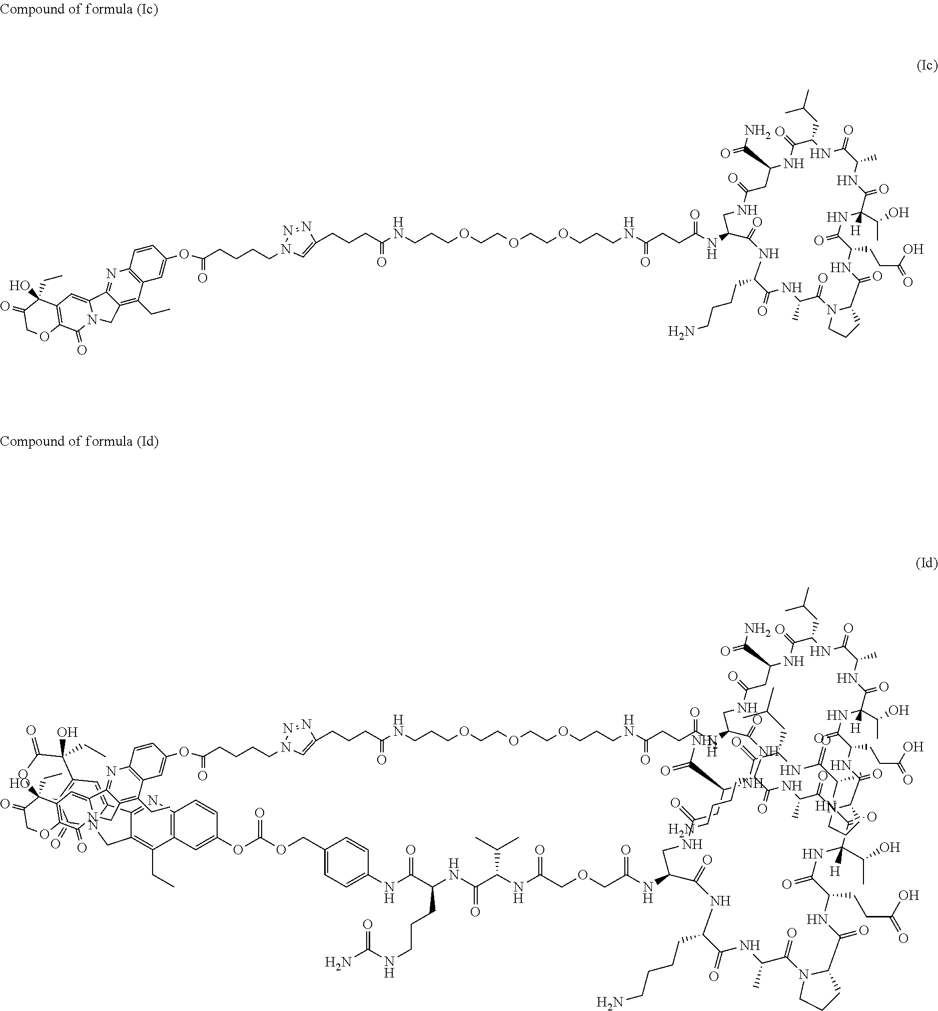

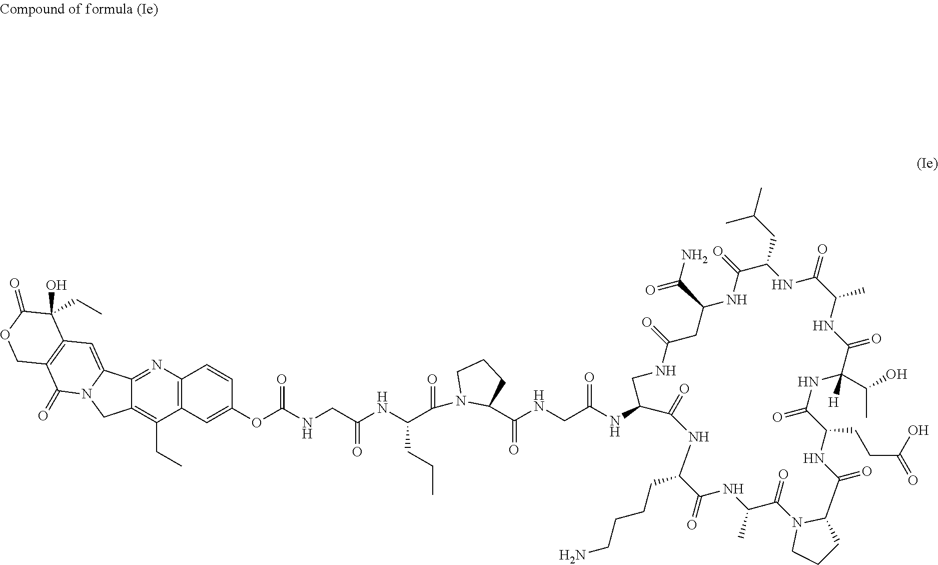 [SMILES: CCC[C@H](NC(=O)CNC(=O)Oc1ccc2nc3c(c(CC)c2c1)Cn1c-3cc2c(c1=O)COC(=O)[C@]2(O)CC)C(=O)N1CCC[C@H]1C(=O)NCC(=O)N[C@H]1CNC(=O)C[C@@H](C(N)=O)NC(=O)[C@H](CC(C)C)NC(=O)[C@H](C)NC(=O)[C@H]([C@@H](C)O)NC(=O)[C@H](CCC(=O)O)NC(=O)[C@@H]2CCCN2C(=O)[C@H](C)NC(=O)[C@H](CCCCN)NC1=O]

9. The micelle according to claim 1, which is in form of an aqueous dispersion of micelles as defined in claim 1, in which the free therapeutic anticancer drug is different.

10. The micelle according to claim 9, wherein the concentration of the peptide conjugate of SN-38 in the micellar aqueous dispersion is up to 50 mg/ml, the free therapeutic anticancer active agent is SN-38, and the concentration of free SN-38 is up to 25 mg/ml.

11. The micelle according to claim 10, which is selected from the group consisting of:
a) a micelle of a peptide conjugate of formula (Ia) in which the inner core is loaded with SN-38 lactone wherein the concentration of the peptide conjugate of SN-38 in the micellar aqueous dispersion is 20 mg/ml and the concentration of SN-38 lactone is 4 mg/ml;
b) a micelle of a peptide conjugate of formula (Ia) in which the inner core is loaded with SN-38 lactone, wherein the concentration of the peptide conjugate of SN-38 in the micellar aqueous dispersion is 20 mg/ml and the concentration of SN-38 lactone is 2 mg/ml;
c) a micelle of a peptide conjugate of formula (Ia) in which the inner core is loaded with SN-38 lactone wherein the concentration of the peptide conjugate of SN-38 in the micellar aqueous dispersion is 10 mg/ml and the concentration of SN-38 lactone is 1 mg/ml;
d) a micelle of a peptide conjugate of formula (Ic) in which the inner core is loaded with SN-38 lactone wherein the concentration of the peptide conjugate of SN-38 in the micellar aqueous dispersion is 20 mg/ml and the concentration of SN-38 lactone is 2 mg/ml;
e) a micelle of a peptide conjugate of formula (Ia) in which the inner core is loaded with camptothecin lactone wherein the concentration of the peptide conjugate of SN-38 in the micellar aqueous dispersion is 20 mg/ml and the concentration of camptothecin lactone is 0.25-4 mg/ml; and
f) a micelle of a peptide conjugate of formula (Ia) in which the inner core is loaded with camptothecin lactone and SN-38, wherein the concentration of the peptide conjugate of SN-38 in the micellar aqueous dispersion is 20 mg/ml and the concentration of the camptothecin lactone is 0.5-1 mg/ml and the concentration of the SN-38 is 0.5-1 mg/ml.

12. The micelle according to claim 1, which is a freeze-dried micelle.

13. The micelle according to claim 1, which is obtainable by:
a) spontaneous self-assembly of a peptide conjugate of SN-38 as defined in claim 1 in water at pH<7;
b) contacting the micellar acidic solution with a basic solution containing free SN-38 carboxylate at concentrations of 1-12 mg/ml, which forms inter-molecular interactions between free SN-38 lactone molecules and the SN-38 molecule conjugated in the peptide conjugate of SN-38; and
c) optionally freeze-drying the micellar solution of step b).

14. A pharmaceutical composition comprising a therapeutically effective amount of micelles according to claim 1, together with appropriate amounts of pharmaceutically acceptable carriers or excipients.

15. A micelle according to claim 1 for use as a medicament.

16. A method for the treatment of cancer which comprises administering a micelle according to claim 1, to a mammal, wherein the cancer is selected from the group consisting of:
a) a tumor selected from the group consisting of extracranial solid tumors, eye tumors, and CNS tumors;
b) a cancer selected from the group consisting of adult glioma, pediatric gliomas, retinoblastoma, Ewing sarcoma, DIPG, neuroblastoma, medulloblastoma, ependymoma, atypical teratoid-rhabdoid tumors (ATRT) and rhabdomyosarcoma;
c) a pediatric brain tumor;
d) a pediatric high-grade glioma;
e) a Diffuse Intrinsic Pontine Glioma (DIPG) tumor; and
f) Diffuse Midline Glioma.

17. The micelle according to claim 2, wherein in the peptidic conjugate of SN-38 of formula (I), P is a biradical of a peptide selected from the group consisting of:
(a) a peptide which comprises the amino acid sequence DapKAPETALD with an intrapeptide bond between the Dap and D which is an amide bond, that is 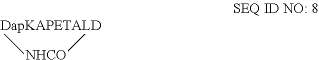
(b) a peptide having 9-20 amino acids residues in length having at least an intrapeptide bond, which is a disulfide bond, and comprises an amino acid sequence which is: CKAPETALCAAA having at least an intrapeptide disulfide bond between cysteines 1 and 9, that is 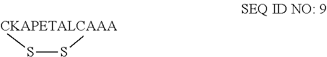
(c) a peptide having 9-11 amino acids residues in length having at least an intrapeptide bond which is a disulfide bond and consists of an amino acid sequence selected from the group consisting of CKAPETALC; CKAPETALCA; and CKAPETALCAA having at least an intrapeptide disulfide bond between cysteines 1 and 9, that are 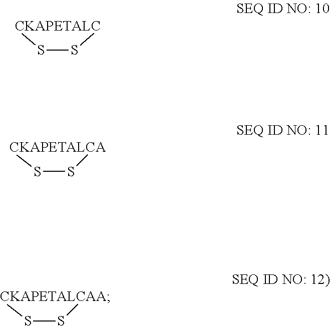
(d) a peptide which has 16 amino acid residues and comprises the amino acid sequence CNCKAPETALCAAACH with an intrapeptide disulfide bond between the first and third cysteine which are cysteines 1 and 11, and between the second and the fourth cysteine which are cysteine 3 and 15, that is, 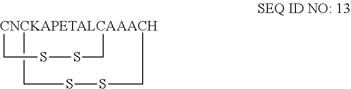
(e) a peptide which comprises the amino acid sequence DapKAPETALD (SEQ ID NO:14).

18. The micelle according to claim 8, which is in form of an aqueous dispersion of micelles as defined in claim 1, in which the free therapeutic anticancer drug is different.

19. The micelle according to claim 10, which is a freeze-dried micelle.

20. A pharmaceutical composition comprising a therapeutically effective amount of micelles according to claim 13, together with appropriate amounts of pharmaceutically acceptable carriers or excipients.


SEQUENCE LISTING (14 sequences)

SEQ ID NO: 1 - protein, 9 aa, synthetic construct
  REGION 1..9: note = PEPTIDE
  VARIANT 1: note = misc_feature - Xaa is selected from the group consisting of Dpr and Dbu, and it is bound to amino acid at position 9 through an amide intrapeptide bond
  VARIANT 9: note = misc_feature - Xaa is selected from the group consisting of Asp and Glu, and it is bound to amino acid at position 1 through an amide intrapeptide bond
XKAPETALX

SEQ ID NO: 2 - protein, 12 aa, synthetic construct
  REGION 1..12: note = PEPTIDE
  VARIANT 1: note = misc_feature - Xaa is an amino acid which is the same as the amino acid at position 9 and it is selected from cysteine, selenocysteine, and penicillamine
  VARIANT 1: note = misc_feature - it is bound to the amino acid at position 9 by a disulfide or diselenide intrapeptide bond
  VARIANT 9: note = misc_feature - Xaa is an amino acid which is the same as the amino acid at position 1 and it is selected from cysteine, selenocysteine, and penicillamine
  VARIANT 9: note = misc_feature - it is bound to the amino acid at position 1 by a disulfide or diselenide intrapeptide bond
XKAPETALXAAA

SEQ ID NO: 3 - protein, 9 aa, synthetic construct
  REGION 1..9: note = PEPTIDE
  VARIANT 1: note = misc_feature - Xaa is an amino acid which is the same as the amino acid at position 9 and it is selected from cysteine, selenocysteine, and penicillamine
  VARIANT 1: note = misc_feature - it is bound to the amino acid at position 9 by a disulfide or diselenide intrapeptide bond
  VARIANT 9: note = misc_feature - Xaa is an amino acid which is the same as the amino acid at position 1 and it is selected from cysteine, selenocysteine, and penicillamine
  VARIANT 9: note = misc_feature - it is bound to the amino acid at position 1 by a disulfide or diselenide intrapeptide bond
XKAPETALX

SEQ ID NO: 4 - protein, 10 aa, synthetic construct
  REGION 1..10: note = PEPTIDE
  VARIANT 1: note = misc_feature - Xaa is an amino acid which is the same as the amino acid at position 9 and it is selected from cysteine, selenocysteine, and penicillamine
  VARIANT 1: note = misc_feature - it is bound to the amino acid at position 9 by a disulfide or diselenide intrapeptide bond
  VARIANT 9: note = misc_feature - Xaa is an amino acid which is the same as the amino acid at position 1 and it is selected from cysteine, selenocysteine, and penicillamine
  VARIANT 9: note = misc_feature - it is bound to the amino acid at position 1 by a disulfide or diselenide intrapeptide bond
XKAPETALXA

SEQ ID NO: 5 - protein, 11 aa, synthetic construct
  REGION 1..11: note = PEPTIDE
  VARIANT 1: note = misc_feature - Xaa is an amino acid which is the same as the amino acid at position 9 and it is selected from cysteine, selenocysteine, and penicillamine
  VARIANT 1: note = misc_feature - it is bound to the amino acid at position 9 by a disulfide or diselenide intrapeptide bond
  VARIANT 9: note = misc_feature - Xaa is an amino acid which is the same as the amino acid at position 1 and it is selected from cysteine, selenocysteine, and penicillamine
  VARIANT 9: note = misc_feature - it is bound to the amino acid at position 1 by a disulfide or diselenide intrapeptide bond
XKAPETALXAA

SEQ ID NO: 6 - protein, 16 aa, synthetic construct
  REGION 1..16: note = PEPTIDE
  VARIANT 1: note = misc_feature - Xaa is an amino acid which is the same as the amino acid at position 11, and it is selected from the group consisting of cysteine, selenocysteine, and penicillamine
  VARIANT 1: note = misc_feature - it is bound to the amino acid at position 11 by a disulfide or diselenide intrapeptide bond
  VARIANT 3: note = misc_feature - Xaa is an amino acid which is the same as the amino acid at position 15, and it is selected from the group consisting of cysteine, selenocysteine, and penicillamine
  VARIANT 3: note = misc_feature - it is bound to the amino acid at position 15 by a disulfide or diselenide intrapeptide bond
  VARIANT 11: note = misc_feature - Xaa is an amino acid which is the same as the amino acid at position 1 and it is selected from the group consisting of cysteine, selenocysteine, and penicillamine
  VARIANT 11: note = misc_feature - it is bound to the amino acid at position 1 by a disulfide or diselenide intrapeptide bond
  VARIANT 15: note = misc_feature - Xaa is an amino acid which is the same as the amino acid at position 3, and it is selected from the group consisting of cysteine, selenocysteine, and penicillamine
  VARIANT 15: note = misc_feature - it is bound to the amino acid at position 3 by a disulfide or diselenide intrapeptide bond
XNXKAPETALXAAAXH

SEQ ID NO: 7 - protein, 9 aa, synthetic construct
  REGION 1..9: note = PEPTIDE
  VARIANT 1: note = MISC_FEATURE - Xaa is selected from Dpr and Dpu
  VARIANT 9: note = MISC_FEATURE - Xaa is selected from Glu and Asp
XKAPETALX

SEQ ID NO: 8 - protein, 9 aa, synthetic construct
  REGION 1..9: note = PEPTIDE
  VARIANT 1: note = misc_feature - Xaa represents Dap, and it is bound to the amino acid at position 9 by an amide intrapeptide bond
XKAPETALD

SEQ ID NO: 9 - protein, 12 aa, synthetic construct
  REGION 1..12: note = PEPTIDE
  DISULFID 1..9: note = intrapeptide disulfide bond between cysteins 1 and 9
CKAPETALCAAA

SEQ ID NO: 10 - protein, 9 aa, synthetic construct
  REGION 1..9: note = PEPTIDE
  DISULFID 1..9: note = intrapeptide disulfide bond between cysteins 1 and 9
CKAPETALC

SEQ ID NO: 11 - protein, 10 aa, synthetic construct
  REGION 1..10: note = PEPTIDE
  DISULFID 1..9: note = intrapeptide disulfide bond between cysteins 1 and 9
CKAPETALCA

SEQ ID NO: 12 - protein, 11 aa, synthetic construct
  REGION 1..11: note = PEPTIDE
  DISULFID 1..9: note = intrapeptide disulfide bond between cysteins 1 and 9
CKAPETALCAA

SEQ ID NO: 13 - protein, 16 aa, synthetic construct
  REGION 1..16: note = PEPTIDE
  DISULFID 1..11: note = intrapeptide disulfide bond between cysteins 1 and 11
  DISULFID 3..15: note = intrapeptide disulfide bond between cysteins 3 and 15
CNCKAPETALCAAACH

SEQ ID NO: 14 - protein, 9 aa, synthetic construct
  REGION 1..9: note = PEPTIDE
  VARIANT 1: note = misc_feature - Xaa means Dpr
XKAPETALD